(19) United States
(12) Patent Application Publication
(10) Pub. No.: US 2025/0331815 A1
(43) Pub. Date: Oct. 30, 2025

(54) WEARABLE APPARATUS FOR DEEP TISSUE SENSING AND DIGITAL AUTOMATION OF DRUG DELIVERY

(71) Applicant: The University of North Carolina at Chapel Hill, Chapel Hill, NC (US)

(72) Inventors: Wubin BAI, Chapel Hill, NC (US); Yihan LIU, Chapel Hill, NC (US); Yihang WANG, Chapel Hill, NC (US)

(21) Appl. No.: 18/866,958

(22) PCT Filed: May 18, 2023
(86) PCT No.: PCT/US2023/022771

§ 371 Date: Nov. 18, 2024

Related U.S. Application Data
(60) Provisional application No. 63/343,888, filed on May 19, 2022.

(51) Int. Cl. A61B 8/00; G16H 40/63

(52) U.S. Cl. CPC A61B 8/4236; A61B 8/4472; A61B 8/5269; A61B 8/54; G16H 40/63

(57) ABSTRACT

Various example of the present disclosure provide sensing apparatuses configured for wearable and wireless use for deep tissue physiological monitoring. The sensing apparatuses may be embodied by a thin flexible patch configured to conform with a skin surface of a subject. A sensing apparatus may include a plurality of microneedles oriented to extend towards and penetrate into the subject to a shallow depth. The microneedles may be configured as waveguides for a given sensing modality (e.g., light, ultrasound), such that sensing wave signals propagate to deep tissues. For the sensing, the sensing apparatus includes waveform generators (e.g., light-emitted diodes) and waveform detectors (e.g., photodiodes). Machine learning models may be used to process and denoise sampled data from the waveform detectors and to generate accurate and reliable physiological measurements, including heart rate, respiratory rate, pulse intensity, respiratory intensity, blood oximetry, tissue oximetry, blood flow rate, and/or the like.

### CROSS-REFERENCE TO RELATED APPLICATION

This application claims priority to and benefit of U.S. Provisional Patent Application No. 63/343,888, filed May 19, 2022, the entire content of which is incorporated by reference in its entirety.

### STATEMENT REGARDING FEDERALLY SPONSORED RESEARCH OR DEVELOPMENT

This invention was made with government support under Grant No. TR002489 awarded by National Institutes of Health. The government has certain rights in the invention.

### BACKGROUND

Continuous real-time monitoring of biological signals correlated with local regions and organs of a subject's body enhances both temporal and dimensional accuracy in many healthcare applications, including health monitoring and diagnosis of conditions (e.g., peripheral artery diseases). Relevant biological signals to be monitored are commonly present at depth within a subject at deep tissues such as musculature, circulatory vessels, and/or the like.

However, deep tissues having biological signals to be monitored are effectively shielded from the external environment of the subject by layers of skin and fatty issues, and these layers are generally attenuating, light-scattering, and wave-absorbing, thereby causing existing monitoring devices and systems to struggle with deep tissue sensing. For example, optical-based wearable devices positioned above and interfacing with a skin surface may lack sufficient ability to penetrate through cutaneous and subcutaneous layers to collect adequately interpretable data from deeper regions. Meanwhile, implantable devices may be inserted at depth through invasive surgical procedures to bypass such obstacles but are associated with a cost of significant and/or non-negligible infection and inflammation risk.

Thus, various technical challenges exist with deep tissue sensing and collection of biological signals at depth. Through applied effort, ingenuity, and innovation, the problems identified herein have been solved by developing solutions that are included in embodiments of the present disclosure, many examples of which are described in detail herein.

### BRIEF SUMMARY

Various embodiments of the present disclosure provide deep tissue sensing apparatuses, devices, methods of use thereof, computer program products, and/or the like that address technical challenges identified herein. Apparatuses and devices described herein are configured for wearable and wireless use to reliably and accurately collect biological signals and physiological measurement data from deep tissues. Various embodiments described herein incorporate biocompatible microneedles at a sensing interface, with the microneedles being configured as waveguides that enhance penetration of sensing wave signals. As a result, a sensing field may be expanded within subject tissue to thereby enable collection of deeper and more accurate physiological measurements for monitoring and detection applications. For example, various embodiments enable data collection with respect to tissue oximetry, pulse oximetry, heart pulsation, respiratory activities, photoplethysmography, and/or the like.

In particular, apparatuses described herein with various embodiments may include a multi-layer configuration in which a plurality of microneedles are attached to a base layer interfacing with a skin surface of a subject and oriented to extend into (e.g., penetration) the subject. The microneedles are configured with waveguiding properties such that sensing wave signals (e.g., light, ultrasonic waves) may propagate to depths further than the microneedles themselves. Through transmission of the sensing wave signals and detection of reflections thereof, generally, apparatuses can collect deep tissue data and are further configured to wireless communicate collected data with extremal systems and devices, in various embodiments. In various embodiments, sensing apparatuses are configured for safe wearable use with subjects; for example, the base layer of a sensing apparatus in accordance with various embodiments described herein may be configured to minimize a transfer of ambient heat from the sensing apparatus to the skin surface of the subject. Thus, as described within the present disclosure, various embodiments provide safe, wearable, and wireless solutions to deep tissue sensing that employ waveguiding microneedles to obtain reliable and accurate deep tissue biological data.

The following are some example embodiments in accordance with the present disclosure. It is noted that the scope of the present disclosure is not limited to these example embodiments.

Embodiment 1: a sensing apparatus comprises: a base layer configured to interface with a skin surface of a subject; a sensing layer positioned above the base layer and comprising one or more waveform detectors and one or more waveform generators configured to emit wave signals; and a plurality of microneedles attached to a skin-interfacing portion of the base layer and oriented to extend into at least a dermal depth and/or a subcutaneous depth of the subject, wherein the plurality of microneedles are configured to waveguide the wave signals into a deep tissue of the subject.

Embodiment 2: the sensing apparatus of any of the preceding embodiments, wherein the one or more waveform generators comprise one or more light-emitting diodes configured to emit light signals, and wherein the plurality of microneedles are configured as optical waveguides for the light signals.

Embodiment 3: the sensing apparatus of any of the preceding embodiments, wherein the light signals include visible red light signals and near-infrared signals.

Embodiment 4: the sensing apparatus of any of the preceding embodiments, wherein the one or more waveform generators comprise one or more ultrasonic generators configured to emit ultrasonic signals, and wherein the plurality of microneedles are configured to act as ultrasonic waveguides for the ultrasonic signals.

Embodiment 5: the sensing apparatus of any of the preceding embodiments, further comprising: a control module in electronic communication with the one or more waveform generators and the one or more waveform detectors, the control module configured to: operate the one or more waveform generators to define a sensing field within the deep tissue of the subject via the wave signals and the plurality of microneedles as waveguides; and generate sensing data based at least in part on reflected wave signals detected at the one or more waveform detectors.

Embodiment 6: the sensing apparatus of any of the preceding embodiments, wherein the control module is positioned above the sensing layer.

Embodiment 7: the sensing apparatus of any of the preceding embodiments, wherein the control module is further configured to process the sensing data to determine physiological measurements associated with the deep tissue of the subject, the physiological measurements selected from the group consisting of at least one of tissue oximetry measurements, pulse oximetry measures, heart pulsation measurements, respiratory measurements, volume measurements, or plethysmographic measurements.

Embodiment 8: the sensing apparatus of any of the preceding embodiments, wherein the physiological measurements are determined from the sensing data using one or more machine learning models trained at least to reduce noise in the sensing data.

Embodiment 9: the sensing apparatus of any of the preceding embodiments, wherein the control module is further configured to transmit, via wireless communication, the sensing data and/or the physiological measurements to a workstation.

Embodiment 10: the sensing apparatus of any of the preceding embodiments, wherein the base layer and the plurality of microneedles are configured to minimize a transfer of ambient heat originating from the one or more waveform generators to the skin surface of the subject.

Embodiment 11: the sensing apparatus of any of the preceding embodiments, wherein at least the base layer and the sensing layer form a flexible substrate configured to conform to contours of the skin surface of the subject.

Embodiment 12: the sensing apparatus of any of the preceding embodiments, wherein the plurality of microneedles are comprised of biocompatible material with waveguiding properties.

Embodiment 13: a system for deep tissue sensing for a subject, the system comprising: a sensing apparatus secured to the subject, the sensing apparatus comprising: a sensing layer comprising one or more waveform detectors and one or more waveform generators configured to emit wave signals, a plurality of microneedles configured to extend into at least a dermal depth and/or a subcutaneous depth and configured to waveguide the wave signals into a deep tissue of the subject, and a control unit configured to generate and transmit, via wireless communication, sensing data based at least in part on reflected wave signals detected at the one or more waveform detectors; and a workstation configured to: receive, via wireless communication, the sensing data from the sensing apparatus, and determine a plurality of physiological measurements associated with the deep tissue of the subject from the sensing data.

Embodiment 14: the system of any of the preceding embodiments, wherein the physiological measurements are determined using one or more machine learning models trained at least to reduce noise in the sensing data.

Embodiment 15: the system of any of the preceding embodiments, wherein the physiological measurements are selected from the group consisting of at least one of tissue oximetry measurements, pulse oximetry measures, heart pulsation measurements, respiratory measurements, volume measurements, or plethysmographic measurements.

Embodiment 16: the system of any of the preceding embodiments, wherein the workstation is configured to receive the sensing data over time for continuous monitoring of the subject.

Embodiment 17: an apparatus comprising at least one processor and at least one memory having computer program code stored thereon, the at least one memory and the computer program code configured to, with the at least one processor, cause the apparatus to: determine a sensing field for a deep tissue of a subject; cause one or more waveform generators to emit wave signals that propagate through the deep tissue of the subject to define the sensing field based at least in part on being waveguided by a plurality of microneedles; and generate sensing data from reflected wave signals detected at one or more waveform detectors and originating from the sensing field.

Embodiment 18: the apparatus of any of the preceding embodiments, wherein the at least one memory and the computer program code are further configured to, with the at least one processor, cause the apparatus to: transmit, via wireless communication, the sensing data to a workstation configured to determine a plurality of physiological measurements associated with the deep tissue of the subject from the sensing data.

Embodiment 19: the apparatus of any of the preceding embodiments, wherein the apparatus is secured to the subject with the one or more waveform generators, the plurality of microneedles, and the one or more waveform detectors.

Embodiment 20: the apparatus of any of the preceding embodiments, wherein the plurality of microneedles are configured to extend past a skin surface of the subject to at least a dermal depth.

Embodiment 21: an apparatus for transdermal delivery comprising: a microneedle; and an electrically triggerable membrane encapsulating the microneedle and defining at least one reservoir between the microneedle and the electrically triggerable membrane.

Embodiment 22: the apparatus of any of the preceding embodiments, wherein the electrically triggerable membrane comprises electrically triggerable gold.

Embodiment 23: the apparatus of any of the preceding embodiments, wherein a membrane width associated with the electrically triggerable membrane is between 145 nanometers and 155 nanometers.

Embodiment 24: the apparatus of any of the preceding embodiments, further comprising: a controller coupled to the microneedle and configured to transmit an electrical trigger to the microneedle to cause a disintegration of the electrically triggerable membrane and a release of content from the at least one reservoir.

Embodiment 25: the apparatus of any of the preceding embodiments, wherein the electrical trigger comprises a direct current signal between 2 volts and 3 volts.

Embodiment 26: the apparatus of any of the preceding embodiments, wherein the controller is configured to: receive a release control signal, and in response to the release control signal, transmit the electrical trigger to the microneedle.

Embodiment 27: the apparatus of any of the preceding embodiments, the controller comprises at least one of a near-field communication module or a Bluetooth module.

Embodiment 28: the apparatus of any of the preceding embodiments, wherein the release control signal comprises a microneedle indication associated with the microneedle.

Embodiment 29: the apparatus of any of the preceding embodiments, further comprising: a microneedle array comprising a plurality of microneedles that includes the microneedle, wherein the electrically triggerable membrane encapsulates each of the plurality of microneedles.

Embodiment 30: the apparatus of any of the preceding embodiments, wherein the controller is configured to: receive a plurality of release control signals; determine one or more microneedles from the plurality of microneedles that are associated with the plurality of release control signals; and transmit one or more electrical triggers to the one or more microneedles.

Embodiment 31: a sensing apparatus for deep tissue sensing and transdermal delivery, the sensing apparatus comprising: a base layer configured to interface with a skin surface of a subject; a sensing layer positioned above the base layer and comprising one or more waveform detectors and one or more waveform generators configured to emit wave signals; a microneedle attached to a skin-interfacing portion of the base layer and configured to waveguide the wave signals into a deep tissue of the subject; and an electrically triggerable membrane encapsulating the microneedle and defining at least one reservoir between the microneedle and the electrically triggerable membrane.

Embodiment 32: the sensing apparatus of any of the preceding embodiments, further comprising: a controller coupled to the microneedle and configured to transmit an electrical trigger to the microneedle to cause a disintegration of the electrically triggerable membrane and a release of content from the at least one reservoir.

Embodiment 33: the sensing apparatus of any of the preceding embodiments, wherein the controller is configured to: receive a release control signal, and in response to the release control signal, transmit the electrical trigger to the microneedle.

Embodiment 34: the sensing apparatus of any of the preceding embodiments, wherein the release control signal comprises a microneedle indication associated with the microneedle.

Embodiment 35: the sensing apparatus of any of the preceding embodiments, further comprising: a microneedle array comprising a plurality of microneedles that includes the microneedle, wherein the electrically triggerable membrane encapsulates each of the plurality of microneedles.

Embodiment 36: the sensing apparatus of any of the preceding embodiments, wherein the one or more waveform generators comprise one or more light-emitting diodes configured to emit light signals, and wherein the microneedle is configured as an optical waveguide for the light signals.

Embodiment 37: the sensing apparatus of any of the preceding embodiments, wherein the light signals include visible red light signals and near-infrared signals.

Embodiment 38: the sensing apparatus of any of the preceding embodiments, wherein the one or more waveform generators comprise one or more ultrasonic generators configured to emit ultrasonic signals, and wherein the microneedle is configured to act as ultrasonic waveguides for the ultrasonic signals.

Embodiment 39: the sensing apparatus of any of the preceding embodiments, further comprising: a controller in electronic communication with the one or more waveform generators and the one or more waveform detectors, the controller is configured to: operate the one or more waveform generators to define a sensing field within the deep tissue of the subject via the wave signals and the microneedle as a waveguide; and generate sensing data based at least in part on reflected wave signals detected at the one or more waveform detectors.

Embodiment 40: the sensing apparatus of any of the preceding embodiments, wherein the controller is further configured to transmit, via wireless communication, the sensing data to a workstation.

### BRIEF DESCRIPTION OF THE DRAWINGS

Having thus described the present disclosure in general terms, reference will now be made to the accompanying drawings, which are not necessarily drawn to scale.

FIG. 1A and FIG. 1B are diagrams of system architectures that can be used in conjunction with various embodiments of the present disclosure;

FIG. 2A provides a top view of an example sensing apparatus configured for wearable and wireless use for deep tissue sensing applications, in accordance with various embodiments described herein;

FIG. 2B illustrates wearable use and tissue interfacing of an example sensing apparatus with a subject for deep tissue sensing, in accordance with various embodiments described herein;

FIG. 2C illustrates mechanical flexibility featured in an example sensing apparatus configured for wearable and wireless use for deep tissue sensing applications, in accordance with various embodiments described herein;

FIG. 2D includes an exploded view of various components of an example sensing apparatus configured for wearable and wireless use for deep tissue sensing applications, in accordance with various embodiments described herein;

FIG. 2E demonstrates an example application of sensing wave signals for collected oximetry-related measurements, in accordance with various embodiments described herein;

FIG. 2F provides a perspective view of an example array of microneedles configured to waveguide sensing wave signals to deep tissue depths, in accordance with various embodiments described herein;

FIG. 2G provides an example scanning electron microscope (SEM) image of at least a portion of an example array of microneedles configured to provide sensing wave signals to deep tissue depths, in accordance with various embodiments described herein;

FIG. 2H provides an exploded view of an example wearable use and tissue interfacing of components of an example sensing apparatus configured for wearable and wireless use for deep tissue sensing applications, in accordance with various embodiments described herein;

FIG. 3 is a schematic of a computing entity that may be used in conjunction with various embodiments of the present disclosure;

FIG. 4A provides a block diagram describing various example operations performed by components of an example sensing apparatus and/or one or more external devices in accordance with various embodiments of the present disclosure;

FIG. 4B provides a block diagram describing various example operations performed by an example sensing apparatus to generate deep tissue physiological measurements in accordance with various embodiments of the present disclosure;

FIG. 4C illustrates example filter data used in various example operations performed by an example sensing apparatus to generate deep tissue physiological measurements in accordance with various embodiments of the present disclosure;

FIG. 5A provides a diagram illustrating an example architecture for an autoencoder-based machine learning model used to process collected data in conjunction with various embodiments of the present disclosure;

FIG. 5B and FIG. 5C include example data demonstrating denoising application of an example autoencoder-based machine learning model in accordance with various embodiments of the present disclosure;

FIG. 6A illustrates an example process for fabricating an array of waveguiding microneedles in accordance with various embodiments of the present disclosure;

FIG. 6B describes transmittance values for materials that constitute waveguiding microneedles in accordance with various embodiments of the present disclosure;

FIG. 7A, FIG. 7B, FIG. 7C, FIG. 7D, and FIG. 7E illustrate improvements to a sensing field resulting from the implementation of waveguiding microneedles in accordance with various embodiments of the present disclosure;

FIG. 8A provides a diagram illustrating an example layout of microneedles, waveform generators, and waveform detectors across a sensing apparatus in accordance with various embodiments of the present disclosure;

FIG. 8B includes cross-sectional views of a sensing field resulting from the example layout of microneedles, waveform generators, and waveform detectors described by FIG. 8A;

FIG. 9A illustrates a compression test performed on an array of microneedles to validate skin penetration capability in accordance with various embodiments of the present disclosure;

FIG. 9B provides experimental data related to compression and bending of an example array of microneedles in accordance with various embodiments of the present disclosure;

FIG. 9C illustrates a bending test performed on an array of microneedles in accordance with various embodiments of the present disclosure;

FIG. 10A and FIG. 10B demonstrate temperature insulation provided at the skin interface of a sensing apparatus due at least in part on implementation of microneedles in accordance with various embodiments of the present disclosure;

FIG. 11A and FIG. 11B include example photovoltage data sampled based at least in part on different configurations and layouts of waveform generators and waveform detectors in accordance with various embodiments of the present disclosure;

FIG. 12A illustrates in vivo testing of different wearable uses of a sensing apparatus in accordance with various embodiments of the present disclosure;

FIG. 12B provides generated physiological measurements obtained via different wearable uses of a sensing apparatus in accordance with various embodiments of the present disclosure;

FIG. 12C provides a diagram illustrating multi-contact interfacing of the sensing apparatus to enable simultaneous generation of physiological measurements in accordance with various embodiments of the present disclosure;

FIG. 12D illustrates different wearable uses of a sensing apparatus in accordance with various embodiments of the present disclosure;

FIG. 13A illustrates in vivo testing of a sensing apparatus with a rat model for diagnosis of a peripheral arterial disease in accordance with various embodiments of the present disclosure;

FIG. 13B illustrates time-course data for various physiological measurements obtained via the sensing apparatus in accordance with various embodiments of the present disclosure;

FIG. 13C provides example collected photovoltage data at different subject conditions in accordance with various embodiments of the present disclosure;

FIG. 14 describes power source lifetime of a sensing apparatus in accordance with various embodiments of the present disclosure.

FIG. 15A provides an example schematic illustration highlighting an example construction of an example wirelessly controlled spatiotemporal on-demand patch (SOP) for high-precision drug delivery in accordance with various embodiments of the present disclosure.

FIG. 15B provides an example exploded view of an example drug-delivery interface of the SOP, including a polydimethylsiloxane (PDMS) encapsulation, an example electrically triggerable gold (Au) coating, drug-loaded microneedles based on poly(D,L-lactide-co-glycolide) (PLGA), and a PLGA substrate in accordance with various embodiments of the present disclosure.

FIG. 15C provides an example schematic illustration showing an example process of electrically controlled on-demand drug delivery from an individual microneedle in accordance with various embodiments of the present disclosure.

FIG. 15D provides an example schematic illustration demonstrating an example spatiotemporal control of releasing profile from an example SOP in accordance with various embodiments of the present disclosure.

FIG. 15E provides an example schematic illustration demonstrating an example spatiotemporal control of releasing profile from an example SOP in accordance with various embodiments of the present disclosure.

FIG. 15F provides an example schematic illustration demonstrating an example spatiotemporal control of releasing profile from an example SOP in accordance with various embodiments of the present disclosure.

FIG. 15G provides an example schematic illustration demonstrating an example spatiotemporal control of releasing profile from an example SOP in accordance with various embodiments of the present disclosure.

FIG. 15H provides an example optical image of an example PLGA microneedle array in accordance with various embodiments of the present disclosure.

FIG. 15I provides an example corresponding SEM image with an example tilted view on the example PLGA microneedle array in accordance with various embodiments of the present disclosure.

FIG. 15J provides an optical image of an example PLGA microneedle array loaded with Rhodamine B in accordance with various embodiments of the present disclosure.

FIG. 15K provides an optical image of an example PLGA microneedle array protected with an electrically triggerable encapsulation in accordance with various embodiments of the present disclosure.

FIG. 16A provides example optical images and the corresponding example SEM image to demonstrate an example SOP undergoing an example process of electrically controlled crevice corrosion in accordance with various embodiments of the present disclosure.

FIG. 16B provides an example tip area analysis of microneedle in accordance with various embodiments of the present disclosure.

FIG. 16C provides an example waist area analysis of microneedle in accordance with various embodiments of the present disclosure.

FIG. 16D provides an example schematic illustration indicating the corresponding areas of an example microneedle analyzed by energy-dispersive X-ray spectroscopy (EDXS) element mapping in accordance with various embodiments of the present disclosure.

FIG. 16E provides an example amperometry characterization of the crevice corrosion process of the gold layer (150 nm) coated on microneedles with 1.2 mm height in accordance with various embodiments of the present disclosure.

FIG. 16F provides an example measured relationship between corrosion time and trigger potential applied on the gold layer in accordance with various embodiments of the present disclosure.

FIG. 16G provides an example analysis of thermal effect during an example crevice corrosion process, indicating negligible heat generated in the system, in accordance with various embodiments of the present disclosure.

FIG. 16H provides an example simulation of crevice corrosion depth on microneedle under 2.5-V in 1× Dulbecco's phosphate-buffered saline (DPBS) in accordance with various embodiments of the present disclosure.

FIG. 16I provides an example corresponding corrosion profile on a microneedle in 1 min (2.5-V, 1× Dulbecco's phosphate-buffered saline) in accordance with various embodiments of the present disclosure.

FIG. 17A provides example optical images of microneedles with gold (“Au”) coating and without Au coating in accordance with various embodiments of the present disclosure.

FIG. 17B provides an encapsulation profile of 100 nm Au layer on 1.2-mm microneedle in 40° C. 1× Dulbecco's phosphate-buffered saline in accordance with various embodiments of the present disclosure.

FIG. 17C provides an example optical image of an example wireless SOP in accordance with various embodiments of the present disclosure.

FIG. 17D provides an example power stability measurement of an example wireless SOP in accordance with various embodiments of the present disclosure.

FIG. 17E provides an example component illustration of an example wirelessly powered SOP in accordance with various embodiments of the present disclosure.

FIG. 17F provides an example frequency matching characterization of an example wireless power transfer in accordance with various embodiments of the present disclosure.

FIG. 17G provides an example corresponding circuit diagram of an example SOP in accordance with various embodiments of the present disclosure.

FIG. 18A provides an example schematic illustration of an example multi-domain SOP in accordance with various embodiments of the present disclosure.

FIG. 18B provides an example stepwise release profile of an example multistage drug release simulated by Rhodamine B in accordance with various embodiments of the present disclosure.

FIG. 18C provides an example schematic illustration showing an example sequential electrical-triggering schedule on an example multi-array SOP in accordance with various embodiments of the present disclosure.

FIG. 18D provides example optical images of an example multi-domain SOP undergoing an example sequential electrical trigger in accordance with various embodiments of the present disclosure.

FIG. 19A provides example optical images of an example intracranial microneedle in accordance with various embodiments of the present disclosure.

FIG. 19B provides an example measured force-displacement curve of an example microneedle array (for example, 9-needle, 1.2-mm in length) during an example fracture test in accordance with various embodiments of the present disclosure.

FIG. 19C provides an example schematic illustration of an example brain model indicating deployment location of example SOP in an in vivo study in accordance with various embodiments of the present disclosure.

FIG. 19D provides example measured pulsatile triggers generated from an example SOP microneedle at various distances to the example microneedle in accordance with various embodiments of the present disclosure.

FIG. 19E provides measured example square wave triggers generated from an example SOP microneedle at various distances to an example microneedle in accordance with various embodiments of the present disclosure.

FIG. 19F provides example optical images of an example microneedle (3-mm) before the test, after pulses triggers, and after square wave triggers in accordance with various embodiments of the present disclosure.

FIG. 19G illustrates mouse after example SOP deployment in accordance with various embodiments of the present disclosure.

FIG. 19H provides example immunohistochemical staining images of an example recovery process after example microneedle implantation in accordance with various embodiments of the present disclosure.

FIG. 20 provides an example schematic illustration of an example fabrication process in accordance with various embodiments of the present disclosure.

FIG. 21A provides example optical images of PLGA microneedles in accordance with various embodiments of the present disclosure.

FIG. 21B provides example SEM images of PLGA microneedles from the 45-degree perspective in accordance with various embodiments of the present disclosure.

FIG. 21C provides example SEM images of PLGA microneedles from the top perspective in accordance with various embodiments of the present disclosure.

FIG. 21D provides example SEM images of PLGA microneedles from the horizontal perspective in accordance with various embodiments of the present disclosure.

FIG. 21E provides example optical images of gold coated microneedles in accordance with various embodiments of the present disclosure.

FIG. 21F provides example images illustrating example measurements of an example base diameter of microneedles in accordance with various embodiments of the present disclosure.

FIG. 21G provides example images illustrating example measurements of an example length of microneedles in accordance with various embodiments of the present disclosure.

FIG. 21H provides an example statistical analysis of example base diameters of different microneedles in accordance with various embodiments of the present disclosure.

FIG. 21I provides an example statistical analysis of example length of different microneedles in accordance with various embodiments of the present disclosure.

FIG. 21J provides an example laser pattern for UV ablation of an example microneedle with an example base diameter of 270 μm in accordance with various embodiments of the present disclosure.

FIG. 22A provides example optical images of a 5 by 5 1.5-mm microneedle array before and after an example fracture test in accordance with various embodiments of the present disclosure.

FIG. 22B provides an example mechanical testing curve for the example 25-microneedle array in accordance with various embodiments of the present disclosure.

FIG. 22C provides example optical images of a 3 by 3 1.2-mm microneedle array before and after example fracture test in accordance with various embodiments of the present disclosure.

FIG. 22D provides an example mechanical testing curve for the example 9-microneedle array in accordance with various embodiments of the present disclosure

FIG. 22E provides example optical images of an example 5 by 5 1.5-mm microneedle array before and after an example agarose penetration test in accordance with various embodiments of the present disclosure.

FIG. 22F provides an example mechanical testing curve for the example 25-microneedle array on 0.5% agarose in accordance with various embodiments of the present disclosure.

FIG. 22G provides example optical images of example stages of agarose penetration test in accordance with various embodiments of the present disclosure.

FIG. 23A provides example optical images of example microneedles from the standby stage to the releasing stage in accordance with various embodiments of the present disclosure.

FIG. 23B provides example SEM images of microneedles from the standby stage to the releasing stage based on the horizontal perspective in accordance with various embodiments of the present disclosure.

FIG. 23C provides example SEM images of microneedles from the standby stage to the releasing stage based on the 45-degree perspective in accordance with various embodiments of the present disclosure.

FIG. 23D provides example SEM images of microneedles from the standby stage to the releasing stage based on the top perspective in accordance with various embodiments of the present disclosure.

FIG. 24 provides an example EDXS spectra of example microneedles from different stages of electrochemical corrosion on two areas (the top area and the waist area) in accordance with various embodiments of the present disclosure

FIG. 25A provides example high magnification SEM images, oxygen, and carbon mapping of an example tip area of microneedles from three stages in accordance with various embodiments of the present disclosure.

FIG. 25B provides example high magnification SEM image, oxygen, and carbon mapping of an example waist area of microneedles from three stages in accordance with various embodiments of the present disclosure.

FIG. 26A provides an example roughness analysis of gold surface during corrosion in accordance with various embodiments of the present disclosure.

FIG. 26B provides an example schematic illustration of the experiment setup in accordance with various embodiments of the present disclosure.

FIG. 26C provides an example zoom-in view of the experimental device in accordance with various embodiments of the present disclosure.

FIG. 26D provides example optical images of an example exposed gold region on anode during crevice corrosion in accordance with various embodiments of the present disclosure.

FIG. 27A provides an example optical image of a 3 by 3 100-nm Mo coated microneedle array (1.2-mm) in accordance with various embodiments of the present disclosure.

FIG. 27B provides an example amperometry characterization of the electrochemical crevice corrosion of Mo layer on microneedles under different potentials in 1×DPBS in accordance with various embodiments of the present disclosure.

FIG. 27C illustrates an example relationship between corrosion time and potential in accordance with various embodiments of the present disclosure.

FIG. 28A provides example optical images of an example microneedle array undergoing dye release from 0 to 60 minutes in 45° C. 1×DPBS in accordance with various embodiments of the present disclosure.

FIG. 28B provides an example UV-Vis spectroscopy (300-800 nm) of Rhodamine B standard solutions (Sample 1-6) in accordance with various embodiments of the present disclosure.

FIG. 28C illustrates an example calibration curve of Rhodamine B in accordance with various embodiments of the present disclosure.

FIG. 28D provides example data points associated with UV-Vis spectroscopy of the environment solution in accordance with various embodiments of the present disclosure.

FIG. 28E illustrates example relationships between absorbance and time of environment solution from the dye release experiment in accordance with various embodiments of the present disclosure.

FIG. 29A provides an example schematic illustration of example electrical triggers at the single-needle level in accordance with various embodiments of the present disclosure.

FIG. 29B illustrates example optical images of an example 8-needle device during multistage triggering on single microneedles of an example patch in accordance with various embodiments of the present disclosure.

FIG. 29C provides example optical images of an example 7-microneedle array before and after electrical triggering in accordance with various embodiments of the present disclosure.

FIG. 30A provides an example power transfer efficiency graph in accordance with various embodiments of the present disclosure.

FIG. 30B illustrates example relationships between output voltage and frequency in accordance with various embodiments of the present disclosure.

FIG. 30C illustrates an example Bode plot of an example SOP system in accordance with various embodiments of the present disclosure.

FIG. 30D provides an example Nyquist plot of an example SOP system in accordance with various embodiments of the present disclosure.

FIG. 31 provides example representative confocal images of example 40-μm horizontal cortical slices at various stages after implantation of example bioresorbable electrode probes in accordance with various embodiments of the present disclosure.

FIG. 32 provides examples images associated with an example SOP coupled with an example custom head stage that can be firmly mounted onto mice heads in accordance with various embodiments of the present disclosure.

FIG. 33A, FIG. 33B, FIG. 33C, FIG. 33D, FIG. 33E, FIG. 33F, FIG. 33G, FIG. 33H, FIG. 33I, FIG. 33J, FIG. 33K, and FIG. 33L provide example diagrams showing example in vitro stimulations of brain with microneedles in accordance with various embodiments of the present disclosure.

FIG. 34A, FIG. 34B, FIG. 34C, and FIG. 34D provide example finite element analyses of an example two-microneedle model in accordance with various embodiments of the present disclosure.

FIG. 34E provides an example anodic polarization curve with standardized current of an example crevice corrosion in accordance with various embodiments of the present disclosure.

FIG. 34F and FIG. 34G illustrates an example diagram showing an example corrosion rate of gold at 2.5 V and an example corresponding visualization in accordance with various embodiments of the present disclosure.

### DETAILED DESCRIPTION

Various embodiments of the present disclosure now will be described more fully hereinafter with reference to the accompanying drawings, in which some, but not all embodiments of the disclosure are shown. Indeed, the disclosure may be embodied in many different forms and should not be construed as limited to the embodiments set forth herein; rather, these embodiments are provided so that this disclosure will satisfy applicable legal requirements. The term “or” (also designated as “/”) is used herein in both the alternative and conjunctive sense, unless otherwise indicated. The terms “illustrative” and “exemplary” are used to be examples with no indication of quality level. Like numbers refer to like elements throughout.

### I. General Overview and Technical Improvements

Technologies that can better inform early diagnosis and proactive treatments for acute syndromes of vital diseases represent an essential keystone to create temporally resolved therapeutics that can further reduce experienced pains, prevent fatal events, and improve the wellbeing of individual life. Peripheral artery disease (PAD) represents just one significant example with unmet needs for such technology. PAD is a family of disorders that cause stenosis or thrombus in the arteries/aorta of the limbs, compromises the physiological functions of the human extremities, and leads to symptoms including atypical leg pain and claudication. PAD harms an individual not only through its direct symptoms, but also by increasing the likelihood of myocardial infarction, ischemic stroke, and other cardiovascular diseases, which could further evolve into a prevalent factor of mobility impairment, mental issues, and mortality, especially in the elderly. As with those of other conditions and diseases, early warning signs correlated with PAD are often neglected or underappreciated, especially in low-resource settings, despite being valuable in informing timely actions of preventive therapeutics before complications escalate.

Detection of some example warning signs and indicators for various conditions such as PAD is associated with several major technical limitations, including long turnaround time to yield one result. For example, ankle-brachial index (ABI), or the ratio of blood pressure measured at the ankle to the upper arm, takes approximately 15 to 30 minutes for a reading, thereby making it only applicable for weekly or even monthly reexamination for PAD subjects with mild or no symptoms and challenging for critical PAD subjects who have trouble engaging in daily activities or live in high health risk. Further technical limitations exist in inaccurate measurements with interference from vessel pulsation, making symptom identification difficult for PAD subjects with only mild stenosis or non-compressible vessels (due to other illnesses like diabetes). Another technical challenge presents in motion sensitivity, forcing detection to be strongly reliant on a fully steady status to avoid false tests and diagnostic misjudgment.

Existing developments in soft electronics with built-in systems on chip (SoC) enable opportunities for continuous, real-time monitoring of physiological conditions of targeted tissue regimes without excessive disturbance to user comfort and daily activities. The use of wearable biosensors allows subjects (e.g., patients) to be managed at home, thereby reducing burdens and barriers concerning access to healthcare, subject adherence, and cost. Examples such as smartwatches incorporate movement and exercise sensing functions and detect vital signs including respiratory rate, skin temperature, global pulse oxygenation, heart rhythms, and/or the like.

However, this skin-interfaced modality couples with technical challenges for the wearable devices to sense physiological parameters (e.g., oxygenation) of tissue microenvironments at depth. Implantable biosensors, such as injectable near-infrared probes, millimeter-scale ultrasonic chips, implantable micro-electromechanical systems, and/or the like, provide solutions for continuous, deep tissue monitoring, but the invasive procedures required for deployment and extraction of such implantable biosensors are associated with potential damages and infections to neighboring tissue. The risks associated with the invasiveness of implantable biosensors preclude their implementation for a broader range of diseases, other than those that require major surgeries already.

Moreover, implantable electronics place a strong requirement on the constituent materials to be highly biocompatible, which further limits choices of electronic materials. Considering that many example device capabilities typically rely on an integrated chip (e.g., for on-chip data) or similar electronic designs, existing solutions either sacrifice the capability of on-chip data processing by being fully tethered and reliant on an external and off-device system for data analysis or by processing collected data in situ by a partially wired design in which the data is processed and advertised on a portable on-skin circuit. However, both these and similar strategies may generate additional complications and risks to the body and strongly interfere with user comfort.

To address at least these technical challenges, various embodiments of the present disclosure provide sensing apparatuses and devices for wearable and wireless use in deep tissue sensing, as well as methods, operations, computer program products, and systems for the same. In particular, various example embodiments provide a wearable wireless patch that incorporates microneedles configured with waveguiding properties to enable deep propagation of sensing wave signals to deep tissue depths. Accordingly, deep tissue physiological parameters such as oxygenation may be detected without implantation procedures, while also overcoming attenuation-related challenges associated with cutaneous and subcutaneous tissue layers. Various embodiments described herein may be configured for multi-modality measurements from different sensing points to yield both local and global physiological information continuously and simultaneously. According to example embodiments, the sensing apparatus includes arrays of one or more light-emitting diodes (LEDs) and arrays of one or more photodiodes for light-based sensing that is also location-dependent. In further example embodiments, sensing apparatuses may generally include waveform generators and waveform detectors for other modalities such as ultrasound or ultrasonic-based sensing in alternative to or in combination with the light-based sensing. For example, the one or more waveform generators comprise one or more ultrasonic generators configured to emit ultrasonic signals. The sensing interface relies on biocompatible waveguides in the form of microneedles that are composed of FDA-approved biocompatible material, which is a material that may not result in significant inflammation or infection within a subject (e.g., a human). Examples of biocompatible material may include, but are not limited to, poly(lactic-co-glycolic acid) (PLGA). In some embodiments, the microneedles extend into the subject at a shallow depth while waveguiding and propagating sensing wave signals to deeper depths. For example, the microneedles of a sensing apparatus are optical waveguides for light signals and may be transparent. Additionally, or alternatively, the microneedles of a sensing apparatus are ultrasonic waveguides for the ultrasonic signals.

Generally, the microneedles provide paths, fields, or means of minimal attenuation to ensure efficient delivery of sensing wave signals directly into deep tissue.

Further addressing the identified technical challenges, various embodiments include on-device computing and control. In various embodiments, the sensing apparatus is equipped with a control module that provides a series of signal pre-processing, signal processing, and/or wireless communication interfaces and in electronic communication with the one or more waveform generators and the one or more waveform detectors. In some embodiments, the control module may operate the one or more waveform generators to define a sensing field within the deep tissue of the subject via the wave signals and the plurality of microneedles as waveguides. For example, a sensing field for a deep tissue of a subject can be determined based on the propagation field of the wave signals generated by the one or more waveform generators and guided through the plurality of microneedles. In some embodiments, the control module may generate sensing data based at least in part on reflected wave signals detected at the one or more waveform detectors and originating from the sensing field. In some embodiments, the control module is positioned above the sensing layer of the sensing apparatus. In some examples, the sensing apparatus may be configured to advertise collected data for further processing by an external computing entity. To support operation of at least the control module, waveform generators (e.g., LEDs), and waveform detectors (e.g., photodiodes), the sensing apparatus may include a portable rechargeable battery. Overall, the sensing apparatus may be packaged and fully encapsulated for safety and longevity.

Furthermore, to minimize the noise raised during the data capturing process with low delay, machine learning models may be applied to reconstruct collected data, such as photovoltage data in light-based sensing applications. For example, an autoencoder model and/or a neural network model configured for denoising may be recruited, trained, and/or used. Depending on the embodiment, such machine learning models may specifically be used at the control module of the sensing apparatus and/or one or more external computing entities. Machine learning models provide technical benefits in the context of denoising collected data (e.g., photovoltage data), as user adjustment of filtering criteria may be obviated in certain measurement conditions, in contrast to conventional filters (e.g., the Butterworth filter, the Savitzky-Golay filter). Furthermore, with the right training, machine learning models can potentially handle data smoothing in a broader range of measurement circumstances, making it a more generalizable tool for bio-signal processing with the sensing apparatus, in various embodiments.

Simulated results described herein demonstrate and quantify the amplification effect brought by the array(s) of waveguiding microneedles, and experimental results also described herein from execution on artificial skin tissue provides further confirmation. In light-based sensing applications, for example, the irradiance distributed on the vertical profile at LED locations validates that the penetration depth (e.g., defined by a 1/10 loss due to the light extinction in skin tissue) of red and near-infrared light are enhanced by 6.3 and 4.0 times, respectively. Meanwhile, experimental results from mechanical and thermal tests described herein corroborate that the physical performances of the microneedles match the required clinical relevance as a transmission medium between the oximeter and skin. In vivo demonstration using a rat model demonstrates the multi-modal sensing capability of the sensing apparatus in capturing dynamic variations in pulse oximetry (SpO₂), tissue oximetry (StO₂), pulse intensity (PI), and respiratory intensity (RI), as a ligation on femoral artery is applied. In various embodiments, the sensing apparatus uses spatially resolved pulsation patterns to the localized blood flow rate according to the pulsating phase shift between two photodiode channels. In vivo demonstration on human upper limb upon tourniquet application and physical workout on exercising bike substantiate the continuous recording of localized physiological indicators and highlight the device tolerance to body motion due to anchoring effects from the microneedles. Generally, performance results of the sensing apparatus ratify that various embodiments described herein not only potentially diminish the effort in producing an uninterrupted, precise, and robust screening among high-risk populations of PAD, but also improve the detecting accuracy while receding subject disturbance, compared to existing optical-based soft electronic devices.

### II. Example Operational Architectures and Systems

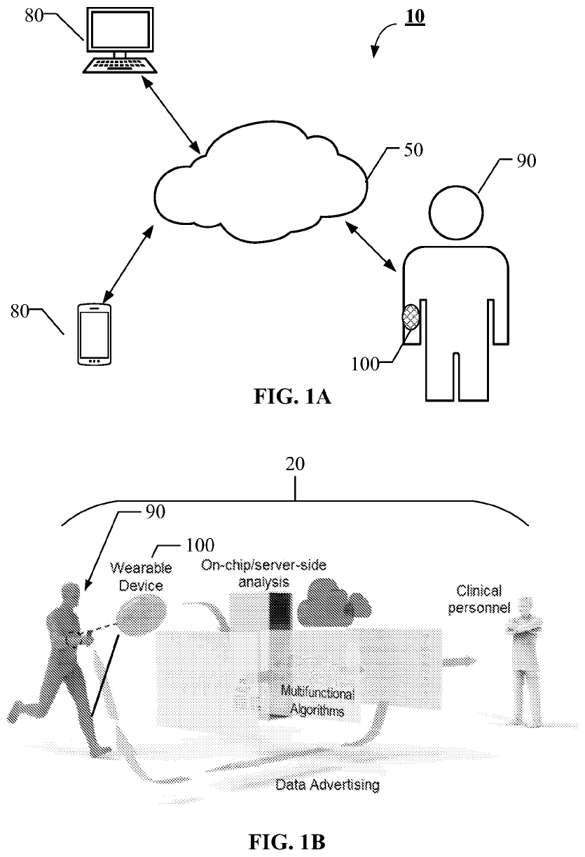

FIG. 1A provides an illustration of a system architecture 10 that may be used in accordance with various embodiments of the disclosure. Here, the architecture 10 includes various components and entities involved in providing deep tissue sensing and health monitoring for a subject 90. In the present disclosure, a subject refers to an entity having tissue layers and physiological characteristics that can be measured using a sensing apparatus in accordance with some embodiments of the present disclosure. Examples of a subject may include, but are not limited to, a human patient, an experimental tissue sample, an animal, and/or the like.

As illustrated, the architecture 10 includes a sensing apparatus 100 interfacing with and worn by the subject 90, with the sensing apparatus 100 configured for deep tissue sensing via a plurality of waveguiding microneedles. The sensing apparatus 100 may include an on-device and on-board device (e.g., a control module) which may perform various signal pre-processing and signal processing operations with collected deep tissue data (such as, but not limited to, sensing data). Further, the sensing apparatus 100 may be configured to, via its on-board device, wireless transmit data (such as, but not limited to, sensing data) to external devices 80 via a network 50. As such, the subject 90, while wearing the sensing apparatus 100, is not limited in movement by the sensing apparatus 100; that is, the sensing apparatus 100 enables wearable and wireless use.

In various embodiments, the external devices 80 may include various devices used to monitor the health of the subject 90 via the collected data. In one example, the external devices 80 includes a computer workstation, a mobile device (e.g., a smartphone) associated with and operated by the subject 90 and configured to visualize/display biometric data based at least in part on the data collected by the sensing apparatus 100, one or more clinician devices, a server configured to store collected data and measurements, and/or the like.

In some embodiments, the external devices 80 is configured to receive the sensing data from the sensing apparatus 100 over time for continuous monitoring of the subject, and determine a plurality of physiological measurements associated with the deep tissue of the subject 90 from the sensing data. For example, sensing data may be collected/transmitted at a configurable frequency (e.g., every 5 seconds, every 10 seconds, every minute). In some embodiments, the sensing apparatus 100 may comprise a control module described herein that transmits, via wireless communication (e.g. via the network 50), sensing data and/or the physiological measurements to the external devices 80.

In various embodiments, the system architecture 10 comprises a cloud and/or distributed medical platform 20, an example of which being illustrated in FIG. 1B. In such a cloud and/or distributed medical platform 20, clinician personnel may fully monitor calculated physiological indicators remotely via server-side cloud and/or distributed computing. The cloud and/or distributed medical platform 20 may be used to further disengage both subjects and clinical personnel from care units by facilitating the measuring process and enabling continuous, accurate observation, all as a result of wearable and wireless use of the sensing apparatus 100 on the subject 90.

As noted, the sensing apparatus 100 and one or more external devices 80 may communicate with one another over one or more networks 50. Depending on the embodiment, these networks 50 may comprise any type of network such as a land area network (LAN), wireless land area network (WLAN), wide area network (WAN), metropolitan area network (MAN), wireless communication network, peer-to-peer networks, cellular communication networks, the Internet, and/or combinations thereof. In addition, these networks 50 may comprise any combination of standard communication technologies and protocols. For example, the networks 50 may provide Bluetooth communication between the sensing apparatus 100 and one or more external devices 80. In various examples, communications may be carried over the networks 50 by link technologies such as Ethernet, 802.11, CDMA, 3G, 4G, 5G or digital subscriber line (DSL). Further, the networks 50 may support a plurality of networking protocols, including the hypertext transfer protocol (HTTP), the transmission control protocol/internet protocol (TCP/IP), or the file transfer protocol (FTP), and the data transferred over the networks 50 may be encrypted using technologies such as, for example, transport layer security (TLS), secure sockets layer (SSL), and internet protocol security (IPsec). Those skilled in the field of the present disclosure will recognize FIGS. 1A and 1B represent possible configurations of system architectures, and that variations are possible with respect to the protocols, facilities, components, technologies, and equipment used.

### III. Example Components and Physical Configurations

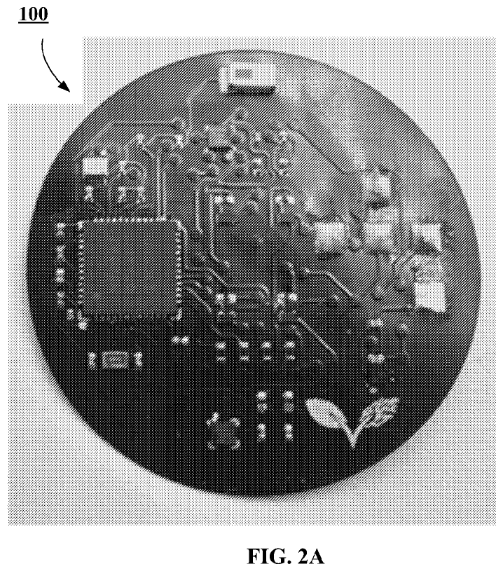

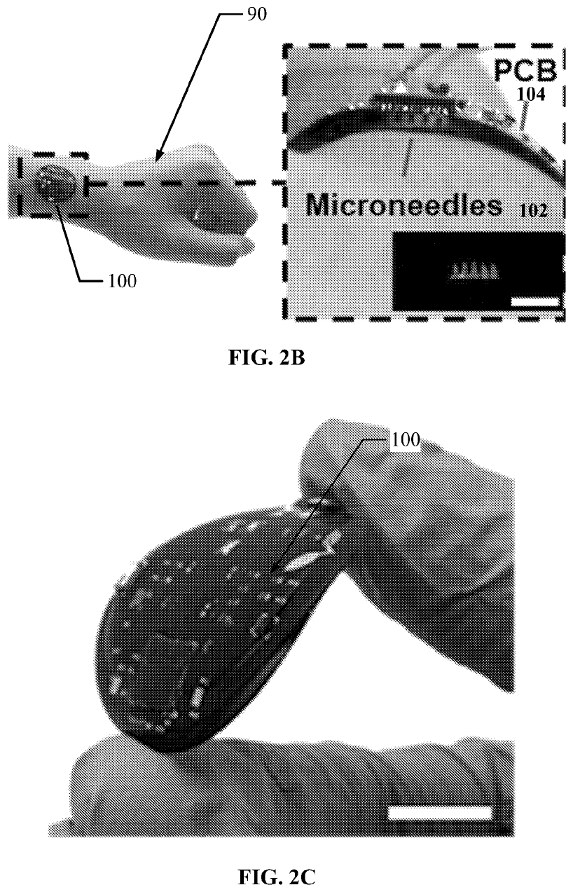

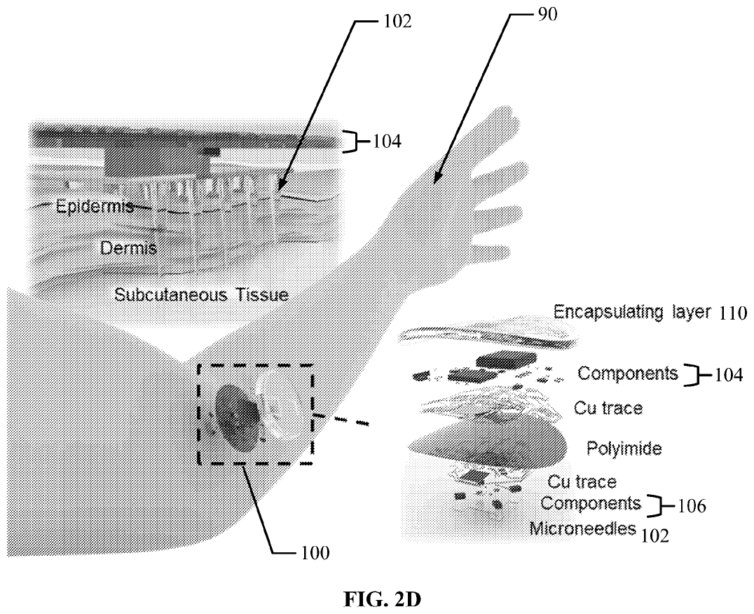

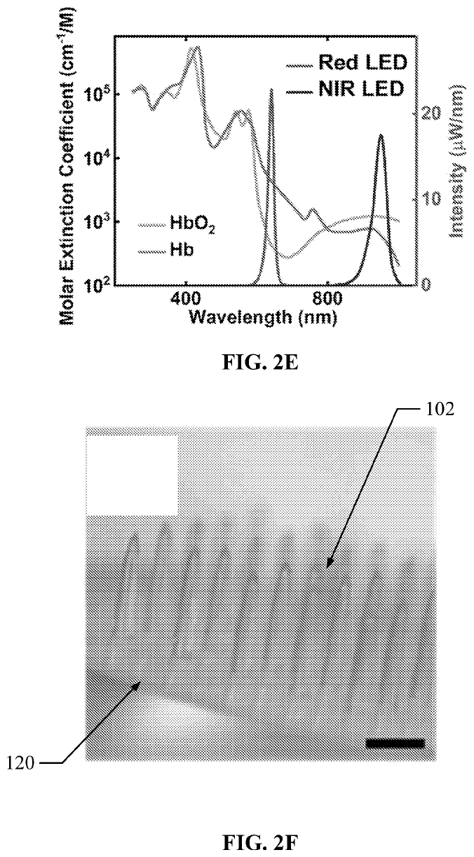

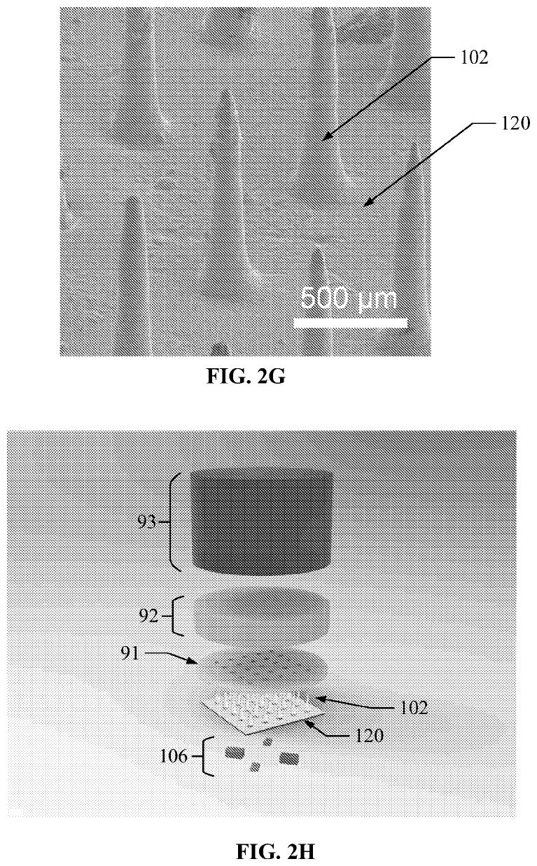

As discussed, various embodiments of the present disclosure provide a sensing apparatus configured for wearable and wireless deep tissue sensing via waveguiding microneedles. According to some example embodiments, the sensing apparatus is embodied by a wearable patch configured for optical/light-based sensing. FIG. 2A illustrates an example top view of such a sensing apparatus 100. As shown in FIG. 2A, the sensing apparatus 100, or the wearable patch, comprises various electronic components for an on-device control module for operating waveform generators and detectors, collecting and processing data, and/or advertising and transmitting data.

FIG. 2B illustrates one example wearable use of the sensing apparatus 100 in which the sensing apparatus 100 or patch interfaces with the skin at a peripheral limb of the subject 90 (e.g., the subject's wrist in the illustrated embodiment). As shown in the inset view of FIG. 2B, the sensing apparatus 100 may be relatively thin and planar with a plurality of microneedles 102 positioned on a subject-interfacing face of the sensing apparatus 100 (for example, on a skin-interfacing portion of a base layer of the sensing apparatus 100). In its wearable use, the microneedles 102 are oriented towards and may extend into the subject 90 (for example, extend into at least a dermal depth and/or a subcutaneous depth of the subject). Meanwhile, a body of the sensing apparatus 100 may house its control module 104 and various other electronic components, which may be embodied by and implemented via a flexible printed circuit board (PCB), for example. As shown, the body of the sensing apparatus 100 including the control module 104 remains positioned external to the subject's body during wearable use. Accordingly, the sensing apparatus 100 is minimally invasive, as the microneedles 102 minimally penetrate into the subject 90. In various embodiments, the microneedles 102 provide an anchoring effect for the sensing apparatus 100 and reduce unwanted shifting, vibrating, or other motions of the sensing apparatus 100 in wearable use. In some examples, the sensing apparatus 100 may include adhesive materials, coatings, and/or the like to further secure its skin interface with the subject 90.

In various embodiments, the sensing apparatus 100 as a wearable patch is configured with mechanical flexibility, as demonstrated in FIG. 2B and FIG. 2C. As such, the sensing apparatus 100 may conform at least to an extent to the skin surface of the subject 90. Conformation of the sensing apparatus 100 may further secure the skin interface with the subject 90 and improves overall quality in the collected data. In various embodiments, the sensing apparatus 100 is configured with a minimal bending radius of at least approximately 50 mm, at least approximately 30 mm, at least approximately 25 mm, or at least approximately 20 mm.

In various embodiments, the wearable wireless sensing apparatus comprises at least three main parts that may be arranged in a stacking fashion. FIG. 2D provides an exploded view of various stacked components that constitute the three main parts of the sensing apparatus 100. A top part or layer of the sensing apparatus 100 houses the control module 104, which may be interconnected with flexible filaments of electrically conductive material (e.g., copper) on a flexible polymeric substrate to enable various operations to be performed, such as generation of physiological measurements associated with deep tissue(s) of the subject and/or wireless communication of data. Examples of the physiological measurements may be selected from the group consisting of at least one of tissue oximetry measurements, pulse oximetry measures, heart pulsation measurements, respiratory measurements, volume measurements, and/or plethysmographic measurements. In various embodiments, the control module 104 is configured for Bluetooth communication and may wirelessly communicate data in a continuous and/or real-time manner according to a Bluetooth Low Energy (BLE) protocol. As one representative non-limiting example, the control module 104 may include one or more Bluetooth-capable modules, SoCs, development boards, and/or the like for wireless communication capabilities. In various embodiments, the control module 104 comprises a core microcontroller and peripheral components to control and operate sensing modules configured to generate and detect sensing wave signals. In various embodiments, the control module 104 may control power, duty cycle, sampling frequency, and/or other parameters of sensing wave signals to satisfy longevity and battery life objectives. For instance, the control module 104 may add a five minute delay after a five-second measurement to increase operating time of the sensing apparatus to over 1300 hours, in some examples. The core microcontroller may be further configured to perform various data pre-processing and data processing operations.

For operation of the control module 104 and other various electronic components, the sensing apparatus 100 may comprise a battery or similar power supply. In various embodiments, the sensing apparatus 100 uses a rechargeable battery to power the entire sensing apparatus 100. For example, a 150 mAh rechargeable Li-ion battery may be integrated to ensure operation at full power (e.g., 25.2 mW, 20% duty cycle) for at least 22 hours of use, as demonstrated in experimental results included herein.

In various embodiments, the top layer and the control module 104 may be covered with a clear encapsulation layer 110 that conformally covers the control module 104 and other electronic components/connections to prevent contact with sweat, biofluids, and/or the like for improved safety and longevity. In one or more example embodiments, the clear encapsulation layer is made of polydimethylsiloxane and has a thickness of approximately 0.25 mm to approximately 2 mm, approximately 0.5 mm to approximately 1.5 mm, or approximately 0.75 mm to approximately 1.25 mm.

A middle part or layer of the sensing apparatus 100 features a collection of sensing modules 106, which include waveform generators and waveform detectors generally. This layer is also referred to as a “sensing layer.” The sensing modules are electronically connected with the control module 104, and in some examples, may be integrated with the control module 104 in one shared flexible PCB. For optical/light-based sensing applications, the sensing modules 106 may specifically include LEDs embodying the waveform generators and configured to generate light wave signals of specific wavelengths, and photodiodes embodying the waveform detectors and configured to detecting light wave signals (e.g., reflected light wave signals) in a range of spectral bandwidth. For example, in one or more example embodiments, the sensing apparatus 100 comprises two LEDs configured for emission of red light at a peak wavelength of between approximately 620 nm and approximately 750 nm, between approximately 625 nm and approximately 700 nm, or between approximately 630 nm and approximately 640 nm (thereby emitting light signals that include visible red light); two LEDs configured for emission of near-infrared light at a peak wavelength of greater than approximately 800 nm, greater than approximately 900 nm, or between approximately 930 nm and approximately 970 nm (thereby emitting light signals that include near-infrared signals); and four photodiodes configured for detection in a bandwidth between approximately 350 and approximately 1070 nm, for example. To support the miniaturization and compact size of the sensing apparatus 100 in minimizing obstruction and inconvenience in wearable use, the red LEDS may be 0.65 mm×0.35 mm×0.2 mm or of similar scale, the near-infrared LEDS may be 1.0 mm×0.5 mm×0.45 mm or of similar scale, and the photodiodes may be 2 mm×1.25 mm×0.85 mm or of similar scale, in one or more example embodiments. In various embodiments, the waveform generators and/or the waveform detectors are less than 1 mm³, less than 4 mm³, less than 5 mm³, or less than 10 mm³.

The use of red and near-infrared sensing wave signals makes the sensing apparatus 100 applicable to oximetry-related measurements and sensing. FIG. 2E illustrates the emission spectra of example red LEDS and near-infrared LEDS as well as the absorption spectra of oxyhemoglobin and deoxyhemoglobin. Measures of the absorption of emitted red and near-infrared sensing wave signals may inform on relative concentrations of oxyhemoglobin and deoxyhemoglobin, which can then be extended to various oximetry-related measurements.

The third main part of the sensing apparatus 100 includes arrays of microneedles 102 configured to be biocompatible and to have waveguiding properties (e.g., transparent for optical/light waveguiding). In various embodiments, the microneedles 102 are attached to a base face or a base layer of the sensing apparatus 100. In some embodiments, the sensing layer described above is positioned above the base layer 120.

In some embodiments, the base layer 120 is configured to interface with a skin surface of a subject. For example, the base layer 120 and the sensing layer described above form a flexible substrate configured to conform to contours of the skin surface of the subject. As the skin surface may not be perfectly planar, the sensing apparatus 100 is configured to adapt and replicate the contours and planar geometry of the skin surface while interfacing/attached.

In some embodiments, the microneedles 102 are attached to a skin-interfacing portion of the base layer 120 and oriented to extend past a skin surface of the subject into at least a dermal depth and/or a subcutaneous depth of the subject. Generally, the microneedles 102 may be thin. For example, the microneedles 102 may have a height of approximately 0.5 mm to approximately 3 mm, approximately 0.5 mm to approximately 5 mm, approximately 1 mm to approximately 8 mm, or less than approximately 10 mm, while having a needle base diameter of approximately 250 μm to approximately 550 μm, approximately 300 μm to approximately 500 μm, or approximately 350 μm to approximately 450 μm. FIG. 2F provides a perspective view of an array of microneedles 102 that are attached to a base layer 120 of the sensing apparatus 100. FIG. 2G provides an SEM image of at least a portion of an array of microneedles (for example, at least a portion of the array of microneedles 102 that are attached to the base layer 120 as described above).

While the description above provides example sizes and images of microneedles in accordance with various embodiments of the present disclosure, it is noted that the scope of the present disclosure is not limited to the examples above.

In various embodiments, the array of microneedles 102 are made of PLGA at each needle tip/body and polyvinyl alcohol (PVA) at the base layer 120. In various embodiments, the microneedles 102 are arranged along/across the base layer of the sensing apparatus 100 in a pattern or layout. In some example embodiments, the microneedles 102 are arranged along/across the base layer with a spacing distance of less than approximately 250 μm, less than approximately 300 μm, less than approximately 400 μm, less than approximately 500 μm, or less than approximately 550 μm. At least some of the microneedles 102 are arranged along/across the base layer to be aligned with the waveform generators and the waveform detectors positioned above or opposite the base layer. In some examples, the microneedles 102 may be separate components that are attached to the base layer via adhesive materials that may not impinge upon or obstruct the waveguiding properties of the microneedles 102, such as a UV curing optical adhesive. As discussed, the microneedles 102 are minimally invasive and penetrate a portion of skin tissue and/or fatty tissue of the subject 90 to a shallow depth during wearable use. FIG. 2H illustrates an exploded view including the microneedles 102 and tissue layers of a subject 90. FIG. 2H specifically illustrates the microneedles 102 being arranged along a base layer 120 of the sensing apparatus 100, with sensing modules 106 being positioned opposite the base layer 120 with respect to the microneedles 102. As shown in FIG. 2H, the microneedles 102 may be configured to penetrate a shallow depth into the subject 90, for example through a portion of a surface skin layer 91 (e.g., an epidermis) of the subject 90. Accordingly, deeper layers such as a fatty tissue layer 92 and a blood layer 93 are not directly penetrated by the microneedles 102. However, due to waveguiding properties of the microneedles 102, sensing wave signals generated via the sensing modules 106 may propagate through the depths significantly deeper than the depths of the microneedles 102. For example, the sensing wave signals may propagate to and/or through the blood layer 93 despite the microneedles 102 only extending to and superficially penetrating the surface skin layer 91. The microneedles 102 may comprise or be comprised of biocompatible material with waveguiding properties. For example, the microneedles 102 may be constructed from heterogeneous integration of PLGA and PVA, serve as waveguiding channels which extends wave propagation by minimizing scattering and absorption along the guiding pathways to effectively increase penetration depth of sensing wave signals (e.g., light signals) inside tissue. As described above, the light signals include, but are not limited to, visible red light signals and near-infrared signals.

For example, the microneedles 102 are configured to waveguide the wave signals into a deep tissue of the subject. In the present disclosure, deep tissue refers to biological tissue located below attenuating surface layers. In some embodiments, a deep tissue defines a domain or a sensing field within which regions of interest for sensing and measurement are located. For example, an example deep tissue may comprise deep tissue layers in humans that may comprise, such as but not limited to, epidermis, dermis, subcutaneous tissue, fascia and/or muscle.

As described above, the sensing apparatus is secured to a subject via penetration and anchoring of the microneedles 102 into the subject. In some example embodiments, the sensing apparatus may include adhesive material that enhances the microneedle attachment/interfacing modality.

### IV. Example Operations

FIG. 3 provides a schematic of an example computing entity 300 that may be used in accordance with various embodiments of the present disclosure to perform example operations described herein. In particular, the computing entity 300 may be configured to perform various example operations described herein to control and operate sensing modules 106 of the sensing apparatus 100 and/or to pre-process/process collected deep tissue data. For example, the computing entity 300 may be embodied by the control module 104 of the sensing apparatus 100 to perform various example operations locally at the sensing apparatus 100. The computing entity 300 may be embodied by one or more external devices 80 to remotely acquire collected data from the sensing apparatus 100 via wireless communication and to perform various example operations with the acquired data (e.g., as part of a cloud and/or distributed medical platform 20).

In general, the terms computing entity, entity, device, and/or similar words used herein interchangeably may refer to, for example, one or more computers, computing entities, desktop computers, mobile phones, tablets, phablets, notebooks, laptops, distributed systems, items/devices, terminals, servers or server networks, blades, gateways, switches, processing devices, processing entities, set-top boxes, relays, routers, network access points, base stations, the like, and/or any combination of devices or entities adapted to perform the functions, operations, and/or processes described herein. Such functions, operations, and/or processes may include, for example, transmitting, receiving, operating on, processing, displaying, storing, determining, creating/generating, monitoring, evaluating, comparing, and/or similar terms used herein interchangeably. In one embodiment, these functions, operations, and/or processes can be performed on data, content, information, and/or similar terms used herein interchangeably.

Although illustrated as a single computing entity, those of ordinary skill in the field should appreciate that the computing entity 300 shown in FIG. 3 may be embodied as a plurality of computing entities, tools, and/or the like operating collectively to perform one or more processes, methods, and/or steps. As just one non-limiting example, the computing entity 300 may comprise a plurality of individual data tools, each of which may perform specified tasks and/or processes.

Depending on the embodiment, the computing entity 300 may include one or more network and/or communications interfaces 320 for communicating with various computing entities, such as by communicating data, content, information, and/or similar terms used herein interchangeably that can be transmitted, received, operated on, processed, displayed, stored, and/or the like. Thus, in certain embodiments, the computing entity 300 may be configured to receive data from one or more data sources and/or devices as well as receive data indicative of input, for example, from a device.

The networks used for communicating may include, but are not limited to, any one or a combination of different types of suitable communications networks such as, for example, cable networks, public networks (e.g., the Internet), private networks (e.g., frame-relay networks), wireless networks, cellular networks, telephone networks (e.g., a public switched telephone network), or any other suitable private and/or public networks. Further, the networks may have any suitable communication range associated therewith and may include, for example, global networks (e.g., the Internet), MANs, WANs, LANs, or PANs. In addition, the networks may include any type of medium over which network traffic may be carried including, but not limited to, coaxial cable, twisted-pair wire, optical fiber, a hybrid fiber coaxial (HFC) medium, microwave terrestrial transceivers, radio frequency communication mediums, satellite communication mediums, or any combination thereof, as well as a variety of network devices and computing platforms provided by network providers or other entities.

Accordingly, such communication may be executed using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. Similarly, the computing entity 300 may be configured to communicate via wireless external communication networks using any of a variety of protocols, such as general packet radio service (GPRS), Universal Mobile Telecommunications System (UMTS), Code Division Multiple Access 2000 (CDMA2000), CDMA2000 1× (1×RTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), 5G New Radio (5G NR), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.11 (Wi-Fi), Wi-Fi Direct, 802.16 (WiMAX), ultra-wideband (UWB), infrared (IR) protocols, near field communication (NFC) protocols, Wibree, Bluetooth protocols (e.g., Bluetooth Low Energy, or BLE), wireless universal serial bus (USB) protocols, and/or any other wireless protocol. The computing entity 300 may use such protocols and standards to communicate using Border Gateway Protocol (BGP), Dynamic Host Configuration Protocol (DHCP), Domain Name System (DNS), File Transfer Protocol (FTP), Hypertext Transfer Protocol (HTTP), HTTP over TLS/SSL/Secure, Internet Message Access Protocol (IMAP), Network Time Protocol (NTP), Simple Mail Transfer Protocol (SMTP), Telnet, Transport Layer Security (TLS), Secure Sockets Layer (SSL), Internet Protocol (IP), Transmission Control Protocol (TCP), User Datagram Protocol (UDP), Datagram Congestion Control Protocol (DCCP), Stream Control Transmission Protocol (SCTP), HyperText Markup Language (HTML), and/or the like.

In addition, in various embodiments, the computing entity 300 includes or is in communication with one or more processing elements 305 (also referred to as processors, processing circuitry, and/or similar terms used herein interchangeably) that communicate with other elements within the computing entity 300 via a bus, for example, or network connection. As will be understood, the processing element 305 may be embodied in several different ways. For example, the processing element 305 may be embodied as one or more complex programmable logic devices (CPLDs), microprocessors, multi-core processors, coprocessing entities, application-specific instruction-set processors (ASIPs), and/or controllers. Further, the processing element 305 may be embodied as one or more other processing devices or circuitry. The term circuitry may refer to an entirely hardware embodiment or a combination of hardware and computer program products. Thus, the processing element 305 may be embodied as integrated circuits, application specific integrated circuits (ASICs), field programmable gate arrays (FPGAs), programmable logic arrays (PLAs), hardware accelerators, other circuitry, and/or the like.

As will therefore be understood, the processing element 305 may be configured for a particular use or configured to execute instructions stored in volatile or non-volatile media or otherwise accessible to the processing element 305. As such, whether configured by hardware, computer program products, or a combination thereof, the processing element 305 may be capable of performing steps or operations according to embodiments of the present disclosure when configured accordingly.

In various embodiments, the computing entity 300 may include or be in communication with non-volatile media (also referred to as non-volatile storage, memory, memory storage, memory circuitry and/or similar terms used herein interchangeably). For instance, the non-volatile storage or memory may include one or more non-volatile storage or non-volatile memory media 310 such as hard disks, ROM, PROM, EPROM, EEPROM, flash memory, MMCs, SD memory cards, Memory Sticks, CBRAM, PRAM, FeRAM, RRAM, SONOS, racetrack memory, and/or the like. As will be recognized, the non-volatile storage or non-volatile memory media 310 may store files, databases, database instances, database management system entities, images, data, applications, programs, program modules, scripts, source code, object code, byte code, compiled code, interpreted code, machine code, executable instructions, and/or the like. The term database, database instance, database management system entity, and/or similar terms used herein interchangeably and in a general sense to refer to a structured or unstructured collection of information/data that is stored in a computer-readable storage medium.

In particular embodiments, the non-volatile memory media 310 may also be embodied as a data storage device or devices, as a separate database server or servers, or as a combination of data storage devices and separate database servers. Further, in some embodiments, the non-volatile memory media 310 may be embodied as a distributed repository such that some of the stored information/data is stored centrally in a location within the system and other information/data is stored in one or more remote locations. Alternatively, in some embodiments, the distributed repository may be distributed over a plurality of remote storage locations only. As already discussed, various embodiments contemplated herein use data storage in which some or all the information/data required for various embodiments of the disclosure may be stored.

In various embodiments, the computing entity 300 may further include or be in communication with volatile media (also referred to as volatile storage, memory, memory storage, memory circuitry and/or similar terms used herein interchangeably). For instance, the volatile storage or memory may also include one or more volatile storage or volatile memory media 315 as described above, such as RAM, DRAM, SRAM, FPM DRAM, EDO DRAM, SDRAM, DDR SDRAM, DDR2 SDRAM, DDR3 SDRAM, RDRAM, RIMM, DIMM, SIMM, VRAM, cache memory, register memory, and/or the like.

As will be recognized, the volatile storage or volatile memory media 315 may be used to store at least portions of the databases, database instances, database management system entities, data, images, applications, programs, program modules, scripts, source code, object code, byte code, compiled code, interpreted code, machine code, executable instructions, and/or the like being executed by, for example, the processing element 305. Thus, the databases, database instances, database management system entities, data, images, applications, programs, program modules, scripts, source code, object code, byte code, compiled code, interpreted code, machine code, executable instructions, and/or the like may be used to control certain aspects of the operation of the computing entity 300 with the assistance of the processing element 305 and operating system.

As will be appreciated, one or more of the computing entity's components may be located remotely from other computing entity components, such as in a distributed system. Furthermore, one or more of the components may be aggregated, and additional components performing functions described herein may be included in the computing entity 300. Thus, the computing entity 300 can be adapted to accommodate a variety of needs and circumstances.

Embodiments of the present disclosure may be implemented in various ways, including as computer program products that comprise articles of manufacture. Such computer program products may include one or more software components including, for example, software objects, methods, data structures, and/or the like. A software component may be coded in any of a variety of programming languages. An illustrative programming language may be a lower-level programming language such as an assembly language associated with a particular hardware architecture and/or operating system platform. A software component comprising assembly language instructions may require conversion into executable machine code by an assembler prior to execution by the hardware architecture and/or platform. Another example programming language may be a higher-level programming language that may be portable across multiple architectures. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query or search language, and/or a report writing language. In one or more example embodiments, a software component comprising instructions in one of the foregoing examples of programming languages may be executed directly by an operating system or other software component without having to be first transformed into another form. A software component may be stored as a file or other data storage construct. Software components of a similar type or functionally related may be stored together such as, for example, in a particular directory, folder, or library. Software components may be static (e.g., pre-established or fixed) or dynamic (e.g., created or modified at the time of execution).

A computer program product may include a non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media (including volatile and non-volatile media).

In one embodiment, a non-volatile computer-readable storage medium may include a floppy disk, flexible disk, hard disk, solid-state storage (SSS) (e.g., a solid state drive (SSD), solid state card (SSC), solid state module (SSM)), enterprise flash drive, magnetic tape, or any other non-transitory magnetic medium, and/or the like. A non-volatile computer-readable storage medium may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile computer-readable storage medium may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

In one embodiment, a volatile computer-readable storage medium may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media may be substituted for or used in addition to the computer-readable storage media described above.

As should be appreciated, various embodiments of the present disclosure may also be implemented as methods, apparatus, systems, computing devices, computing entities, and/or the like. As such, embodiments of the present disclosure may take the form of a data structure, apparatus, system, computing device, computing entity, and/or the like executing instructions stored on a computer-readable storage medium to perform certain steps or operations. Thus, embodiments of the present disclosure may also take the form of an entirely hardware embodiment, an entirely computer program product embodiment, and/or an embodiment that comprises a combination of computer program products and hardware performing certain steps or operations.

Embodiments of the present disclosure are described with reference to example operations, steps, processes, blocks, and/or the like. Thus, it should be understood that each operation, step, process, block, and/or the like may be implemented in the form of a computer program product, an entirely hardware embodiment, a combination of hardware and computer program products, and/or apparatus, systems, computing devices, computing entities, and/or the like carrying out instructions, operations, steps, and similar words used interchangeably (e.g., the executable instructions, instructions for execution, program code, and/or the like) on a computer-readable storage medium for execution. For example, retrieval, loading, and execution of code may be performed sequentially such that one instruction is retrieved, loaded, and executed at a time. In some example embodiments, retrieval, loading, and/or execution may be performed in parallel such that multiple instructions are retrieved, loaded, and/or executed together. Thus, such embodiments can produce specifically configured machines performing the steps or operations specified in the block diagrams and flowchart illustrations. Accordingly, the block diagrams and flowchart illustrations support various combinations of embodiments for performing the specified instructions, operations, or steps.

Various example operations that may be performed by the computing entity 300 generally include pre-processing and/or processing of collected data (such as, but not limited to, sensing data), generating multiple physiological measures, transmitting collected data and/or physiological measures, displaying physiological measures, and/or the like. For example, the computing entity 300 may perform various example operations with a graphic tool (e.g., configured based on the PySerial library, configured based on the matplotlib library) to provide direct visualization of the sensing data and diagnostic analysis. Meanwhile, the computing entity 300 may perform various example operations to train, implement, and/or use machine learning models to denoise collected data and/or generate physiological measures, in various embodiments. For example, the physiological measurements described above can be determined from the sensing data using one or more machine learning models trained at least to reduce noise in the sensing data.

In the present disclosure, training machine learning models generally refers to the generation/configuration of machine learning models. In neural network machine learning models, training involves adjustment and modification of nodal parameters (e.g., weights, biases) to obtain desired outputs from the models. “Trained” in past tense or as an adjective may describe a model that has been trained to a certain level of performance (e.g., accuracy) in a validation stage, for example.

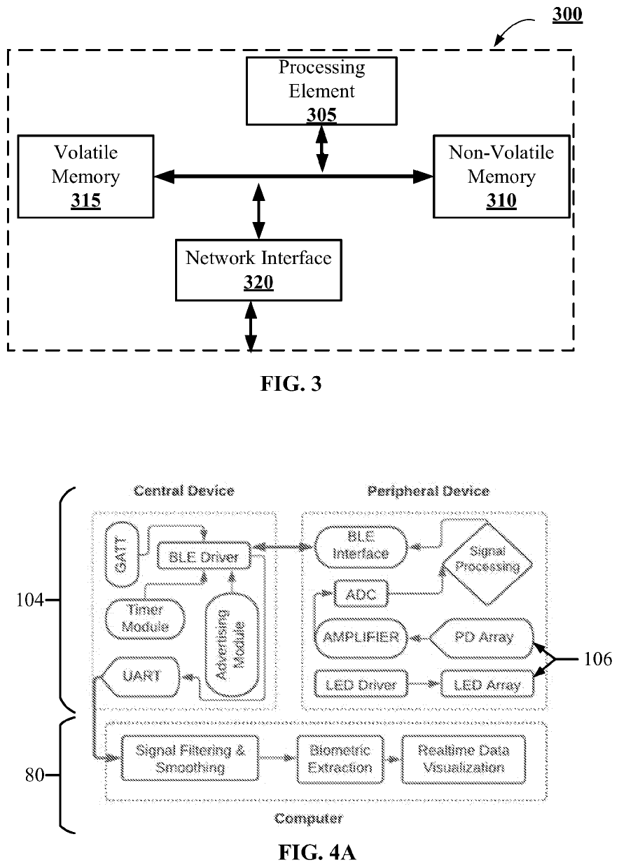

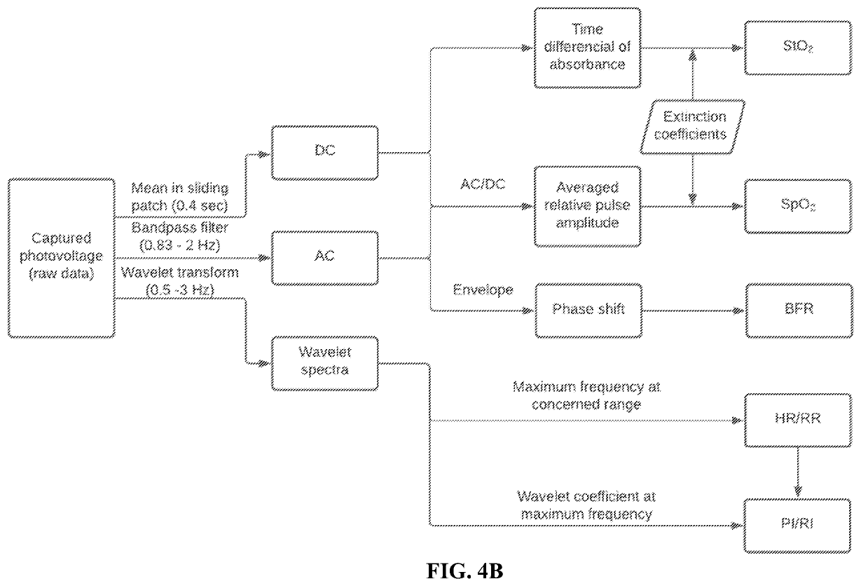

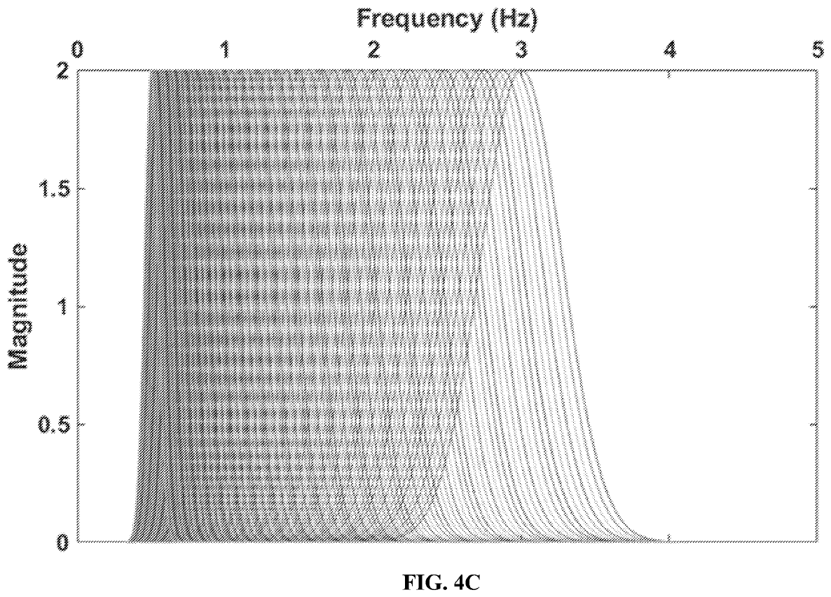

FIGS. 4A and 4B provide example block diagrams and flowcharts that describe various example operations that may be performed by the computing entity 300 and/or other computing entities. In FIG. 4A, sensing modules 106 are operated to emit and detect optical/light wave signals. In some example embodiments, signal processing may be performed locally with the collected data. In various embodiments, the sensing modules 106 may then communicate with the control module 104 and/or a central device via Bluetooth Low Energy (BLE) communication. The control module 104 may then communicate the processed data to an external device 80, which may perform further operations including signal filtering & smoothing, biometric extraction, and real-time data visualization, according to some example embodiments.

In FIG. 4B, a block diagram illustrating various example operations related to generation of physiological measures from collected data is provided in an optical/light-based application. As shown, raw photovoltage data is collected at one or more sensing modules 106 (specifically, photodiodes), and the raw photovoltage data is processed to generate sensing data and determine multiple different physiological measures simultaneously or near-simultaneously based on the sensing data. In various embodiments, the raw photovoltage data is acquired in four channels and two wavelengths at a sampling frequency of 100 Hz, for example.

Referring first to the generation of heart rate (HR), respiratory rate (RR), pulse intensity (PI), and respiratory intensity (RI) physiological measures, the computing entity 300 may be configured to perform wavelet transform operations to process the raw photovoltage data, as illustrated in FIG. 4B. In particular, the continuous wavelet transform (CWT) is adapted to analyze the raw data. In some example embodiments, a Morse wavelet family with a skewness parameter γ=3 and a time-bandwidth product P²=120 is defined to serve as the basis of the transform. An example wavelet family used to generate HR, RR, PI, and RI physiological measures is shown in FIG. 4C. In various embodiments, the wavelet family is constrained to a frequency range of approximately 0.25 Hz to approximately 6 Hz, approximately 0.35 Hz to approximately 5 Hz, approximately 0.5 Hz to approximately 3 Hz, or approximately 1 Hz to approximately 2 Hz to avoid high-frequency noises and baseline drifts, and 48 increments are created within a frequency octave. The wavelet coefficients with respect to time are then given by the wavelet transform, defined in Equation 1.

$${W}_{s} ( t ) = {\int }_{- ∞}^{∞} \frac{1}{s} {ψ}^{*} ( \frac{u - t}{s} ) x ( u ) du = \frac{1}{2 π} {\int }_{- ∞}^{∞} {Ψ}^{*} ( s ω ) X ( ω ) {e}^{i ω t} d ω \qquad ( Equation 1 )$$

In Equation 1, s represents the increment in the frequency space,

$$Ψ ( ω ) = {\int }_{- ∞}^{∞} ψ ( t ) {e}^{- i ω t}$$

represents the Fourier transform of the prepared wavelet ψ(t), and X(ω) represents the Fourier transform of the raw data x(u). In various embodiments, a steady HR of the subject 90 and corresponding frequency increment (s_{HR}) may be obtained (e.g., via a separate and independent oximeter, via manual techniques, via profile data associated with the subject 90), and the frequency increment is used to generate the HR physiological measurement. Specifically, the HR physiological measurement for the subject 90 may be defined as HR(t)=s_{HR}ω. Meanwhile, the PI physiological measurement for a steady subject (expecting significant change in pulse amplitude but a constant HR) may be defined as the change of the wavelet coefficient with time at so, PI(t)=Wₛ_{_{HR}}(t). Similarly, RR and RI physiological measurements may be defined as RR(t)=s_{RR}ω and RI(t)=Wₛ_{_{HR}}(t), respectively.

On the other hand, HR and PI physiological measurements for a moving subject (expecting slight change in pulse amplitude but significant change in HR) may be generated by the strongest frequency component in the whole range, specifically and respectively,

$$HR ( t ) = {arg max}_{s} ( {W}_{s} ( t ) ) ω and PI ( t ) = {max}_{s} ( {W}_{s} ( t ) ) .$$

RR and RI physiological measurements for a moving subject may be determined by the max frequency component near the steady frequency s_{RR}ω. Specifically,

$$RR ( t ) = {max}_{s ∈ ( {s}_{R R} - 5 , {s}_{R R} + 5 )} ( {W}_{s} ( t ) ) ω and RI ( t ) = {max}_{s ∈ ( {s}_{R R} - 5 , {s}_{R R} + 5 )} ( {W}_{s} ( t ) ) .$$

In various embodiments, the computing entity 300 is configured to calculate both steady and moving physiological measurements for HR, PI, RR, and RI. In various embodiments, the computing entity 300 may receive movement data for the subject 90, such as from gyroscopic and/or accelerometer components of the sensing apparatus 100 or of other devices associated with the subject 90, and may accordingly determine whether to generate steady physiological measurements or moving physiological measurements based at least in part on processing the movement data, for example.

FIG. 4B additionally illustrates generation of SpO₂ physiological measurements based at least in part on processing the raw photovoltage raw data. In various embodiments, generation and analysis of SpO₂ data utilizes electrical signal processing, and in some examples, may rely on visual indications of pulse and oximetry techniques. That is, generation of SpO₂ data may be based at least in part on optical/light-based sensing involving the red and near-infrared waveform generators and detectors, as previously discussed.

As indicated, band-pass filtering may be performed on the acquired raw photovoltage data to extract heart rate pulsing frequency components (AC(t)). In various embodiments, the bandwidth of the band-pass filter is based at least in part on subject characteristics. For example, a bandwidth for band-pass filtering may be defined between approximately 0.83 Hz and approximately 2 Hz if the subject is a human, generally, while the bandwidth may be defined between approximately 3 Hz and approximately 6 Hz if the subject is an animal. Subject demographic data and historical data may generally be accessed to determine the bandwidth for the band-pass filter, in some embodiments. In one or more example embodiments, a second order digital Butterworth filter is used for the band-pass filtering.

In various embodiments, a moving-mean function (configured to return the mean value of the data over a sliding interval, for example, 0.4 seconds) may be used to exclude fluctuations in the filtered data due to factors unrelated to pulsation. Use of the moving-mean function yields DC components, or DC(t). Then, in various embodiments, the relative signal intensity Si(t) of pulsations in the data is generated as the ratio between the primary pulse frequency AC(t) and the denoised data

$$D C ( t ) : Si ( t ) = \frac{A C ( t )}{D C ( t )} .$$

A relative signal intensity is generated for each of red and near-infrared data.

In various embodiments, an envelope function, using a discrete Fourier transform, may be used on the data to identify upper and lower envelopes, or respectively, U(t) and L(t). Generally, the upper and lower envelopes characterize the minimum and maximum points for each pulse, respectively. From this, a modulation amplitude can be generated: A(t)=U(t)−L(t).

Then, a ratio between red and near-infrared components at every data sample is evaluated in a moving integration window of 15 seconds according to Equation 2.

$$R ( t ) = {\sum }_{15 s} \frac{log ( {A ( t )}_{R} + 1 )}{log ( {A ( t )}_{I R} + 1 )} \qquad ( Equation 2 )$$

In various embodiments, Equation 3 may then be used to generate a SpO₂ physiological measure. Equation 3 is derived through photo-diffusion theory and modified Beer-Lambert Law to express the relationship between arterial blood saturation SpO₂ and the ratio defined in Equation 2.

$${Sp O}_{2} ( t ) = \frac{{ε}_{H b}^{R E D} D P {F}_{R / IR} - {ε}_{H b}^{N I R} R ( t )}{( {ε}_{H b}^{R E D} - {ε}_{H b {O}_{2}}^{R E D} ) D P {F}_{R / IR} + ( {ε}_{H b {O}_{2}}^{N I R} - {ε}_{H b}^{N I R} ) R ( t )} \times 100 \qquad ( Equation 3 )$$

In Equation 3,

$${ε}_{H b {O}_{2}}^{R E D} and {ε}_{H b}^{R E D}$$

represent the extinction coefficients of oxyhemoglobin and deoxyhemoglobin for the red wavelength and

$${ε}_{H b {O}_{2}}^{NIR} and {ε}_{H b}^{NIR}$$

are those for the near infra-red wavelength. DPF_{R/IR} represents the ratio between the differential pathlength factors for the two relevant wavelengths. In some examples, the extinction coefficients may be defined as follows:

$${ε}_{H b {O}_{z}}^{R E D} = 0.011 {mm}^{- 1} , {ε}_{H b}^{R E D} = 0.1 \; 06 {mm}^{- 1} ,$$

$${ε}_{H b {O}_{2}}^{N I R} = 0.0 \; 28 {mm}^{- 1} , {ε}_{H b}^{NIR} = 0.0 \; 18 {mm}^{- 1} .$$

FIG. 4B further illustrates generation of tissue oximetry (StO₂) physiological measurements. In various embodiments, assessment of StO₂ is based at least in part on the measurement of the backscattered light intensity I of the input light beam with light intensity I₀. In human tissue, the light attenuation is mainly contributed by absorption and scattering. The absorption behavior can be described by the definition of absorbance,

$$A = {log}_{10} ( \frac{{I}_{0}}{I} ) ,$$

and the Beer-Lambert law A=εcd, where ε represents the specific molar extinction coefficient of the chromophore, c represents the molar concentration of the chromophore, and d represents the traveling distance of the light beam. The scattering behavior can be again described by the modified Beer-Lambert law, which includes the effect of absorption and scattering. The modified Beer-Lambert law is shown in Equation 4.

$$A = {log}_{10} ( \frac{{I}_{0}}{I} ) = {\sum }_{i} {ε}_{i} {c}_{i} d B + G \qquad ( Equation 4 )$$

In Equation 4, εᵢ and cᵢ represent the extinction coefficient and concentration of the i^{th} chromophore, respectively. B represents the dimensionless differential path length factor that calculates the scattering distance from the travel distance d, and G represents the unknown factor that gives the total light losses results from the scattering process which is independent on time. In human tissue, two chromophores are concerned: oxyhemoglobin (HbO₂) and deoxyhemoglobin (Hb). In some example embodiments, the hemoglobin concentration in human tissue may be assumed to be 16 g/dl and the steady thenar StO₂ at 75%. Since the molar mass of hemoglobin is 64500 g/mol, it can be found that the steady concentrations for oxyhemoglobin and deoxyhemoglobin are 1.86 mM and 0.62 mM, respectively. In addition to the initial concentration of two types of hemoglobin, the extinction coefficients of two types of hemoglobin at two wavelengths were the same as stated in the SpO₂ analysis. Therefore, the contribution of the two chromophores for the total absorbance of the red and NIR wavelengths can be generated according to Equation 5.

$$[ \begin{matrix}{A}_{R E D} \\ {A}_{N I R}\end{matrix} ] = d B [ \begin{matrix}{ε}_{Hb}^{RED} & {ε}_{{HbO}_{2}}^{RED} \\ {ε}_{Hb}^{NIR} & {ε}_{{HbO}_{2}}^{NIR}\end{matrix} ] [ \begin{matrix}{c}_{Hb} \\ {c}_{{HbO}_{2}}\end{matrix} ] + [ \begin{matrix}{G}_{R E D} \\ {G}_{N I R}\end{matrix} ] \qquad ( Equation 5 )$$

Here, both G terms can be eliminated by measuring the absorbance at two times, t₁ and t₂, thereby arriving at Equation 6, in which Δ denotes the change of the variable in the period of (t₂-t₁).

$$[ \begin{matrix}Δ {A}_{R E D} \\ Δ {A}_{N I R}\end{matrix} ] = d B [ \begin{matrix}{ε}_{Hb}^{RED} & {ε}_{{HbO}_{2}}^{RED} \\ {ε}_{Hb}^{NIR} & {ε}_{{HbO}_{2}}^{NIR}\end{matrix} ] [ \begin{matrix}Δ {c}_{H b} \\ Δ {c}_{{HbO}_{2}}\end{matrix} ] \qquad ( Equation 6 )$$

The change of the concentrations of both chromophores can then be generated from Equation 7, and StO₂ is then generated by the accumulative increment described from Equation 7, as shown in in Equation 8.

$$[ \begin{matrix}Δ {c}_{H b} \\ Δ {c}_{{HbO}_{2}}\end{matrix} ] = {\frac{1}{d B} [ \begin{matrix}{ε}_{Hb}^{RED} & {ε}_{{HbO}_{2}}^{RED} \\ {ε}_{Hb}^{NIR} & {ε}_{{HbO}_{2}}^{NIR}\end{matrix} ]}^{- 1} [ \begin{matrix}Δ {A}_{R E D} \\ Δ {A}_{N I R}\end{matrix} ] \qquad ( Equation 7 )$$

$${St O}_{2} ( t ) = \frac{{c}_{H b {O}_{2}} ( t = 0 ) + {Σ}_{t} Δ {c}_{H b {O}_{2}} ( {t}_{i} )}{\begin{matrix}{c}_{H b {O}_{2}} ( t = 0 ) + {c}_{H b} ( t = 0 ) + \\ {Σ}_{i} Δ {c}_{H b {O}_{2}} ( {t}_{i} ) + {Σ}_{i} Δ {c}_{H b} ( {t}_{i} )\end{matrix}} \qquad ( Equation 8 )$$

In Equation 8, Δc_{HbO}_{₂}(tᵢ)=c_{HbO}_{₂}(tᵢ)−c_{Hbo}_{₂}(tᵢ₋₁), and Δc_{Hb}(tᵢ)=c_{Hb}(tᵢ)−c_{Hb}(tᵢ₋₁).

FIG. 4B additionally describes generation of blood flow rate (BFR) physiological measurements. In various embodiments, the computing entity 300 is configured to assess and generate BFR physiological measurements through investigating the phase shift between the signal detected by a pair of waveform detectors (e.g., photodiodes, ultrasonic detectors) whose connection line is parallel to the orientation of the focused vessel. For the signal captured by both waveform detectors, the corresponding moments of the local peaks in the pulses is extracted and arranged in time sequence. Subtraction of the moments of the peaks captured in the downstream waveform detector (m_{d}(t)) by the peaks captured in the upstream waveform detector (mᵤ(t)) yields the phase shift, or Δm(t)=m_{d}(t)−mᵤ(t), based at least in part on which BFR physiological measurements can be generated, in various embodiments.

As discussed, various embodiments include the use of machine learning models in processing data collected via the sensing apparatus 100 to address various technical challenges. A large issue especially for wearable devices is signal processing. Many naïve approaches, such as deterministic models, enable a trade-off between fitness and smoothness, thus making such models less sensitive to outliers within the training set. However, in time-series data with large numbers of outliers, many of these models often are degraded. Additionally, most naïve models cannot accurately approximate missing data, resulting in poor performance on especially wireless-communication biosensors.

Thus, to enable real-time analysis of sensing information collected from the wearable patch, various embodiments include example operations for implementing machine learning models and algorithms for signal processing, which facilitates an intelligent denoising process of measurements that balances the trade-off between fitness and smoothness, compared with traditional deterministic models. In various embodiments, machine learning models based at least in part on autoencoder concepts and techniques enable the intelligent recreation of entire datasets by first compressing (encoding) a given dataset into a latent space, and then reconstructing (decoding) the dataset from that latent representation. This strategy enables much of the noise to be removed through a probabilistic model, accomplished by neural networks.

Specifically, a raw sensing signal (for example, sensing data described above) is first fed through a neural network that attempts to encode the input data to the smallest possible latent representation; then a secondary neural network attempts to decode the latent representation to a representation that mimics the original as closely as possible through optimizer algorithms, such as the Adam Optimizer algorithm for example. Finally, reconstruction loss (e.g., Huber Loss) is calculated and used to tune the model further through backpropagation.

In various embodiments, the machine learning model may be trained by generating a simulated dataset through addition of gaussian noise to a pulse signal, specifically an ideal pulse waveform. If model training time should be reduced, the signal may be down-sampled (e.g., from 2 kHz to 500 Hz). The resulting samples may then be separated into training and test datasets (e.g., a 70/30 split, an 80/20 split, and/or the like). The model can be fit over a plurality of epochs and iterations.

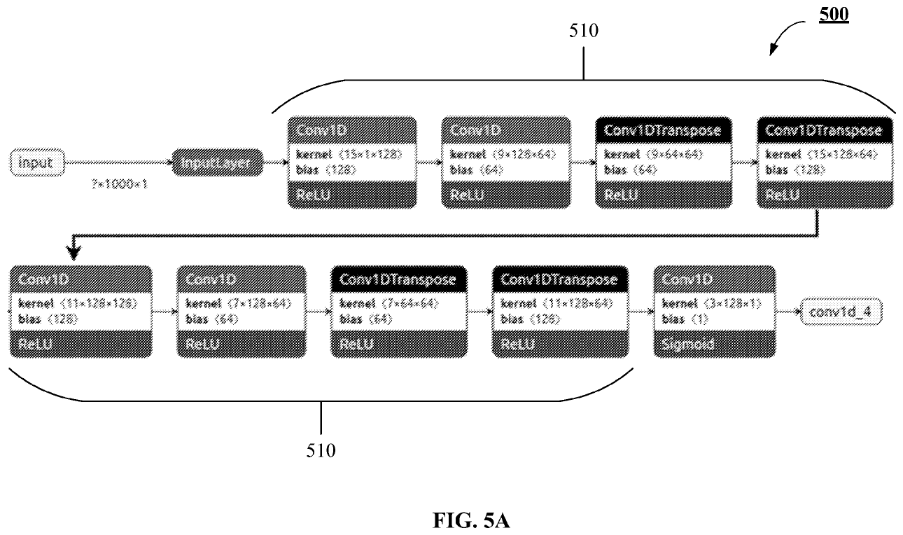

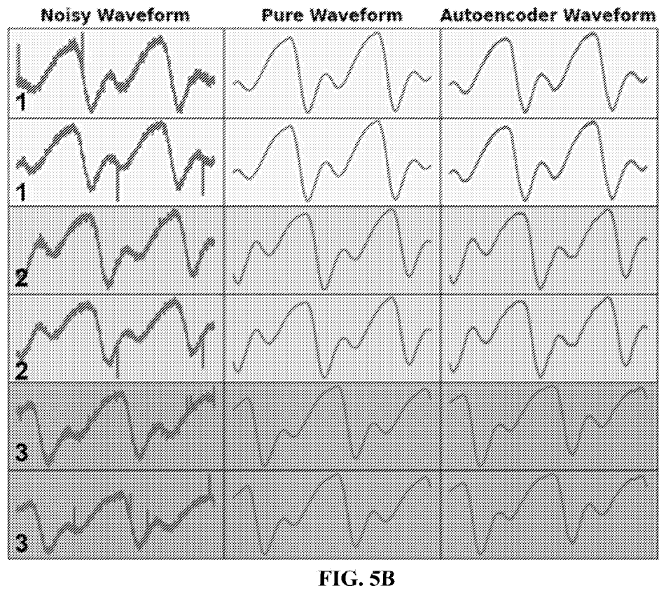

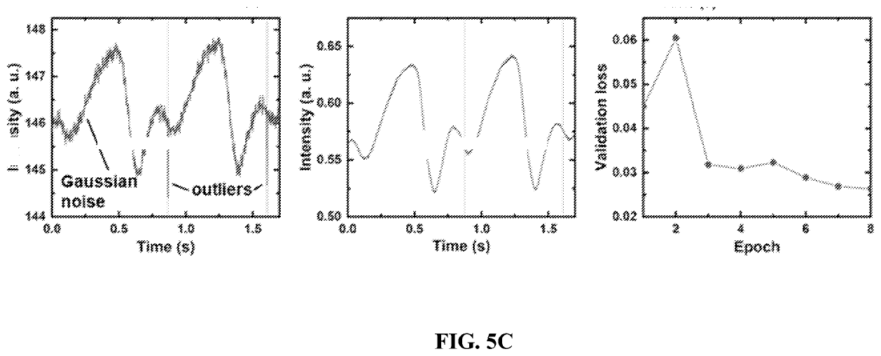

An example architecture for a machine learning model 500 used in conjunction with various embodiments described herein is shown in FIG. 5A. As illustrated, the machine learning model 500 may include two encoder-decoder modules 510. In the illustrated embodiment, the first module's encoder and decoder layers are made of two convolutional neural networks (CNNs) each, with the outermost layers having 128 filters and a kernel size of 15, and the innermost layers having 64 filters and a kernel size of 9. In the illustrated embodiment, the second module uses the same architecture, but with the outermost layers having a kernel size of 11 as opposed to 15, and the innermost having a kernel size of 7 instead of 9.

FIG. 5B illustrates three different groups of pulsation samples (each containing two pulses) that are experimentally processed and denoised using an autoencoder-based machine learning model. Each pair of noisy waveforms is created by adding both Gaussian noises and random outliers to a pulsation signal (shown as pure waveforms) measured at the identical environment. The result provided via the autoencoder-based machine learning model is demonstrated as the autoencoder waveforms in the third illustrated column. As shown, autoencoder-based machine learning models are reasonably effective in denoising pulsed signals and in recreating pure waveforms, in various examples.

FIG. 5C illustrates additional experimental results relating to the function and performance of an autoencoder-based machine learning model. FIG. 5C shows a noisy waveform that is captured by the waveform detectors of the sensing apparatus 100 in the left-most plot, with the noisy data including Gaussian noise and outliers. Using the autoencoder-based machine learning model, the noisy waveform is reduced into the denoised waveform shown in the middle plot, which is significantly better than the original one. It is demonstrated that both the Gaussian noise and the outliers are largely removed. Meanwhile, the right-most plot of FIG. 5C demonstrates the validation loss reduction with more training epochs for the autoencoder-based machine learning model.

While various embodiments discussed herein relate to autoencoder-based machine learning models, other machine learning models configured for denoising and signal processing may be used. For example, recurrent neural networks (RNNs), deep neural networks (DNNs), generative adversarial networks (GANs), generative networks, attention networks, graph convolutional neural networks, and/or the like may be implemented in example embodiments. With different machine learning models, different learning techniques may be used, ranging from supervised learning to unsupervised learning to reduce reliance on ground truth values to train with the noise contrastive estimation. In some example embodiments, unsupervised learning for machine learning models may be advantageous as the machine learning models are trained primarily using real-time and actual data, as opposed to simulated data.

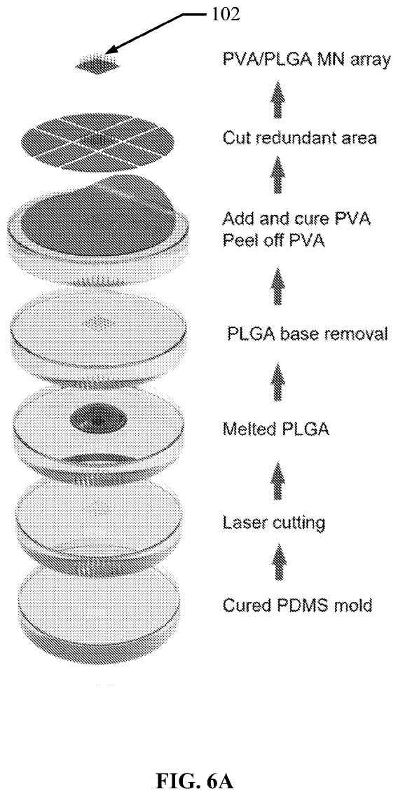

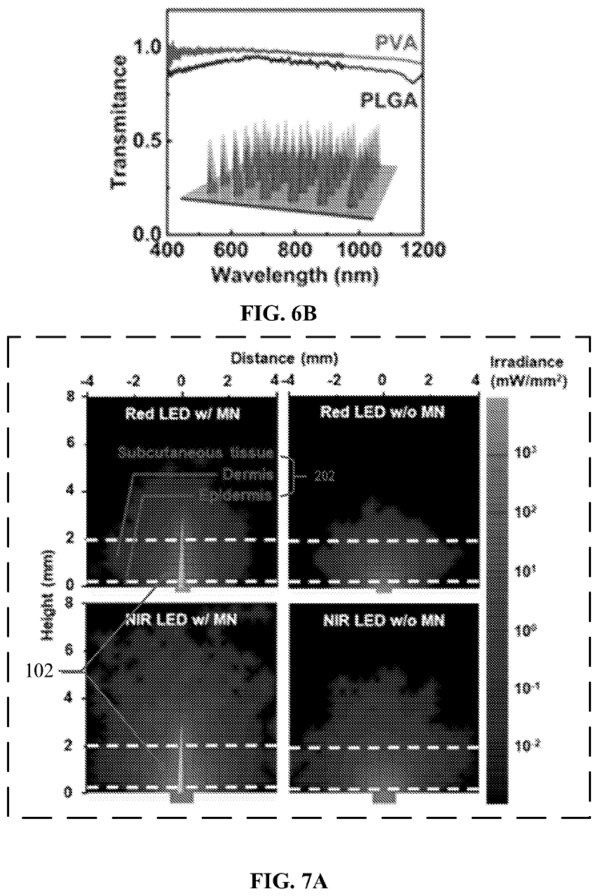

Further to the above-described concepts relating to operations for generating physiological measurements, the present disclosure further provides operations related to the fabrication and manufacturing of components of the sensing apparatus 100. For instance, FIG. 6A illustrates an example process for fabricating the array of microneedles 102 for a sensing apparatus 100. As discussed, the microneedles 102 may be comprised of PLGA through the needle tips and may include PVA at the needle bases and/or may be attached to a PVA base layer, in various embodiments.

As shown in FIG. 6A, the PLGA tips of the microneedles 102 may be first prepared through the following steps. It will be understood that, while the following steps include example material amounts, temperatures, time periods, and/or the like, each of these parameters can be adjusted to produce different configurations of microneedles 102 as needed. 50 grams of poly(dimethyl siloxane) (PDMS) is fully cured in a petri dish at 60° C. for 30 minutes with a curing agent (e.g., mass ratio 1:10). Then, a negative microneedle mold is patterned on the cured PDMS by an ultraviolet (UV) laser ablation system. The microneedle mold is produced and configured according to the desired length, diameter, and spacing for the microneedles 102. The depth of the needle well of the mold can be adjusted for different precise microneedle lengths as needed based at least in part by altering a mark loop parameter of the UV laser. The resolution of the needle tip is increased by decreasing the line distance of the cut pattern. After completely immersing the molds in ethyl acetate and sonicating for 5 minutes, a thin layer of release agent is sprayed on to the mold. Pure PLGA pellets are then densely placed on top of the negative microneedle mold. After that, the entire PLGA-covered mold is heated at a temperature of 230° C. and a gauge pressure of 700 mmHg for 15 minutes or until the melted PLGA stops generating bubbles, whichever took longer. Thereafter, the PLGA residue outside the negative microneedle molds is removed while it was still hot, and the mold is then allowed to quench at −20° C. for 20 minutes to have the PLGA fully cured.

Next, the PVA base is prepared starting with 10 mL of 10 wt. % PVA aqueous solution. The solution is added to the above-mentioned petri dish on the PDMS mold with PLGA tips and heated at a temperature of 50° C. and a gauge pressure at 700 mmHg. The vacuum is vented after 2 hours while the products were heated for another 2 days at 50° C. to fully cure and harden the PVA base. Finally, the PVA/PLGA microneedle array is carefully demolded, and the redundant PVA base was removed.

As a general process then, fabrication of the microneedles 102 may comprise definition (e.g., laser patterning) of a negative microneedle mold, filling the negative microneedle mold with PLGA, curing the PLGA, adding PVA above the negative microneedle mold and the cured PLGA, curing the PVA, and removing the fabricated PVA/PLGA microneedle array.

In various embodiments, the microneedle design uses a strategy of heterogenous integration based on two mechanically distinct biopolymers, PLGA and PVA, where the former with higher elastic modulus (e.g., 2.0 GPa) serves as the needle bodies and the latter with lower elastic modulus (e.g., 707.9 MPa) serves as the base layer, in order to realize optical/light-based waveguiding properties for the microneedles 102. FIG. 6B provides the measured absorption spectra of a PVA film and a PLGA film, respectively, highlighting the high transparency at targeted wavelength regime (400 to 1200 nm) for optical/light-based sensing. The high optical transparency and sufficient refractive index (e.g., 1.47 for both materials) at the concerned wavelength regime (e.g., approximately 639 nm for red sensing wave signals and approximately 950 nm for near-infrared sensing wave signals) of PLGA and PVA validate the waveguiding capability of the integrated microneedles inside biological tissues.

Generally, depending on the sensing application and modality, the microneedles 102 are composed of materials having low resistivity for the sensing wave signals, and in various embodiments, microneedle materials are further required to be biocompatible. In some examples, the microneedles 102 may be hollow, formed of biocompatible metallic materials (e.g., stainless steel, titanium), and/or the like for waveguiding ultrasonic sensing wave signals.

### V. Example Implementations

### 1. Electronic Module Configurations

In various embodiments, a BLE-enabled microcontroller may be used to embody the control module 104 to perform waveform generator (e.g., LED) activation and waveform detector (e.g., photodiode) data sampling. The microcontroller was fit in a recommended peripheral circuit layer as shown in FIG. 4A. Except for the required components, the microcontroller was also configured in a way that eight General Purpose Input/Output (GPIO) channels on the microcontroller were enabled. Amongst them, one of the quad-channel set was configured as GPIO input, and were connected to four photodiodes via two transimpedance amplifiers with a feedback resistor of 1 MΩ. Acting as transimpedance amplifiers, their output was converted to digital signal by the successive approximation analog-to-digital converter (SAADC) in the SoC, as also shown in FIG. 4A.

The other quad-channel set were configured as GPIO output and enabled the individual actuation of the four LEDs digitally. To prevent excessive heating of the device, the duty cycle of each LED was set to 20%, with each period lasting 10 milliseconds (thus ensuring any LEDs will be on for no longer than 2 milliseconds at a time). In various embodiments, the duty cycle can be dynamically controlled by the microcontroller based at least in part on detected temperature data. An individual analog-to-digital (ADC) capture event was comprised of a burst sample (256 samples per event) and averaged prior to transmission to the central device. All data sampled by GPIO input channels were advertised via the BLE protocol, and decoded by the BLE driver on the central device.

Three sets of data are transmitted per event: i) ADC captures for when the red LEDs are on, ii) the IR LEDs are on, and iii) when no LEDs are on. The presence of the blank capture allows for ambient noise deduction from the RED and IR signals, thus improving performance of the sensing apparatus 100. By subtracting from each ADC measurement of LED light absorption the immediately following ambient noise, variation in environmental light lasting longer than 2 milliseconds can be removed, thus reducing the impact of environmental variability. Additionally, since the calculation of pulse oximetry (SpO₂) is based upon the AC and DC components of absorption of red and infrared light, the removal of ambient light prevents the overestimation of the DC components, thus making the resulting components more reflective of simply the reflectance absorption of the red and infrared light, not the environmental light.

To improve stability of the Bluetooth connection, and minimize packet loss and corruption, each packet contained 16 ADC samples, and three pieces of metadata—total number of samples, timer period, and buffer length. The samples are ordered as follows: four samples during red LED activation, four samples of ambient light, four samples during IR LED activation, and four samples of ambient light, where each sample corresponds to the ADC capture from a one of four photodiodes. By choosing a relatively small packet size, the impact of a single mis-represented bit is lower, thereby lessening the impact of packet corruption (an issue especially for BLE devices). A two-millisecond delay was instituted between each event of sample collection, thus giving time for burst sampling during each event. Once received by the central device, the central device sends each packet to a computer through a serial port for post-processing using universal asynchronous receiver-transmitter (UART) protocol.

### 2. Deep Tissue Sensing Experimentation

The implementation of one or more arrays of microneedles 102 on the sensing apparatus 100 enables a safe strategy to effectively increase penetration depth inside tissue with enlarged irradiated area and concentrated light field at depth, both of which facilitate the goal of deep tissue sensing via skin. The epidermal layer primarily consists of stratum corneum, stratum lucidum, stratum granulosum, stratum spinosum, and stratum basales, where the heterogeneous compositions of biomolecules and endogenous chromophores (e.g., melanin, hemoglobin, nucleic acids, proteins, bilirubin, and others) generate a strong scattering and extinction effect on the visible and infrared light, making it challenging for deep tissue sensing at this wavelength regime from 400 to 1100 nm.

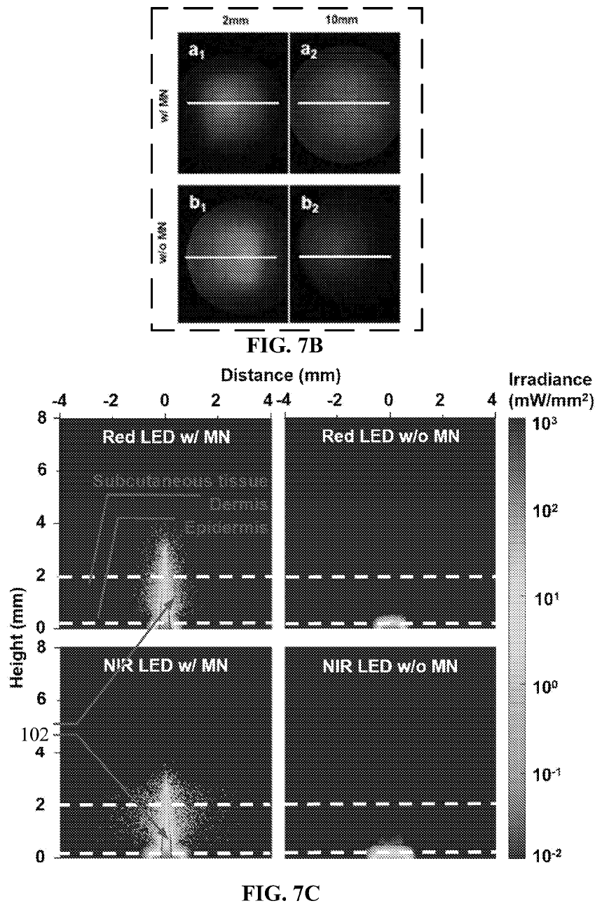

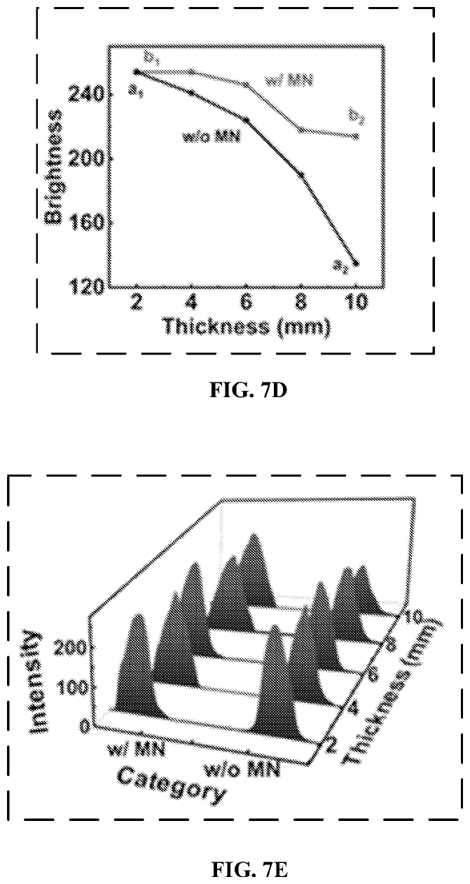

FIG. 7A shows the results of a Monte Carlo simulation to compare the difference in the spatial distributions of illumination in human tissue between devices with and without incorporation of the microneedle waveguide. The incident radiant energy in the range of 10⁻² to 103 mW/mm² at the profile is mapped. Example representative boundaries of the epidermis (e.g., thickness=0.1 mm), dermis (e.g., thickness=1.9 mm), and subcutaneous tissue (e.g., thickness>6 mm) at the profiles are highlighted with the dashed lines in FIG. 7A. An example height and penetration depth of the microneedle 102 into the tissue of 3 mm is used. The illumination profile was extracted from the simulation volume given by the incident radiant energy per unit area at the profile (mW/mm²), which represents the phenomenological optical irradiance.

The four cross-sectional views of the simulation results demonstrates that the 3-mm microneedle situated on the top surface of a LED significantly expands the illuminated area of the red LED (at a ratio of 2.67) and that of the infrared LED (at a ratio of 1.99). The depth of the illuminated tissue increases from 0.75 mm to 4.75 mm for red light, and infrared light from 1 mm to 4 mm for infrared light. FIG. 7B illustrates a corresponding planar view of the illumination or sensing field for the experimental setup of FIG. 7A. With the same illumination emitted from two red LEDs, the one with incorporation of the microneedle arrays exhibited a wider illumination area at 10 mm depth inside an artificial tissue, compared with the other one without incorporation of the microneedle arrays, further confirming the microneedle waveguides facilitate an extended penetration of light inside a highly scattering media of biological tissue. In general, these results demonstrate the augmentation of an optical/light-based sensing field via optically waveguiding microneedles (e.g., transparent PVA/PLGA microneedles), and in various embodiments, the microneedles 102 may be configured with waveguiding properties for other modalities to generate similar sensing field augmentation. For example, the microneedles 102 may be configured with ultrasonic waveguiding properties such that an ultrasonic sensing field is also expanded.

Similarly, FIG. 7C shows another set of results of a Monte Carlo simulation to compare the difference in the spatial distributions of illumination in human tissue between devices with and without incorporation of the microneedle waveguide. In particular, the simulation results are on profiles of the light irradiance, inside skin tissue, of the LEDs (wavelength 660 nm and 950 nm, respectively. The incident radiant energy in the range of 10⁻² to 103 mW/mm² at the profile is mapped. Example representative boundaries of the epidermis (e.g., thickness≈0.1 mm), dermis (e.g., thickness≈1.9 mm), and subcutaneous tissue (e.g., thickness>6 mm) at the profiles are highlighted in FIG. 7C. An example height and penetration depth of the microneedle 102 into the tissue of 3 mm is used. The illumination profile was extracted from the simulation volume given by the incident radiant energy per unit area at the profile (mW/mm²), which represents the phenomenological optical irradiance.

FIGS. 7D and 7E provide quantitative comparisons of the experimental microneedle implementations and non-microneedle implementations in improving light penetration inside an artificial tissue. Specifically, FIG. 7D shows the measured intensity, and 7E shows the spatial distribution of the optical/light sensing field propagated through an artificial tissue at various depths. As highlighted again in FIGS. 7D and 7E, penetration of light is extended via the waveguiding microneedles.

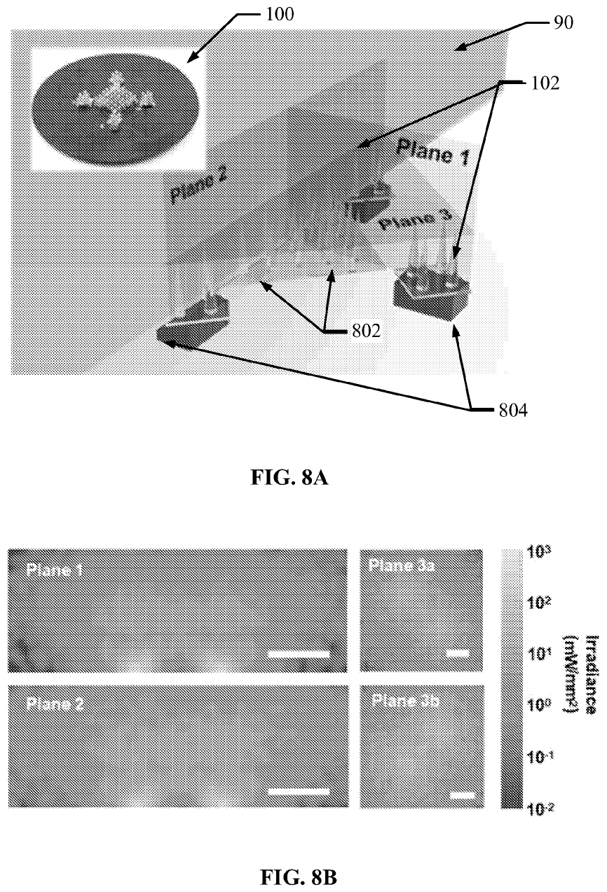

As discussed, the microneedles 102 are arranged in a pattern or configuration across the base layer 120, and in various embodiments, the microneedles 102 are generally aligned with waveform generators and/or waveform detectors positioned opposite of the base layer 120. FIG. 8A illustrates one example design layout of waveform generators 802, waveform detectors 804, and the microneedles 102. In the example layout, the waveform generators 802, embodied by red and near-infrared LEDs, are situated in the center, and the waveform detectors 804, embodied by photodiodes, are situated on four corners surrounding the center, forming an overall cross shape. Accordingly, the example layout further includes one array of microneedles 102 in the center and aligned with the waveform generators 802, as well as individual arrays of microneedles 102 at the corners aligning with the waveform detectors 804 (also shown in the inset of FIG. 8A).

FIG. 8B provides simulation results for the example design layout of the waveform generators 802, waveform detectors 804, and the microneedles 102. The results include cross-sections with respect to three axial planes for an optical/light-based sensing field resulting from operation of the LEDs and the photodiodes. For the simulation, the operation of the LEDs follows a periodic cycle which turns on the red LEDs and the near-infrared LEDs in a sequential fashion at a frequency of 100 Hz and with a duty cycle of 20%. According to the simulation results, the effective volume of illumination (the sensing field) is 33.51 mm³ and 56.11 mm³ for red and near-infrared sensing wave signals, respectively.

### 3. Mechanical, Thermal, And Electrical Property Experimentation

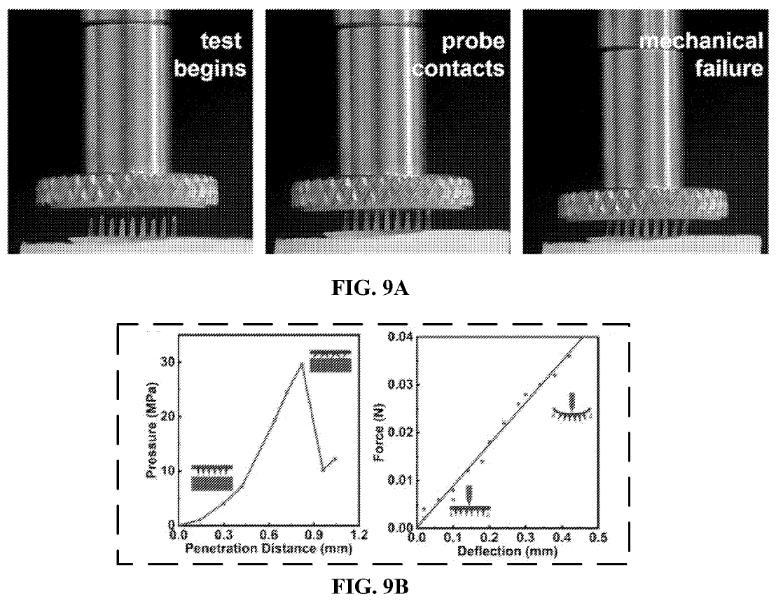

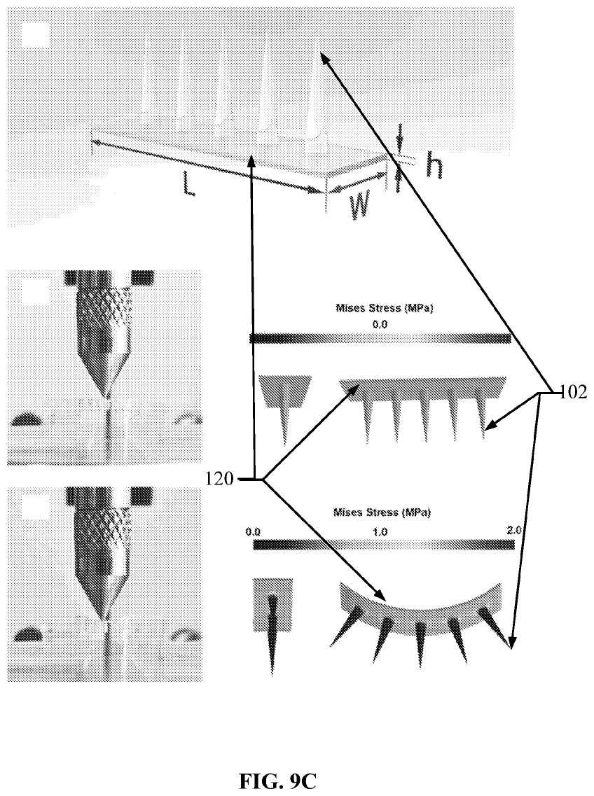

Characterizations on the mechanical, thermal, and electrical properties of the sensing apparatus 100 are experimentally demonstrated and described herein. According to various embodiments, the heterogeneous integration of PLGA and PVA in the microneedles 102 offers a leveraged mechanical property for the sensing apparatus 100 with both sufficient rigidity for facilitating skin penetration and flexibility for tolerating skin deformation. FIG. 9A shows an example experimental procedure for verifying piercing capability of the microneedles 102. In FIG. 9A, a 5-by-5 microneedle array is prepared and tested against a rigid plane. During the test, the substrate (e.g., the base layer 120) of the microneedle array was fixed to the test plane to avoid sliding of the microneedle array. The rigid plane was set to a constant descent speed (e.g., 13.0 mm/min) to sample the elastic force exerted by the microneedle array until failure. The pressure produced by the microneedle array was calculated according to the diameter of the tip area of the microneedles 102 (e.g., 10 μm), and the total contact area is then given by A=25π(d/2)²=1.96×10⁻⁹ m². The maximum pressure before mechanical failure was then 29.6 MPa, as shown in the left-most plot of FIG. 9B. This maximum pressure is significantly higher than what is needed to penetrate human skin (approximately 0.68 MPa). These results demonstrate that the microneedles 102 maintain the structural integrity throughout the penetration process, with no observable damages or deformations to the microneedles.

The bending characteristics of the microneedle array are also experimentally evaluated, as shown in FIG. 9C and the right-most portion of FIG. 9B. The flexural modulus was measured by a 3-point measurement on a sample microneedle array shown in FIG. 9C, with base layer dimensions L=5.6 mm, W=1.2 mm, and h=0.15 mm. The flexural modulus of the sample is given by Equation 9, in which F represents the load force in Newtons (N).

$${E}_{flex} = \frac{{L}^{3} F}{4 W {h}^{3} d} \qquad ( Equation 9 )$$

Equation 9 can be modified into a form in which the modulus can be found by regression analysis of the sampled d-F relation, this form being shown in Equation 10.

$$d = \frac{{L}^{3}}{4 W {h}^{3} {E}_{flex}} F \qquad ( Equation 10 )$$

The right-most portion of FIG. 9B illustrates d-F samples and a linear regression thereof. The linear regression of the d-F samples gave the result d (mm)=AF+B, where A=87.03±2.53 (mm/N), B=(1.55±5.98)×10⁻⁴ (mm). The statistical t-values and p-values for variable A and B were t_{A}=34.40 (p=6.28×10⁻¹<0.05), t_{B}=0.26 (p=0.80>0.05), respectively. Hence, the existence of variable B could be rejected with a 95% confidence level. The flexural modulus E was then calculated by the estimated result A (variation thereof was calculated by the law of propagation of uncertainty,

$$Δ A = \frac{\partial A}{\partial E} \cdot Δ E ) by$$

$${E}_{f l e x} = \frac{{L}^{3}}{4 W {H}^{3} A} = 99.65 \pm 2.89 ( N / {mm}^{2} ) or {E}_{flex} = ( 9.965 \pm 0.289 ) \times {10}^{- 1} ( MPa ) .$$

Thus, as demonstrated, the combination of flexible materials for the sensing apparatus 100, or the microneedle specifically, contributes to high tolerance of mechanical bending with a minimum bending radius of approximately 25 mm, in some examples. This enables conformal lamination onto major locations of human skin. Thus, the heterogenous integration of PVA and PLGA enables the microneedle to adapt itself under the motion of the skin without delamination or damage. FIG. 9C additionally illustrates finite-element analysis (FEA) on stress distribution of the microneedle array under a bending, which indicates no observable stress concentration (maximum 2.0 MPa at the base of the microneedles 102) during a typical bending process of wearing and demonstrates the durability of the microneedle array in response to mechanical bending.

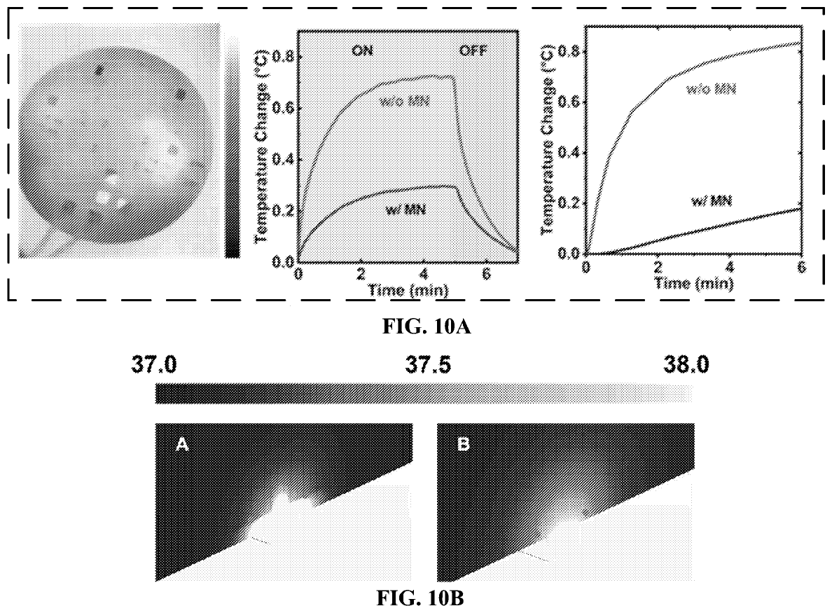

As mentioned, another technical advantage of incorporating microneedles 102 at the sensing interface for the sensing apparatus 100 is that the microneedle array forms a thermal barrier due to the low thermal conductivity of PVA (e.g., approximately 0.205 W/mK), which prevents unwanted transfer of heat into the skin tissue. For example, the base layer and the microneedles of the sensing apparatus are configured to minimize a transfer of ambient heat originating from the one or more waveform generators to the skin surface of the subject, such that the skin surface of the subject may not experience a significant increase in temperature. FIG. 10A compares the thermal characteristics of wearable optical sensors with and without an array of microneedles. The left-most image of FIG. 10A provides a thermal image of a microneedle-incorporated sensing apparatus placed onto a wrist of a user after 15 minutes of normal operation, indicating thermal budget for the wearable device is acceptable for interfacing with the skin and leads to negligible heat transfer to the skin.

The middle plot of FIG. 10A shows that the surface heat release of the microneedle-incorporated sensing apparatus is much lower than that of a microneedle-less sensing apparatus. This further confirms the capability of thermal insulation via the incorporation of microneedle arrays for the sensing apparatus 100.

For these temperature-related investigations, a thermistor was attached to PVA base layer for microneedle-implemented configurations and directly aligned with the LEDs for microneedle-less configurations described by FIG. 10A. Resistance of the thermistor was recorded and converted to temperature:

$$R = {R}_{0} exp [ B ( \frac{1}{T} - \frac{1}{{T}_{0}} ) ] , or \frac{1}{T} = \frac{1}{{T}_{0}} + \frac{1}{B} ln \frac{R}{{R}_{0}} ,$$

where R represents the measured resistance at T, R₀=10kΩ is the resistance measured in room temperature and T₀=25° C., and B represents the thermistor constant. In various embodiments, a sensing apparatus 100 may include one or more thermistor and/or temperature sensing modules and is configured to use them for collection of temperature data during operation and use.

FIG. 10B provides FEA simulations of heat transfer to investigate temperature change along a tissue profile when applying the sensing apparatus 100 on the skin surface. When considering only the effect generated by the sensing apparatus 100 on the skin tissue, the Pennes bioheat equation can be written according to Equation 11.

$$ρ {C}_{p} ( \frac{\partial T}{\partial t} ) + ∇ \cdot ( - k ∇ T ) = {Q}_{e x t} + {φμ}_{a} \qquad ( Equation 11 )$$

In Equation 11, T represents the absolute tissue temperature; t represents the time; ρ and Cₚ represent the mass density and heat capacity of the skin tissue, respectively; and Qₑₓₜ represents the heat generated from external heat sources of the waveform generators (e.g., LEDs) due to power dissipation. With LEDs, the heat associated with light emission was calculated as the product of the light fluence rate φ obtained in the optical simulation and the absorption coefficient μₐ of the skin tissue. The optical and thermal properties appear in Tables 1 and 2. Table 1 provides optical properties of materials used in the simulation, and Table 2 provides thermal properties of the materials, with * denoting approximated values. The skin tissue, sensing apparatus geometry, and the LEDs were modeled using four-node tetrahedral elements. Convergence tests of the mesh size were performed to ensure accuracy. The total number of elements in the models was approximately 260,000.

TABLE 1

| | Absorption coefficient, | Scattering coefficient, |
|---|---|---|
| Material | μₐ [mm⁻¹] | μₛ [mm⁻¹] |
| Epidermis | 0.9 (639 nm) | 5.3 (639 nm) |
| | 0.2 (950 nm) | 3.4 (950 nm) |
| Dermis | 0.054 (639 nm) | 3.2 (639 nm) |
| | 0.052 (950 nm) | 1.4 (950 nm) |
| Subcutaneous | 0.05 (639 nm) | 1.75 (639 nm) |
| fat | 0.03 (950 nm) | 1.25 (950 nm) |
| PVA | 0.00001* | 0.00001* |
| PLGA | 0.00001* | 0.00001* |

TABLE 2

| | Heat | | Thermal |
|---|---|---|---|
| | Capacity, | | conductivity, |
| | cₚ | Density, ρ | K |
| Material | [J/(kg · K)] | [kg/m³] | [W/(m · K)] |
| Skin | 3600 | 1190 | 0.235 |
| Fat | 2348 | 911 | 0.233 |
| PVA | 1010 | 1200 | 0.31 |
| PLGA | 1870 | 1306 | 0.31* |

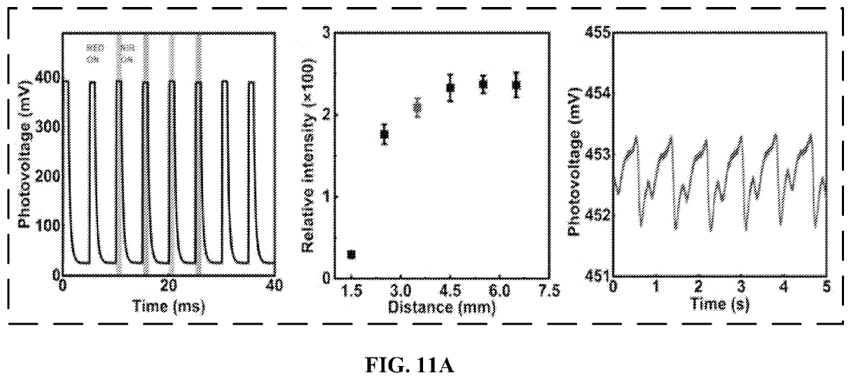

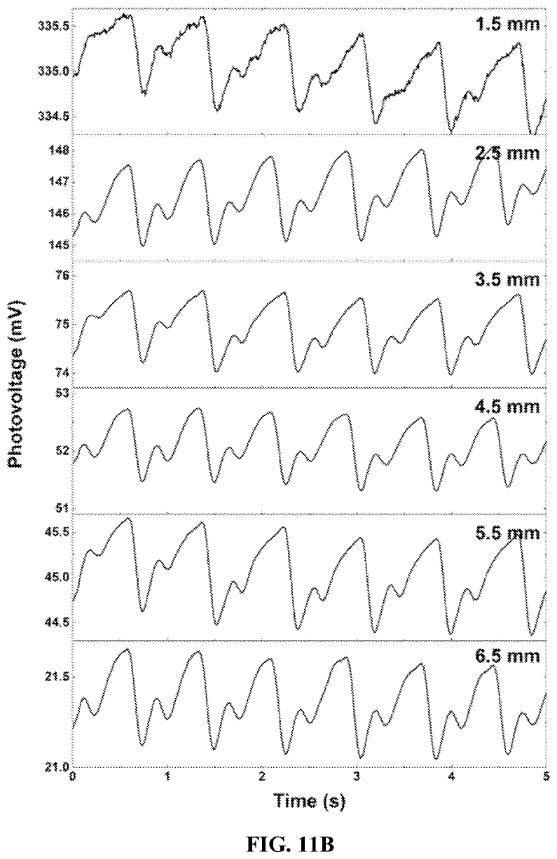

Referring now to FIG. 11A, pulsation patterns and sensing wave signals emitted by the waveform generators 802 (e.g., LEDs) were experimentally configured and tested. The left-most plot of FIG. 11A shows the characteristics of sequential illumination (duty cycle at 20%, frequency at 200 Hz) of red and NIR LEDs with emission intensity tailored to match a comparable illumination volume inside tissue for the two different wavelengths for optimized sensing precision. Furthermore, the spacing between the photodiodes and the LEDs not only determines the sensing field or space but also affects signal-to-noise ratio (SNR) in the collected data. The middle plot of FIG. 11A shows the average relative amplitude of the pulse against the baseline within a 15-second window, indicating that when the distance between LED and photodiode is smaller than 3.5 mm, the relative intensity increases with the space between photodiodes and LEDs. But, at distance 3.5 mm, the relative intensity of signals reaches saturation with minimized SNR, thus providing an optimized sensitivity for the sensing apparatus 100. The right-most plot of FIG. 11A shows the measured pulsation patterns using an example sensing apparatus 100 configured with 3.5 mm distance between adjacent photodiodes and LEDs.

FIG. 11B provides a collection of measured pulsation patterns for different example configurations characterized by variable distances between adjacent photodiodes and LEDs. The example configurations included a series of photodiodes with distances to a center point of a pair of LEDs of 1.5 mm, 2.5 mm, 3.5 mm, 4.5 mm, 5.5 mm, and 6.5 mm. The data processing started with collecting the amplitude of the pulses in a 30-second window, as shown in FIG. 11B. The amplitudes were then averaged over a collection of the pulses within the window. The relative intensity was defined as the average pulse amplitude divided by the average photovoltage within the window and is shown in FIG. 11A.

### 4. In Vivo Experimentation

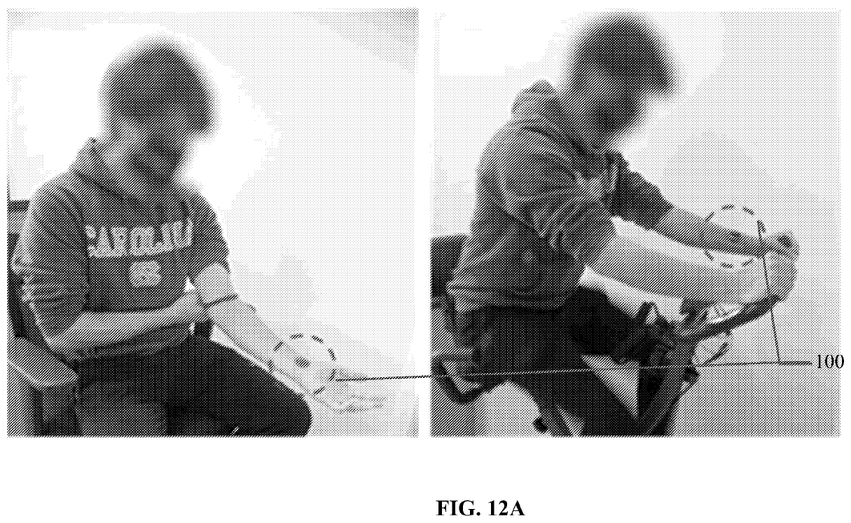

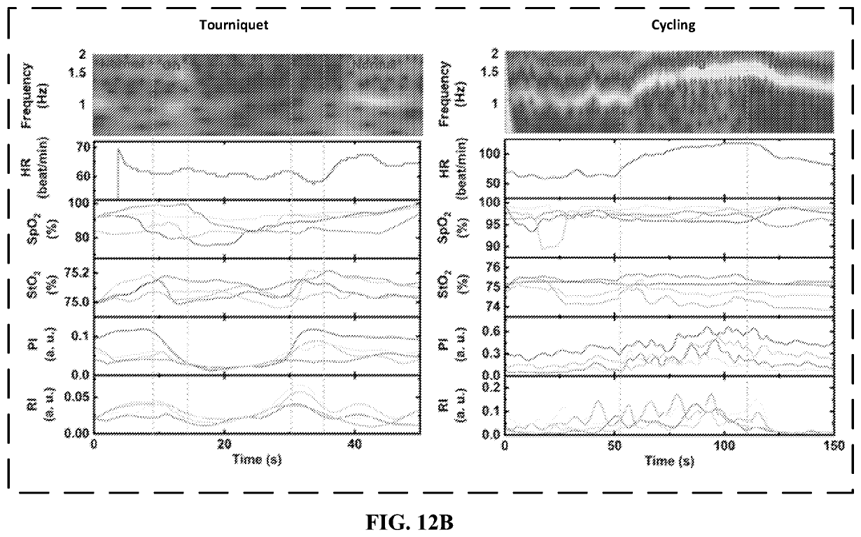

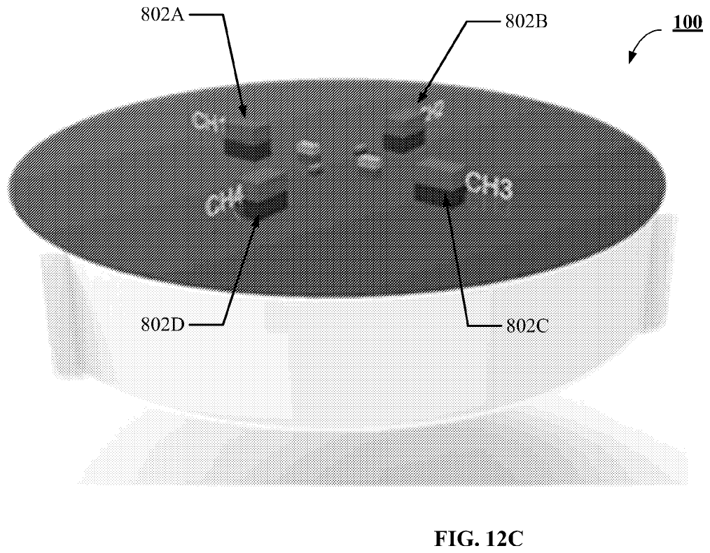

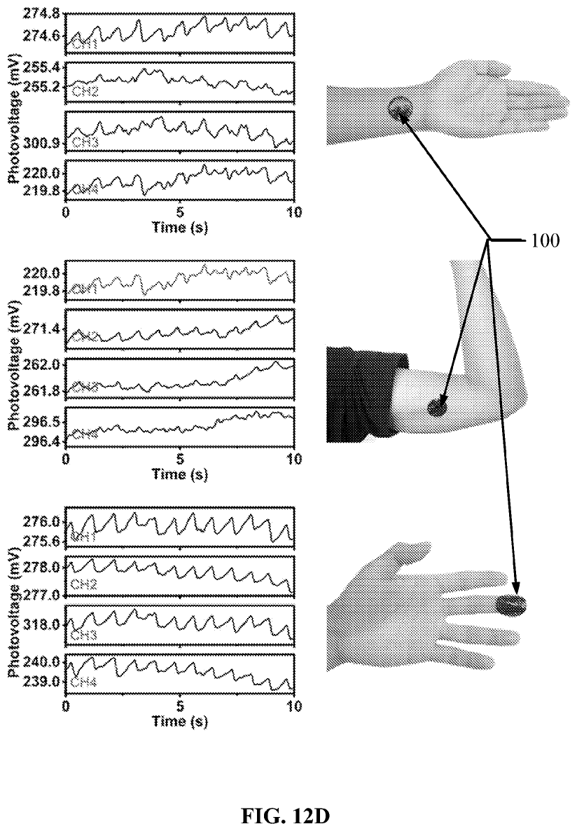

Different wearable uses of the sensing apparatus 100 are additionally demonstrated and tested, as well as effectiveness in optical/light-based oximetry applications. The thin design of the sensing apparatus 100 with flexible mechanics allows the sensing apparatus 100 to be worn on various body locations, including wrists, arms, legs, and/or the like. As shown in FIG. 12A, applying a tourniquet enables reduction of blood supply from the cardiovascular system to the end of the upper limb to test oximetry sensing in a limb hypoxia stage. The sensing apparatus 100 placed onto the corresponding forearm provides continuous monitoring of SpO₂, StO₂, pulsation intensity, respiration intensity, and/or other physiological measurements described herein, shown in FIG. 12B. With sensing modules arranged across a face of the sensing apparatus 100, as shown in FIG. 12C for example, the physiological measurements are measured at 4 different skin locations. FIG. 12B accordingly shows four signals for each of SpO₂, StO₂, PI, and RI physiological measurements. In various embodiments, multiple signals for a physiological measurement can be averaged together. As shown, application of tourniquet significantly weakens pulsation intensity at all four interface locations and tissue oxygenation at three out of the four interface locations, consistent with the resultant nature of tourniquet where the lack of local blood flow emerges. Transforming the pulsation patterns from real space into reciprocal space allows an enhanced revelation of signal correlation in response to tourniquet events.

Physical workout, including aerobic endurance training and anaerobic resistance training, also creates acute changes to oxygen saturation, heart rate, pulse intensity, and respiration intensity that can be captured with the wearable sensing patch. FIGS. 12A and 12B demonstrating the sensing stability of the device during certain physical motion and mechanical deformation. After a 1-min intense pedaling, the sensing apparatus 100 captured a significant increase in HR while the SpO₂ remains at ˜98% and StO₂ remains at ˜75%, indicating the state of aerobic training maintained by the user. The reciprocal analysis of the raw sensing signals suggests minimal interference from the motion artifacts and an enhanced intensity of pulsation patterns. FIG. 12D provides further experimental data related to positioning and placement of the sensing apparatus 100 relative to the subject's body. Specifically, placements of the sensing apparatus 100 on the wrist, bicep, and index finger are shown, along with resulting data.

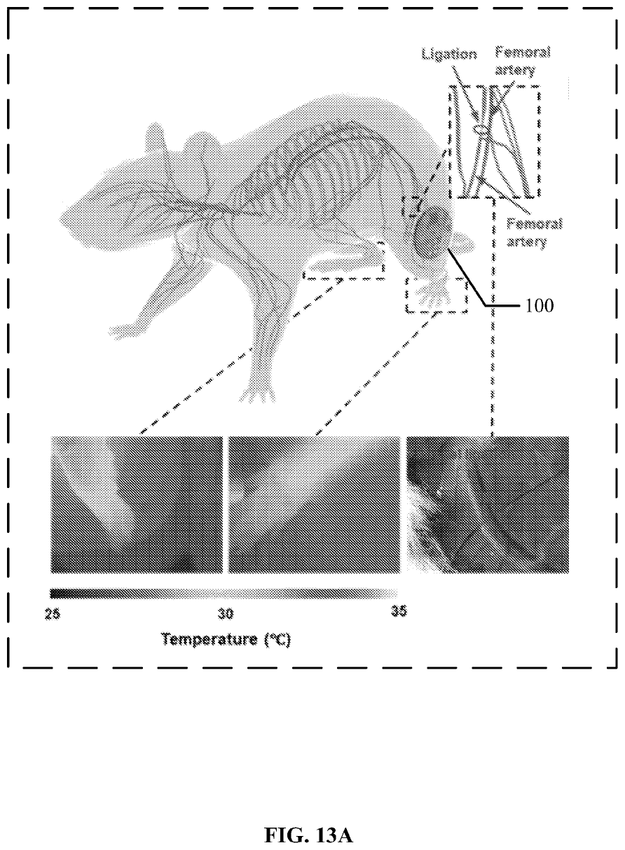

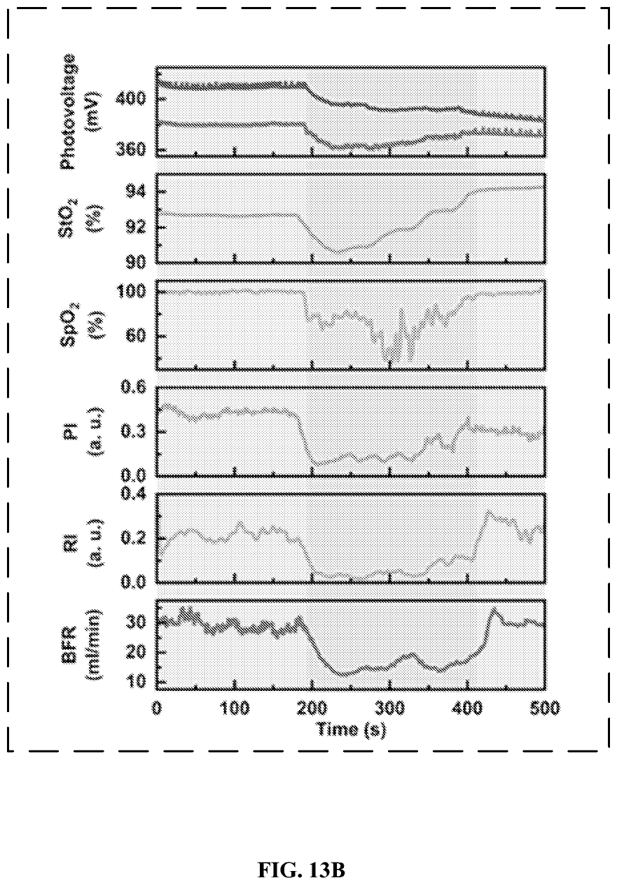

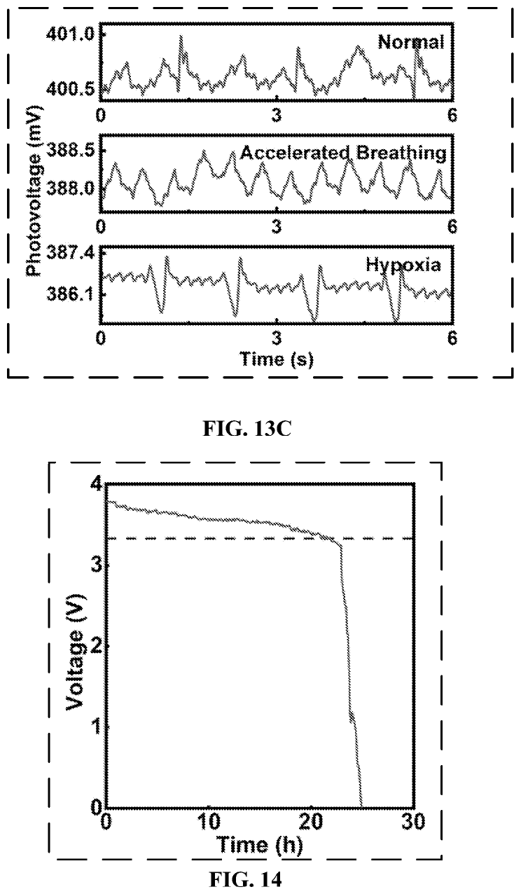

In addition to experimental configuration for subject use, the operation of the sensing apparatus 100 was further demonstrated in a rat model of hindlimb ischemia. Limb ischemia, a blockage in the arteries of the lower extremities, represents a typical form of PAD that requires immediate treatment to re-establish blood-flow to the affected area. Medical therapies including surgical or endovascular interventions are useful for symptomatic relief, but long-term results need a closely-monitoring strategy to enable timely actions. FIG. 13A relies on a model of murine hindlimb ischemia to demonstrate the continuous, multi-modality sensing capability of the wearable patch in capturing adverse events associated with limb ischemia. Here, a vessel ligation at the femoral artery is created by a strand of silk suture. FIG. 13A highlights the location of the ligation in the rat model as well as the deployment position of the wearable patch (with a 3-mm length microneedle array). Introduction of the ligation significantly reduces blood supply at the operated limb below the ligation rapidly, whereas the blood flow at the other limb without ligation remains normal, which is confirmed by comparing the respective inset thermal images. FIG. 13A includes an inset providing an enlarged view of the ligation on a limb artery.

FIG. 13B summarizes the multi-modal sensing information in an event of ligation and the following recovery, which includes the photovoltage from one of the 4 channels, the StO2, SpO2, the intensity of pulse and respiration, and the blood flow rate (represented by phase shift). As shown in FIG. 13B, the tightening of the suture stranded around the artery happens at time 180 s, creating a ligation event. At time 420 s, the release of the suture stand starts, which leads to the tissue recovery from the ligation. During the ligation and recovery period (as highlighted with a red background in FIG. 13B), StO2 and SpO2 physiological measures generated according to various example operations herein using the sensing apparatus 100 are remarkably lower than the normal status, and they both recover after removal of the ligation. The intensity of pulse and respiration are found to vanish during the ischemia stage resulting from ligation, and then recover to the normal value after removal of the ligation. The blood flow rate (BFR), which is characterized by the phase shift between a pair of waveform detectors 804 at the ligation spot, shows that the bloodstream flows slower than without ligation in the concerned stage.

Another test shown in FIG. 13C showed that the sensing apparatus 100 capturing the pulsation caused by respiration was sensitive to the change of the oxygen volume provided by the respirator where the frequency of the pulsation changes with the amount of oxygen supply. Specifically, higher oxygen supply (e.g., 2.6 ml/cycle) leads to higher respiration rate and vice versa. Therefore, the animal study demonstrates the multi-modal sensing information collected from the wearable patch can effectively detect warning signs related to peripheral arterial disease.

### 5. Power Source Experimentation

To test the lifetime of the electronic components (e.g., the control module 104, the sensing modules 106, a power source) of the sensing apparatus 100, a microcontroller was used to continuously measure battery voltage with respect to time. Specifically, a voltage divider is set up between the battery and the ADC inputs to the microcontroller, and then values from the original voltage of the battery (using a multimeter) are measured and correlated to the corresponding values read from the ADC. A regression analysis yielded the linear correlation between the battery and ADC values. Finally, the circuit was powered with a 3.7 V, 150 mAh battery, and the voltage was measured every 10 minutes from the ADC channel. The resulting data is plotted in FIG. 14. Battery life was defined as the length of time required to reduce the voltage to 90% of the baseline (3.7 V), which as shown is approximately at 22 hours.

Various embodiments described herein of integrating microneedle waveguides with a wireless optoelectronic system in a thin, flexible construction, provide sensing apparatuses capable of continuous, stable monitoring of oxygenation and other vital signs correlated to local tissues at depth, previously attainable only with implantable technology. The multi-channel sensing sites for spatially resolved monitoring and the wireless communication platform with interchangeable battery safely positioned outside the body highlight the advantageous outcome of such integration strategy compared with implantable sensors where strong requirements of materials for tissue environments may limit the achievable capabilities. Furthermore, the implementation of data analysis algorithms coupled with a neural network model enables high-quality, accurate detection of targeted physiological parameters. In vivo experiments with animal models of hind ischemia to track tissue oximetry, pulse oximetry, pulsation intensity, respiration intensity, and blood flow rate demonstrate the potential capability in monitoring warning signs associated with peripheral artery diseases. Thus, various embodiments enable robust deep tissue sensing and stimulation in a wearable and digital fashion, and across a broad range of clinical needs, including biochemical sensing, drug delivery, power supplies, thermal actuators, and/or the like.

### 6. Example Transdermal Drug Delivery

Transdermal drug delivery is of vital importance to therapeutic treatments. Skin provides convenient access for delivering most biotherapeutics and vaccines, typically via a hypodermic needle. However, patient compliance and/or adherence to a pharmaceutical plan of repetitive treatments over a long term remains to be a grand challenge. Furthermore, dynamic (and often unpredictable) trajectories of disease progression demands a pharmaceutical treatment that can be actively controlled in real time to ensure medical precision and personalization.

Hypodermic injection provides a low-cost and rapid way for drug delivery, but disposal safety, potential spread of bloodborne pathogens, and requirement of trained personnel impede its compliant implementation. Some strategies for transdermal drug delivery exploit mechanisms based on sonophoresis, iontophoresis, electroporation, photomechanical waves, heat, microneedles, and others. These strategies offer enhanced drug permeation through skin, safe and painless operation, easy-to-use procedures. However, lacking an automated mechanism to actively and precisely control and coordinate drug administration over a long period of time hinders their broad applicability for chronic pharmaceutical management. This becomes significantly critical for chronic diseases, including diabetes, hyperlipidemia, asthma, depression, and others, where repetitive drug administrations are required, and a dynamically personalized schedule of delivery could effectively improve drug efficacy and decrease drug toxicity. However, most drug delivery devices have limited capability in automating delivery digitally, especially outside of hospital settings and in a comfortable, long-lasting fashion.

Microneedles have shown great promise in facilitating delivery of various types of drugs, including small molecules, peptides, nucleic acids, and nano composites. Furthermore, modulating the structural integration and chemical functionalization of microneedles enables a broad range of release profiles. For example, microneedles with a core-shell structure that hosts a drug reservoir inside each needle can exhibit a pre-programmed, multi-step release profile with tunability via designing the degradation time of microneedle shell layers. This method also allows for the integration of multiple drugs to enable sequential release as a combined therapy. However, the complexity in fabrication also limits its manufacturing scalability. And the release time is pre-programmed and challenging to modify once the microneedles are deployed.

Chemical functionalization on microneedles provides a solution to introduce self-sensing and self-responsiveness features into microneedles. For insulin delivery, the material of microneedles can be functionalized with certain groups including phenylboronic acid or aminoimidazole, which react with glucose in body biofluids and induce structural changes in the polymer network of microneedles, thus triggering a release of embedded drug in correspondence to glucose levels in neighboring environments. This strategy allows for a self-regulated, long-term drug release to control chronic diseases conveniently, but the synthesis complexity and strong reliance on local microenvironments (instead of global body physiology) preclude its practical usage.

Apart from passive drug release, active control via external triggers to deliver drugs on demand offers enormous potential to form a closed-loop therapeutic system by integrating with associated health monitors that enhance treatment precision and dynamics. Heat-triggered release represents an example where microneedles with drug loaded inside a thermally responsive material (e.g. Expancel Microspheres and paraffin C₂₃) exhibit significant expansion in pore or volumetric size upon heating at, for example 47° C., to secrete the drug accordingly. Such mechanism allows drug release to be controlled with thermal triggers via an electrical heater or optical illumination. However, the inability to effectively focus thermal energy in a confined space of biological tissue precludes its delivery precision and safety.

Thin membranes based on biocompatible metals serving as a gate for drug reservoirs can enable actively controlled drug delivery. For example, the metallic gates can disintegrate or dissolve upon an electrical trigger. Some devices exploit various biocompatible metals including, but not limited to, Mg (for example, appropriately 30 μm in width), Mo (for example, appropriately 10 μm in width), and Au (for example, appropriately 300 nm μm in width) as metal gates to form electronic implants that enable on-demand drug delivery. Opening of the metallic gates by either anodic oxidation (e.g., for Mg and Mo) or corrosion-induced crevices (e.g., for Au) via electrical triggers can effectively initiate the active release behavior. The compatible integration via microfabrication technologies enables such a drug-releasing mechanism with high spatiotemporal controllability via small electrical signals (for example, +1.04 V vs SCE), which could potentially realize pharmaceutical automation both in space and time.

For example, various embodiments of the present disclosure provide for a spatiotemporal on-demand patch (SOP) that integrates drug-loaded microneedles with biocompatible metallic membranes to enable electrically triggered active control of drug release with high precision in space and time. In some embodiments, high-precision control (less than 1 mm²) of drug release is provided to targeted locations. In some embodiments, rapid release of drug (within 30 seconds) in response to electrical triggers is provided, and a multi-modal operation involving both drug release and electrical pulse stimulation from the same microneedle interface is provided for a SOP. In some embodiments, fabrication based on a solution molding strategy allows for high customizability and scalability to tailor the SOP for a broad range of pharmaceutical needs. In some embodiments, a wirelessly powered, digitally controlled SOP demonstrates promising potential in fully digital automation of drug delivery that enhances user adherence and ensures medical precision.

Embodiments herein provide a skin-interfaced drug delivery system that utilizes electrically triggered gated microneedles to realize on-demand drug delivery with high spatiotemporal controllability. In some embodiments, the drug delivery system (also referred to as spatiotemporal on-demand patch (SOP)) uses a thin Au layer (for example, approximately 150 nm in width) coated onto microneedles to enable drug encapsulation and protection at the standby stage. In some embodiments, small electrical triggers (for example, approximately 2.5 V direct current) for 30 seconds effectively disintegrate the Au coating, which exposes drug to initiate delivery. In one or more embodiments, microfabrication processes enable circuitry designs of the Au layer to realize release triggering of individual microneedles or subsections of them through a wireless communication module (such as, but not limited to, near-field communication, Bluetooth Low Energy (BLE)). In one or more embodiments, direct deposition of the Au layer onto microneedles overcomes limitations in the fabrication complexity and device robustness associated with the reservoir systems with free-standing metallic gates (such as those in implantable devices). In one or more embodiments, ultrafine spatial control (for example, less than 1 mm²) of drug release in a single microneedle, active management with high temporal (for example, less than 30 second) resolution of drug release, wireless operation, and comfort wearability are among the enabling capabilities of the SOP. In some embodiments, benchtop experiments using a fluorescent dye and in vivo study through intracranial injection demonstrate the use of SOP as a general, fully wireless, wearable platform for personalized, chronic drug delivery to improve pharmaceutical efficacy and user adherence.

As described above, various embodiments of the present disclosure provide an on-demand drug-delivery patch that can be digitally controlled to enable delivery precision in both space and time. In one or more embodiments, the spatiotemporal on-demand patch (SOP) uses bioresorbable microneedles with high-aspect ratios (for example, 3 to 8) as the drug-loading vehicle and a thin layer of Au (for example, having a thickness of 150 nm) as a release gate that can be digitally controlled with a small electrical trigger (for example, a direct current of 2.5 V for 30 seconds). In some embodiments, such design allows fully active control of drug release at a single-microneedle level with a spatial resolution of less than 1 mm², which highlights enabling capabilities of the present disclosure over other drug-delivery devices. In some embodiments, such spatial resolution allows more than twenty doses of drug to be housed within a thin wearable patch of 1 cm² in size, to ensure comfortable and convenient user adherence for repetitive pharmaceutical treatment over a long period of time. In some embodiments, the fabrication is compatible with microfabrication process, which could further decrease the spatial resolution to a micrometer level. In some embodiments, the on-demand, rapid-response drug release can be enabled within a 30-second post to an active electrical trigger.

In some embodiments, the fabrication procedure of an example SOP uses a simple solution-molding method with low cost and high dimensional customizability. In some embodiments, microneedles can be manufactured efficiently using laser ablation so that the microneedles may range from 0.6 mm to 3 mm and have high quality that fit a wide range of drugs (such as, but not limited to, melatonin with controllable payload). In one or more embodiments, the solution-based mold process allows drug-load PLGA microneedles to be produced at scale with high quality. In one or more embodiments, example processes of the present disclosure provide convenient compatibility for the following integration with electronic modules to enable digital automation in drug delivery.

In one or more embodiments, the SOP can feature wireless operation via near-field communication or Bluetooth Low Energy. In one or more embodiments, agar-soaking tests, fracture tests, and in vivo experiments for intracranial delivery validate the practical functionality and biocompatibility of the SOP. In one or more embodiments, the Au-coated microneedles can bring more functionality beyond drug-releasing control, as the SOP can offer in vivo electrical stimulation. In one or more embodiments, the multifunctionality of both drug delivery and stimulation therapy could offer a synergistic effect in creating advanced therapy, as potential future work. In one or more embodiments, these concepts establish unique approaches in high-precision drug-delivery technologies with additional utilities in advancing fundamental studies of disease pathology (such as cancer metastasis) and neuroscience research. In one or more embodiments, future efforts on fully digital automation of drug delivery will pave the way for the next generation of precision medicine.

For example, one or more embodiments provide a microneedle and an electrically triggerable membrane encapsulating the microneedle and defining at least one reservoir between the microneedle and the electrically triggerable membrane. In one or more embodiments, the electrically triggerable membrane comprises electrically triggerable gold. In one or more embodiments, the membrane width associated with the electrically triggerable membrane is between 145 nanometers and 155 nanometers. One or more embodiments provide a controller coupled to the microneedle and configured to transmit an electrical trigger to the microneedle to cause a disintegration of the electrically triggerable membrane and a release of content from the at least one reservoir. In one or more embodiments, the electrical trigger comprises a direct current signal between 2 volts and 3 volts. In one or more embodiments, the controller is configured to receive a release control signal and, in response to the release control signal, transmit the electrical trigger to the microneedle. In one or more embodiments, the controller comprises at least one of a near-field communication module or a Bluetooth module. In one or more embodiments, the release control signal comprises a microneedle indication associated with the microneedle. One or more embodiments provide for a microneedle array comprising a plurality of microneedles that includes the microneedle. In one or more embodiments, the electrically triggerable membrane encapsulates each of the plurality of microneedles. In one or more embodiments, the controller is configured to receive a plurality of release control signals. In one or more embodiments, the controller is configured to determine one or more microneedles from the plurality of microneedles that are associated with the plurality of release control signals. In one or more embodiments, the controller is configured to transmit one or more electrical triggers to the one or more microneedles.

One or more embodiments provide for a sensing apparatus for deep tissue sensing and transdermal delivery. In one or more embodiments, the sensing apparatus comprises a base layer configured to interface with a skin surface of a subject, a sensing layer positioned above the base layer and comprising one or more waveform detectors and one or more waveform generators configured to emit wave signals, a microneedle attached to a skin-interfacing portion of the base layer and configured to waveguide the wave signals into a deep tissue of the subject, and an electrically triggerable membrane encapsulating the microneedle and defining at least one reservoir between the microneedle and the electrically triggerable membrane. One or more embodiments further provide for a controller coupled to the microneedle and configured to transmit an electrical trigger to the microneedle to cause a disintegration of the electrically triggerable membrane and a release of content from the at least one reservoir. In one or more embodiments, the controller is configured to receive a release control signal and, in response to the release control signal, transmit the electrical trigger to the microneedle. In one or more embodiments, the release control signal comprises a microneedle indication associated with the microneedle.

One or more embodiments further provide a microneedle array comprising a plurality of microneedles that includes the microneedle. In one or more embodiments, the electrically triggerable membrane encapsulates each of the plurality of microneedles. In one or more embodiments, the one or more waveform generators comprise one or more light-emitting diodes configured to emit light signals. In one or more embodiments, the microneedle is configured as an optical waveguide for the light signals. In one or more embodiments, the light signals include visible red light signals and near-infrared signals. In one or more embodiments, the one or more waveform generators comprise one or more ultrasonic generators configured to emit ultrasonic signals. In one or more embodiments, the microneedle is configured to act as ultrasonic waveguides for the ultrasonic signals.

One or more embodiments further provide for a controller in electronic communication with the one or more waveform generators and the one or more waveform detectors. In one or more embodiments, the controller is configured to operate the one or more waveform generators to define a sensing field within the deep tissue of the subject via the wave signals and the microneedle as a waveguide and generate sensing data based at least in part on reflected wave signals detected at the one or more waveform detectors. In one or more embodiments, the controller is further configured to transmit, via wireless communication, the sensing data to a workstation.

### 7. Example Spatiotemporal On-Demand Path (SOP) Configurations

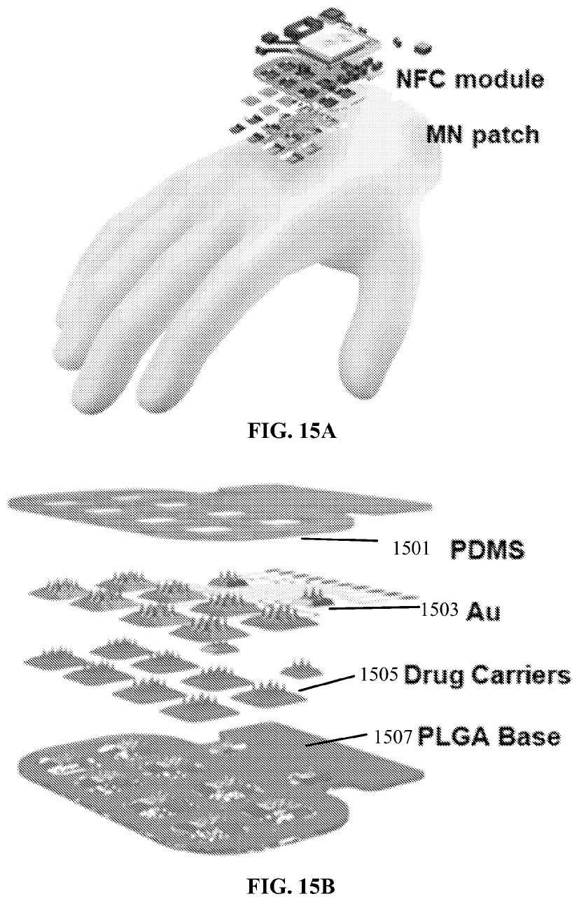

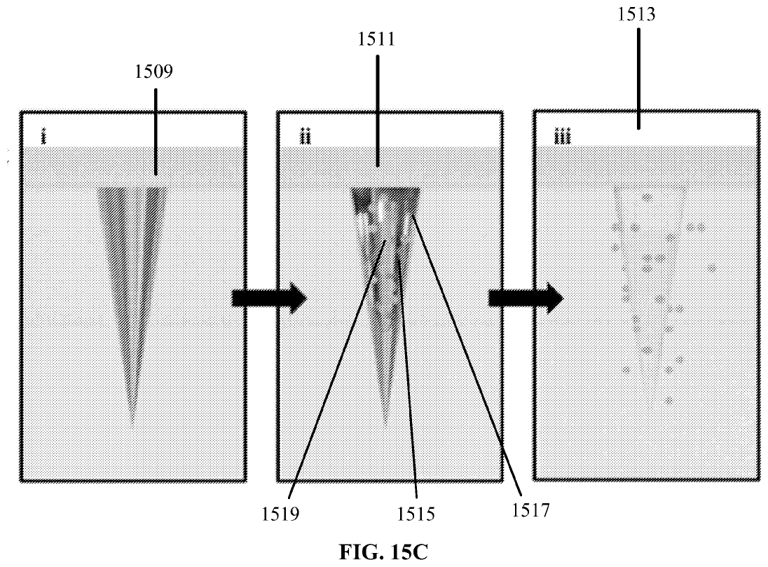

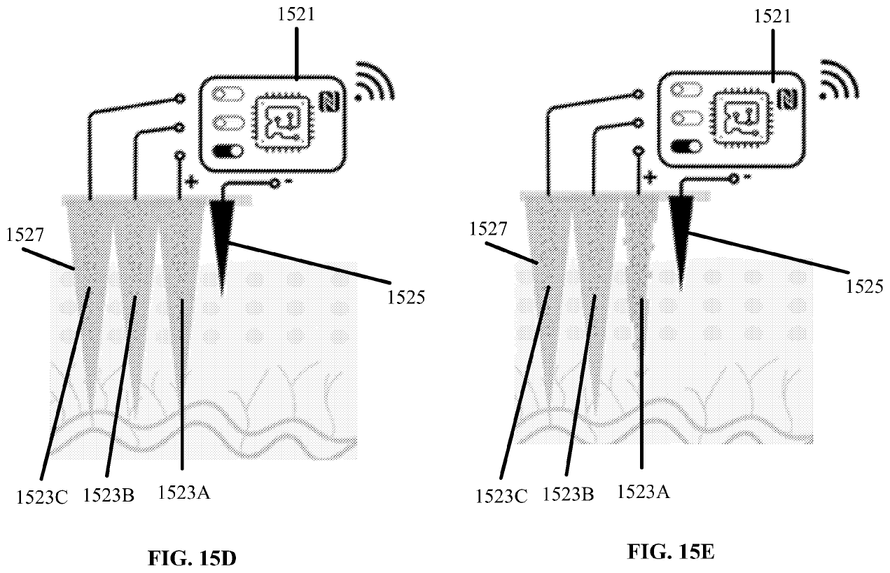

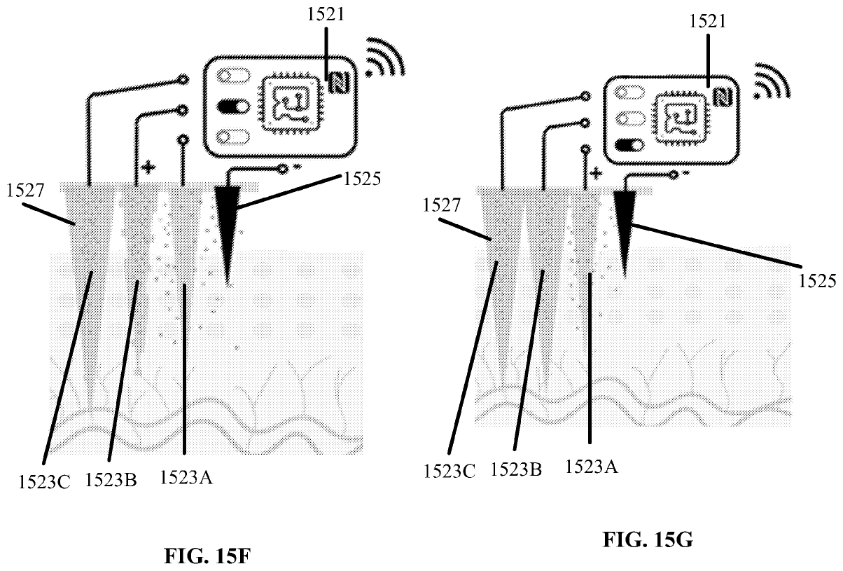

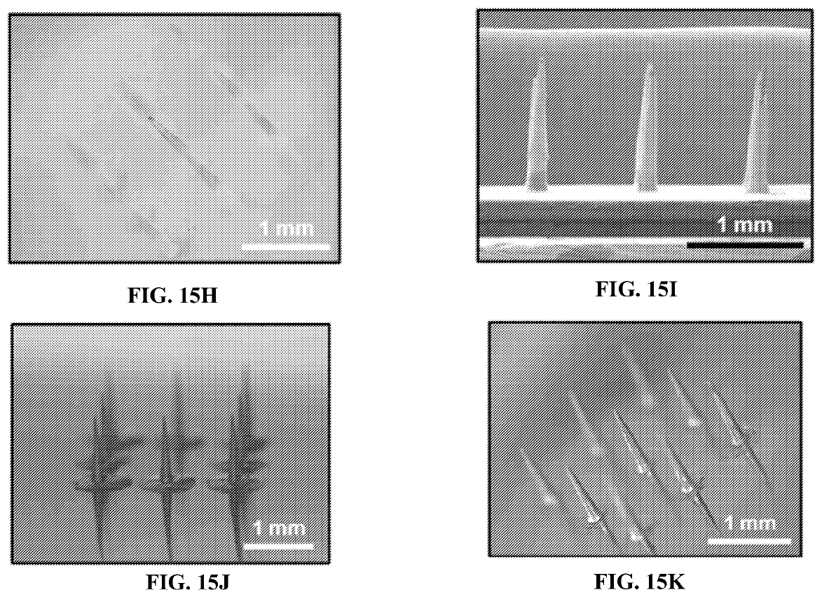

Turning now to FIG. 15A, FIG. 15B, FIG. 15C, FIG. 15D, FIG. 15E, FIG. 15F, FIG. 15G, FIG. 15H, FIG. 15I, FIG. 15J, and FIG. 15K, example spatiotemporal on-demand patches for wireless, active control of drug delivery in accordance with example embodiments of the present disclosure are illustrated.

FIG. 15A depicts a schematic illustration highlighting the construction of a wirelessly controlled spatiotemporal on-demand patch (SOP) for high-precision drug delivery in accordance with an example embodiment.

In one or more embodiments, the SOP features components such as, but not limited to, an array of drug-loaded microneedles protected by active encapsulation that exploits electrochemically triggered crevice corrosion for on-demand drug delivery, as well as a NFC module assembled on a soft printed-circuit board for wireless control.

In some embodiments, the drug-loaded microneedles are protected with an electrochemically triggerable metal layer as the drug delivery interface. In some embodiments, the NFC module provides wireless control of triggering signals in controlling the location and schedule of drug release.

In one or more embodiments, a flexible printed circuit board (PCB) provides interconnected traces that integrate the two parts together to form a fully wireless, wearable drug delivery system. In one or more embodiments, the main body of the drug delivery interface (including the microneedle arrays) uses poly(D,L-lactide-co-glycolide) (PLGA) as the matrix material that can undergo bulk erosion upon contact with biofluids to generate biological benign byproducts (lactic acid and glycolic acid).

FIG. 15B depicts an exploded view of an example apparatus for transdermal delivery. In particular, the example apparatus shown in FIG. 15B provides an example drug-delivery interface of an example SOP.

In accordance with various examples of the present disclosure, the example apparatus for transdermal delivery comprises a microneedle and an electrically triggerable membrane. In the example shown in FIG. 15B, the example apparatus includes a PDMS encapsulation 1501, a gold (Au) coating 1503, drug-loaded microneedles 1505 based on poly(D,L-lactide-co-glycolide) (PLGA), and a PLGA substrate 1507. In such an example, the electrically triggerable membrane of the example apparatus for transdermal delivery comprises the gold coating 1503, and the microneedle of the example apparatus for transdermal delivery comprises the drug-loaded microneedles 1505.

As shown in FIG. 15B, an example fabrication relies on sputter deposition to coat a thin layer of Au (thickness 150 nm) onto the drug-loaded microneedle arrays supported by a PLGA base. In one or more embodiments, ablation using a laser beam defines the Au traces that connect with separated microneedle domains to realize spatial control of drug release. In one or more embodiments, a thin layer of polydimethylsiloxane (PDMS) (for example, with a thickness of 10 μm) covers the Au layer at the regions of circuitry base and exposes only the microneedle regions to allow physical contact of the microneedle Au with biofluids.

As shown in FIG. 15B, the drug-loaded microneedles 1505 is also referred to as a drug carriers. In particular, the electrically triggerable membrane (for example, the gold coating 1503) encapsulates the microneedle (for example, the drug-loaded microneedles 1505) and defines at least one reservoir between the microneedle and the electrically triggerable membrane. In some embodiments, the at least one reservoir may provide housing for drugs for transdermal delivery.

In the present disclosure, a material is “electrically triggerable” when the material changes its shape, size, or structure, and/or other physical characteristics (such as, but not limited to, disintegrates, corrodes, bends, twists, deforms, distorts, and/or the like) in response to electrical triggers (for example, but not limited to, a direct current signal). For example, an example electrically triggerable membrane refers to a membrane that may corrode and/or disintegrate in response to an electrical trigger (such as, but not limited to, a direct current). As another example, an example electrically triggerable gold refers to gold material that may corrode and/or disintegrate in response to an electrical trigger (such as, but not limited to, a direct current). Additional example details associated with electrically triggerable membranes and electrically triggerable gold are described herein.

FIG. 15C depicts an example schematic illustration showing an example process of electrically controlled on-demand drug delivery from an individual microneedle in accordance with an example embodiment. In the example shown in FIG. 15C, an electrically triggerable membrane defines an an encapsulation layer. Example stages include a standby stage 1509 (where an encapsulation layer protects the microneedle from releasing drug), a transitioning stage 1511 (where an electrical trigger initiates crevice corrosion of the encapsulation layer to expose drug-loaded base), and a releasing stage 1513 (where exposed base starts to release drugs).

For example, drugs are loaded in the at least one reservoir 1515 between the microneedle 1519 and the electrically triggerable membrane 1517. Similar to those described above in connection with at least FIG. 15B, the electrically triggerable membrane 1517 may comprise electrically triggerable gold. In some embodiments, a membrane width associated with the electrically triggerable membrane 1517 is between 145 nanometers and 155 nanometers, providing technical benefits and advantages such as, but not limited to, easy releasing of the drugs from the at least one reservoir 1515.

Referring now to FIG. 15D to FIG. 15G, example schematic illustrations demonstrate the capability of spatiotemporal control of releasing profile from the SOP in accordance with an example embodiment. In the examples shown in FIG. 15D to FIG. 15G, the SOP is deployed the skin interface in accordance with an example embodiment.

In some embodiments, the SOP comprises a controller 1521 that is coupled to and communicates with microneedles (for example, the microneedle 1523A, the microneedle 1523B, and the microneedle 1523C) to enable active control of drug release for each individual microneedle (for example, the microneedle 1523A, the microneedle 15238, and the microneedle 1523C).

In some embodiments, a controller 1521 may comprise one or more processing elements, similar to the various examples described above.

In some embodiments, the controller 1521 may comprise one or more communication modules (such as, but not limited to, a near-field communication module or a Bluetooth module).

As described above, the controller 1521 is coupled to one or more microneedles (for example, the microneedle 1523A, the microneedle 1523B, and the microneedle 1523C). In some embodiments, the controller 1521 is configured to transmit an electrical trigger to the one or more microneedles to cause a disintegration of the electrically triggerable membrane 1527 that covers the one or more microneedles and a release of content from the at least one reservoir.

For example, the controller 1521 may receive a release control signal. In the present disclosure, the term “release control signal” refers to an electronic signal that indicates a request to initiate a release of content from the at least one reservoir. For example, the release control signal may be wirelessly transmitted from a client device (for example, a computer, a mobile smart phone, and/or the like) to the controller 1521 of the SOP. In some embodiments, the release control signal is non-transitory.

In some embodiments, the controller 1521 receive a release control signal, and, in response to the release control signal, transmit one or more electrical triggers to one or more microneedles (for example, the microneedle 1523A, the microneedle 1523B, and the microneedle 1523C).

In some embodiments, the electrical trigger comprises a direct current signal between 2 volts and 3 volts, which provide technical benefits and advantages such as, but not limited to, triggering the electrically triggerable membrane without causing harm to user wearing the SOP.

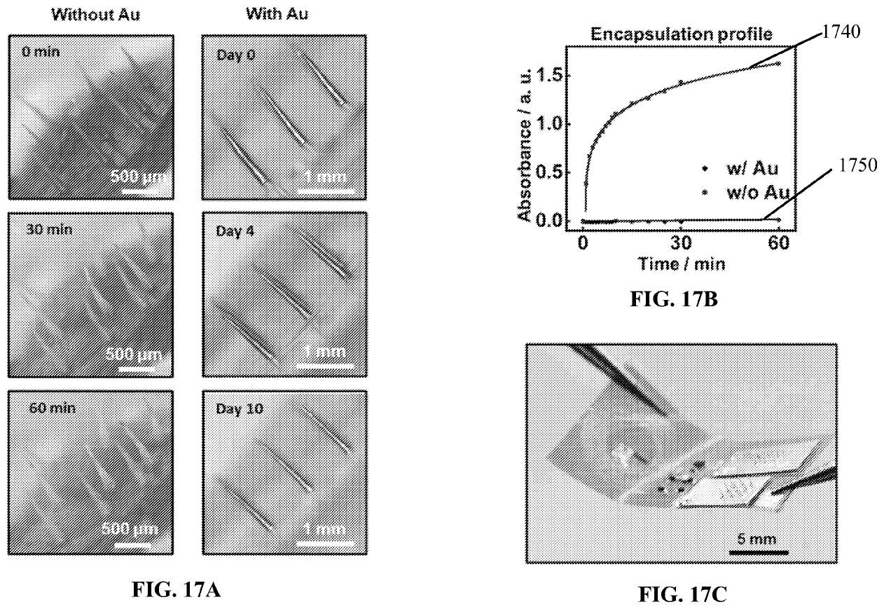

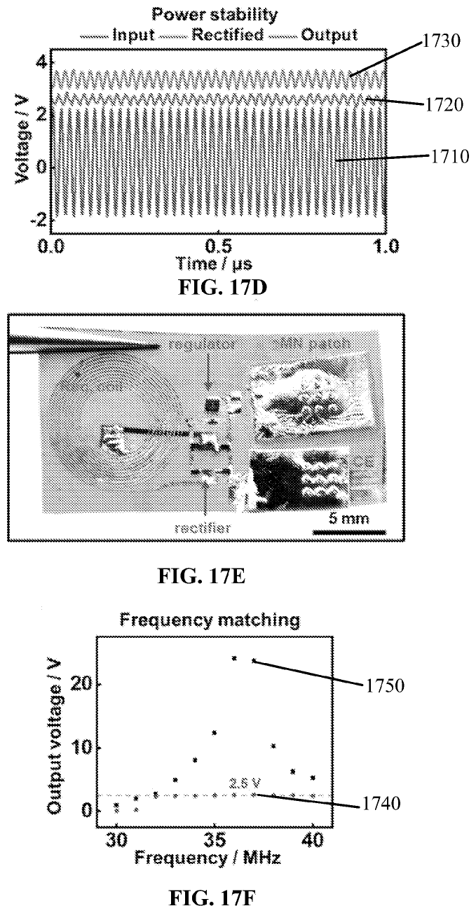

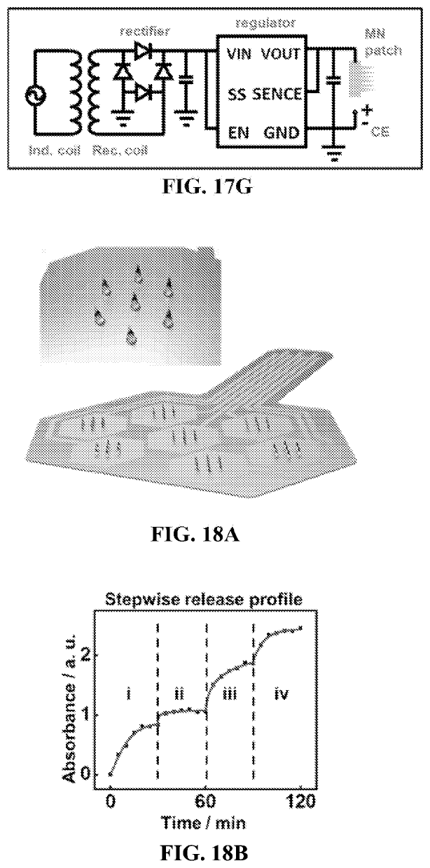

In some embodiments, the controller 1521 may generate electrical triggers based on implementing a signal generator, an inductive coil, and a receiver coil, details of which are described in connection with at least FIG. 17A to FIG. 17G. Additionally, or alternatively, the controller 1521 may control the operation of a switch that is connected between a power source (such as, but not limited to, a battery) and one or more microneedles. In such an example, the controller 1521 may cause the switch to be turned on when the controller 1521 receives the release control signal, thereby providing a direct current signal to the microneedle from the power source.

In the examples shown in FIG. 15D to FIG. 15G, a microneedle array is implemented. In particular, the microneedle array comprises a plurality of microneedles that includes the microneedle 1523A, the microneedle 1523B, and the microneedle 1523C. In the example shown in FIG. 15D to FIG. 15G, the electrically triggerable membrane 1527 encapsulates each of the plurality of microneedles (including the microneedle 1523A, the microneedle 1523B, and the microneedle 1523C).

FIG. 15D to FIG. 15G further illustrate examples of individual controls of releasing drugs from each individual microneedles in the microneedle array. For example, the release control signal may comprise one or more microneedle indications associated with one or more microneedles. In the present disclosure, the term “microneedle indication” refers to data from a release control signal that uniquely identifies one or more microneedles from the microneedle array.

For example, the microneedle indication from the release control signal may uniquely identifies the microneedle 1523A from the microneedle array, as shown in FIG. 15D to FIG. 15E. In such an example, the controller 1521 transmits an electrical trigger (for example, but not limited to, a direct current) to the microneedle 1523A from the microneedle array. As shown in FIG. 15D and FIG. 15E, the microneedle array also comprises a microneedle 1525 (or an counter electrode) that is connected to the ground. As such, the direct current flows from the microneedle 1523A to the microneedle 1525, triggering portions of the electrically triggerable membrane 1527 that cover the microneedle 1523A to disintegrate and to release the drugs loaded on the microneedle 1523A.

Referring now to FIG. 15F, the microneedle indication from the release control signal may uniquely identifies the microneedle 1523B from the microneedle array. In such an example, the controller 1521 transmits an electrical trigger (for example, but not limited to, a direct current) to the microneedle 1523B from the microneedle array. As shown in FIG. 15F, the microneedle array also comprises a microneedle 1525 that is connected to the ground. As such, the direct current flows from the microneedle 1523B to the microneedle 1525, triggering portions of the electrically triggerable membrane 1527 that cover the the microneedle 1523B to disintegrate and to release drugs loaded on the microneedle 1523B.

As such, in accordance with various embodiments of the present disclosure, the controller 1521 may receive a plurality of release control signals and determine one or more microneedles from the plurality of microneedles that are associated with the plurality of release control signals. For example, the controller 1521 may determine that the plurality of release control signals provides microneedle indications associated with the microneedle 1523A and the microneedle 1523B, and transmit electrical triggers to the microneedle 1523A and the microneedle 1523B to cause releasing the drugs loaded on the microneedle 1523A and microneedle 1523B.

FIG. 15H depicts an optical image of a PLGA microneedle array in accordance with an example embodiment. FIG. 15I depicts a corresponding SEM image with a tilted view on the PLGA microneedle array in accordance with an example embodiment. FIG. 15J depicts an optical image of a PLGA microneedle array loaded with Rhodamine B in accordance with an example embodiment. FIG. 15K depicts an optical image of a PLGA microneedle array protected with an electrically triggerable encapsulation (for example, with an Au layer having a thickness of 150 nm) in accordance with an example embodiment. In some embodiments, the length and base diameter of the example microneedles in FIGS. 15H-15K are around 1.2 mm and 270 μm, respectively. In some embodiments, the length and/or the base diameters may be different from those values.

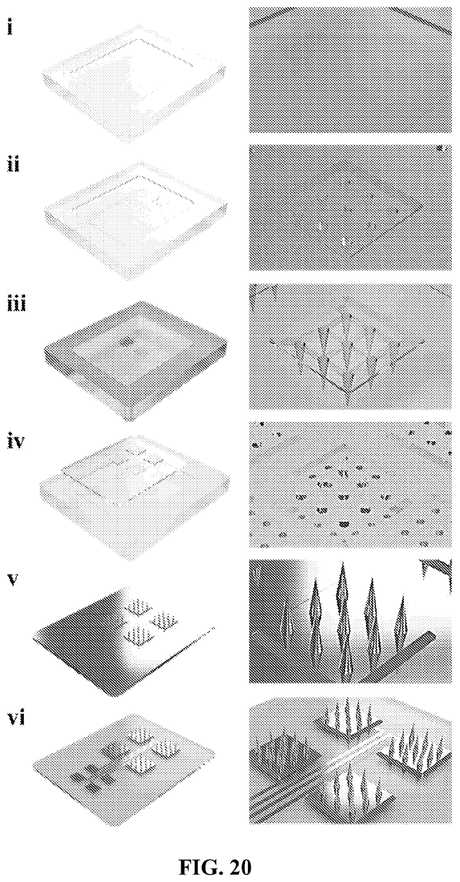

In one or more embodiments, an example fabrication method of the SOP may be based on a low-cost solution-molding procedure as illustrated in FIG. 20. In one or more embodiments, the example fabrication method starts with UV laser ablation to define a microneedle mold from a PDMS pad. In one or more embodiments, the example fabrication method includes drop-casting a drug loaded PLGA solution into the PDMS mold and setting under vacuum to allow the entrance of PLGA into the negative microneedle molds. In one or more embodiments, the example fabrication method includes sufficient solidification over 8 hours to allow easy extraction of the PLGA microneedles. In one or more embodiments, the example fabrication method includes the PLGA microneedles undergoing sputtering deposition to coat a 150 nm-thick Au layer that conformally covers the top surface, followed by patterning with a laser ablation system to define control circuits. In one or more embodiments, the example fabrication method includes drop casting a thin PDMS layer (for example, with a thickness of 10 μm) onto the Au layers to protect the control circuits and expose the microneedles for drug release. In one or more embodiments, the microneedle patch is then attached onto a flexible PCB and connected with a current regulator, a wireless energy harvester, and a microcontroller to complete the fabrication process.

In one or more embodiments, the solution-molding method produces SOPs with: i) tunable microneedle lengths from 600 μm to 3 mm and aspect ratio from 3 to 8; ii) high dimension uniformity in both length and base diameter as illustrated in FIG. 17A; and iii) arbitrary microneedle array configurations (square, hexagonal, single-needle, etc.).

FIGS. 15H-15K illustrate that, in one or more embodiments, the morphology and shape of the PLGA microneedles remain stable during both Au deposition and drug loading procedures.

FIG. 15C to FIG. 15E illustrate an example overall working mechanism to realize high-precision drug delivery of the SOP. In one or more embodiments, the electrically triggered crevice corrosion of the Au protective layer serves as the switch for initiating drug release of specific collections of SOP microneedles. In one or more embodiments, upon the SOP being deployed on the skin, the microneedles stay at a standby stage with the drug fully protected by the Au layer. In one or more embodiments, once a direct current electrical trigger (for example, between 2.2 V and 3 V) is applied, the electrochemical crevice corrosion starts to occur on the triggered microneedles, transitioning them from standby mode to releasing mode. In one or more embodiments, after a short period (e.g., less than 30 seconds when 2.5 V is applied), the Au protective layer on the microneedles is fully dissolved and microneedles are exposed to the bioenvironment to enable drug release. In one or more embodiments, the compatible integration of microcontrollers allows the electrical triggers to be manipulated at a precise timepoint for precision delivery. In one or more embodiments, by patterning Au layers via microfabrication, the SOP can realize spatial profile of drug release at a high spatial resolution (for example, approximately 1 mm²). In one or more embodiments, a built-in anode is integrated into the SOP to complete the circuit for the in vivo electrochemical crevice corrosion.

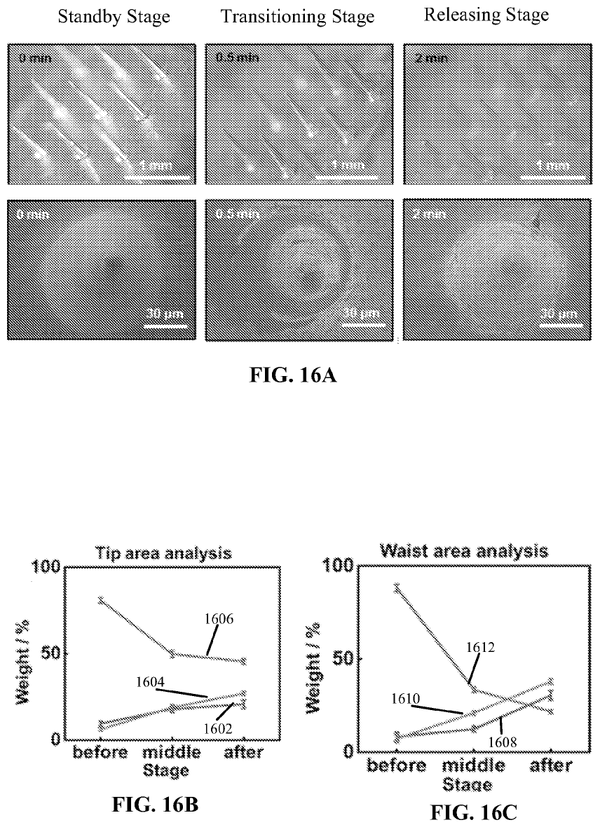

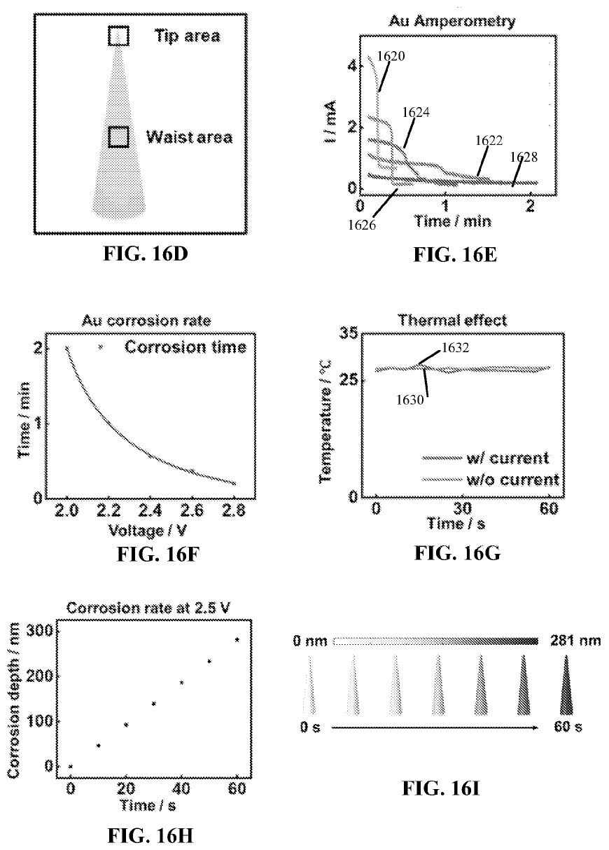

FIG. 16A to FIG. 16I illustrate electrically triggerable encapsulation for active control of drug release.

For example, FIG. 16A depicts optical images and the corresponding SEM to demonstrate a SOP undergoing the process of electrically controlled crevice corrosion. In one or more embodiments, initially (for example, at 0 minute), the drug-loaded microneedles are fully protected by a layer of gold (for example, with a thickness of 150 nm). In one or more embodiments, at 0.5 minutes, an electrical trigger (for example, 2.5 V and in 1× Dulbecco's phosphate-buffered saline) activates the crevice corrosion of the gold encapsulation layer. In one or more embodiments, the exposed drug embedded inside the microneedle core then (for example, at 2 minutes) starts to diffuse to the fluidic environment.

FIGS. 16B-16C depict element analyses by energy-dispersive X-ray spectroscopy (EDXS) of the three stages (as shown in FIG. 16A) of the on-demand releasing process of drug. FIG. 16B corresponds to tip areas of microneedle. In particular, line 1602 corresponds to the weight percentage of C, line 1604 corresponds to the weight percentage of O, and line 1606 corresponds to the weight percentage of Au. FIG. 16C corresponds to waist areas of microneedle. In particular, line 1608 corresponds to the weight percentage of C, line 1610 corresponds to the weight percentage of O, and line 1612 corresponds to the weight percentage of Au.

FIG. 16D provides a schematic illustration indicating the corresponding areas of a microneedle analyzed by EDXS element mapping. FIG. 16E depicts an amperometry characterization of the crevice corrosion process of the gold layer (for example, with a thickness of 150 nm) coated on microneedles with a height of 1.2 mm. In particular, line 1620 corresponds to 2.0 V, line 1622 corresponds to 2.2 V, line 1624 corresponds to 2.4 V, line 1626 corresponds to 2.6 V, and line 1628 corresponds to 2.8 V. FIG. 16F depicts a measured relationship between the corrosion time and the trigger potential applied on the gold layer (for example, with a thickness of 150 nm) based on the amperometry curve in FIG. 16E. FIG. 16G depicts an analysis of thermal effect during the crevice corrosion process, indicating negligible heat generated in the system. In particular, line 1630 corresponds to when the current is applied, and line 1631 corresponds to when the current is not applied.

FIG. 16H depicts the simulation of crevice corrosion depth on microneedle under 2.5-V in 1× Dulbecco's phosphate-buffered saline. FIG. 16I depicts the corresponding corrosion profile on a microneedle in 1 min (for example, 2.5 V and with 1× Dulbecco's phosphate-buffered saline) related to FIG. 16H. FIG. 16A to FIG. 16I demonstrate the characterization of SOP on its active control of drug release (for example, based on microneedles with a length of 1.2 mm and having a gold coating with a thickness of 150 nm as an electrically triggerable membrane). In one or more embodiments, the operation of drug-release control includes three stages: 1) a standby stage when the microneedles are fully coated with Au (labeled as 0 min); 2) a transitioning stage when the electrical trigger is triggered and the Au layer is partially dissolved (labeled as 0.5 min); and 3) a releasing stage when the Au is fully dissolved and microneedles are exposed to the biofluids (labeled as 2 min).

In one or more embodiments, 1× Dulbecco's phosphate buffered saline (for example, based on DPBS from Corning) is used to simulate the body biofluid.

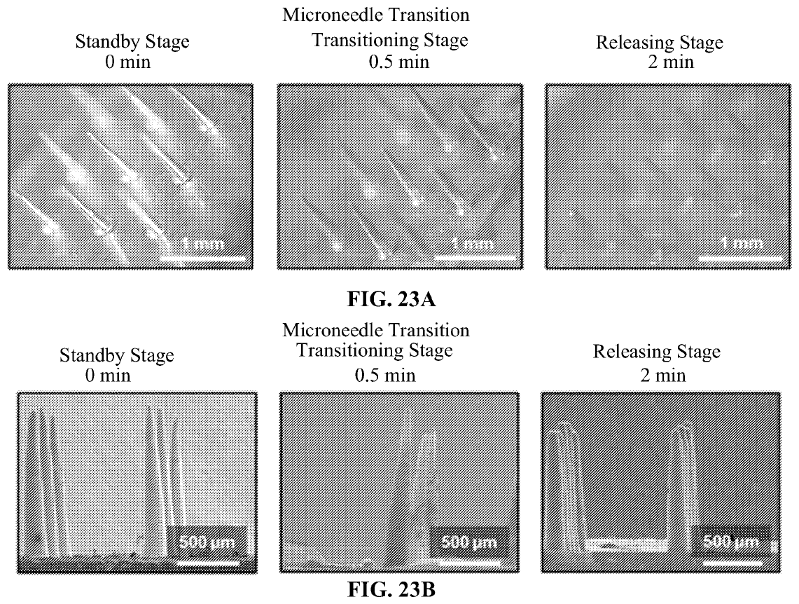

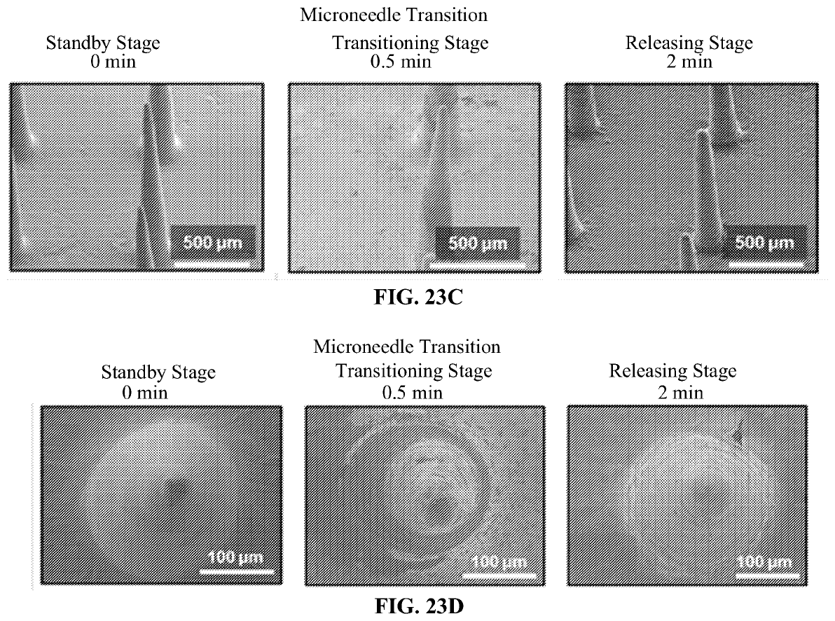

For example, an experiment is conducted at room temperature and triggered by a 2.5-V DC current. FIG. 16A shows optical and SEM images of the microneedle arrays at different stages, indicating an obvious change in surface color and roughness associated with the electrically triggered crevice corrosion. The results demonstrate that the main structure of the microneedle stays stable during the transitioning stage and the electrical triggers effectively dissolves Au into biofluids and sufficiently expose the core microneedles. More characterizations from different perspectives are illustrated in FIG. 23A to FIG. 23D.

In one or more embodiments, the electrochemical crevice corrosion of the Au layer can be triggered by a direct current (DC) potential within 2 minutes, which is of clinical relevance for a timely response in drug administrations. In one or more embodiments, a quantitative amperometry study of current density under different potentials is conducted to understand the electrical triggering behavior. FIG. 16E depicts I-V measurements of SOP triggering with potential bias ranging from 2.0 V to 2.8 V applied to the microneedle arrays (for example, with microneedles having 1.2 mm in length and a gold coating with a thickness of 150 nm as an electrically triggerable membrane). In one or more embodiments, a steep drop in current density appears at 15 seconds of application for 2.8 V bias indicating the endpoint of the electrochemical crevice corrosion. In one or more embodiments, the time of the current-drop appearance increases as the potential bias decreases.

In one or more embodiments, the time consumption from the beginning to the end of current is defined as the effective corrosion time, which is plotted against potential in FIG. 16F. In one or more embodiments, the fitting is based on Butler-Volmer equation (I=i_{corr}[exp(−σnFη/RT)−exp((1−σ)nFη/RT)], where i_{corr} is the exchange current density, F is the Faraday constant, η is the overpotential, R is the ideal gas constant, T is the thermodynamic temperature, a is a coefficient with values ranging from 0 to 1, and n is the number of electrons in the anodic half reaction. As shown, the fitting describes the relationship between potential difference and reaction rate.

In one or more embodiments, at an ambient potential of 2.5 V, the crevice corrosion on microneedle is triggered within 30 seconds. This parameter is applied in the following experiments that test the hypothesis where the crevice corrosion driven by constant voltage includes two parts, anodic oxidation and mechanical crevices.

In one or more embodiments, the 2.5 V potential between anode and cathode is sufficient for triggering gold oxidation coupled with hydrogen emission reaction (HER) at ambient conditions (for example, 1×DPBS at 25° C.). In one or more embodiments, an anodic potential larger than 1.95 V (as compared to the standard hydrogen electrode or SHE) is high enough to drive the below-surface oxidation of gold film. In one or more embodiments, the crevice corrosion is triggered at a potential above 2.0 V, even though the HER on the counter electrode is not under the standard condition. In one or more embodiments, the oxidated Au-species in neutral and alkaline environments may include oxides, hydroxides, and free elements, mostly in the form of nanoparticles (NPs), that collectively in the amount used here are benign to human body.

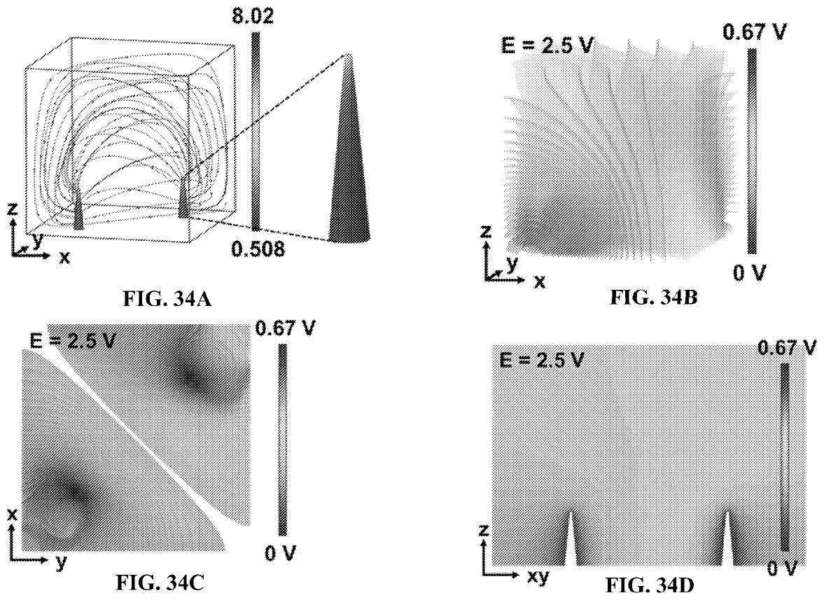

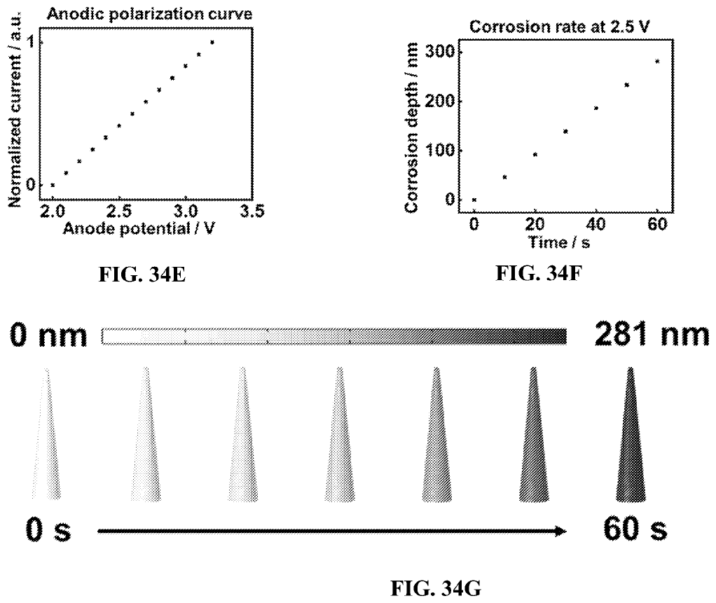

As depicted in FIG. 34A to FIG. 34D, the anodic behavior is also studied by multi-physics simulation including reaction potential, reaction rate, current and potential distribution. As shown in FIGS. 16H and 16I, the simulation results show the reaction rate at 2.5 V is 281 nm/min, which is consistent with the experimental observations (for example, 150 nm in approximately 30 seconds). As depicted in FIG. 34E, the anodic polarization curve under the condition of standard potentials and primary current distribution also show a minimal triggering potential at around 2.0 V. However, in one or more embodiments, the anodic oxidation may not account for the entire loss of Au coating. Based on the 2.6-V amperometry experiment depicted in FIG. 16E, the theoretical weight of the oxidated gold is calculated as 122 μg. In one or more embodiments, the actual value is lower because water splitting (for example, oxygen generation) as the side reaction may also contribute to the current density. In one or more embodiments, this amount of Au NPs does not constitute a health hazard since it is much lower than the safe exposure threshold of 5 mg/ml.

In one or more embodiments, the total weight of the diminished Au film on the microneedle array is calculated as 290 μg, which is significantly higher than the oxidated amount. In one or more embodiments, this is a result of a major part of Au layer not being oxidated but exfoliated from the surface due to crevices and cracks. In one or more embodiments, these defects come from a weakening effect on Au membrane as they become thinner during corrosion. In one or more embodiments, this kind of crevice on metal film is generated by an electrical current.

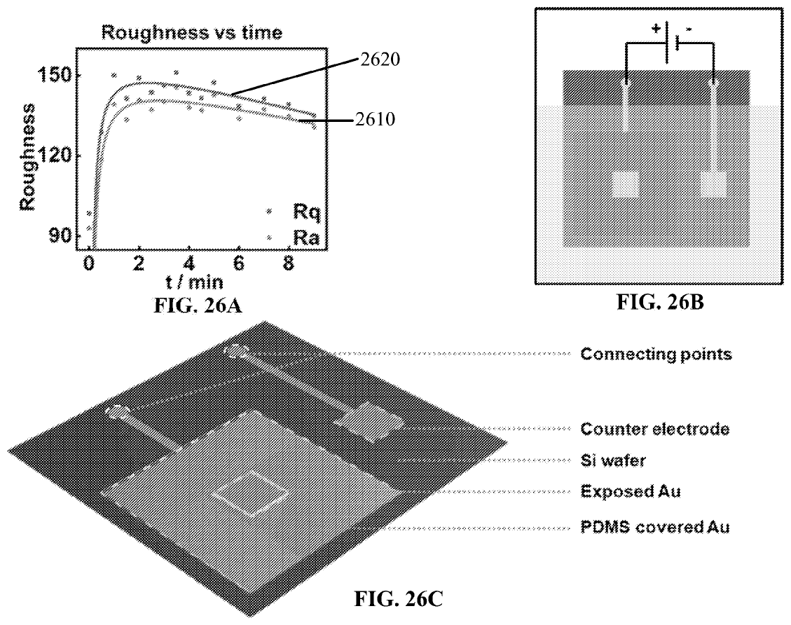

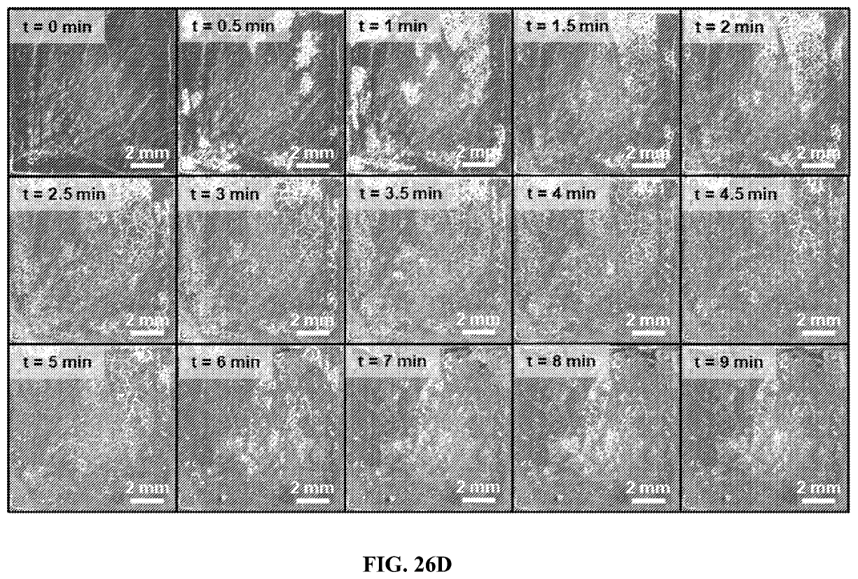

In one or more embodiments, the presence of mechanical crevices and exfoliations is validated by an Au-on-wafer experiment. The surface roughness of the crevice corrosion of gold layer is also studied with an Au-on-wafer (for example, a {100}facet, 150-nm, 1 cm² square) model as depicted in FIG. 26B to FIG. 26C. In one or more embodiments, the gold layer is connected to a power source and crevice corrosion is triggered at 2.5 V in 1×DPBS. In one or more embodiments, the surface roughness of gold is calculated using FIJI ImageJ on optical images collected at various stages of corrosion. As depicted in FIG. 26D, the surface roughness is monitored every 0.5 minutes for 5 minutes from the initial stage, then every 1 minute from the 5-minutes stage to the 9-minutes stage.

As depicted in FIG. 26A, in one or more embodiments, a sharp increase in surface roughness is observed in the first 1 minute right after the crevice corrosion is initiated. In one or more embodiments, this is consistent with the amperometry study that most of the electrochemical corrosion happens within 1 minute under this potential. In one or more embodiments, the increase in roughness indicates the exfoliation of gold layer from the wafer, which is also observed in the microneedle corrosion from a SOP. In one or more embodiments, a significant part of gold layer is not directly oxidized but exfoliated during the electrical triggers. In one or more embodiments, this corresponds with experiments where gold film is used as the control gate for implanted drug reservoir.

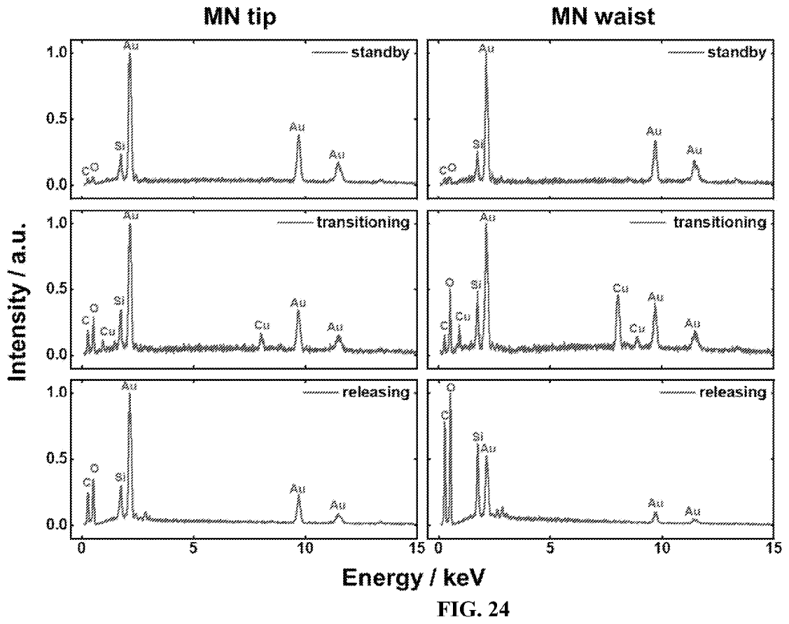

To characterize the electrical trigger process, two different areas on an SOP microneedle are selected, defined as the tip area and the waist area as shown in FIG. 16D. In one or more embodiments, the structural difference between the two areas is the surface curvature, with the former of 1.37 mm⁻² and the latter close to 0. In one or more embodiments, the waist area stands for the major surface of the microneedle, where most of the drugs are released through. In one or more embodiments, the energy-dispersive X-ray spectroscopy (EDXS) mapping is carried out on three stages of microneedles from both areas, as a semi-quantified surface analysis depicted in FIG. 24. In one or more embodiments, oxygen, carbon, and gold are selected as elements of interest, where oxygen and carbon show the exposed polymer body while gold shows the encapsulated surface of the microneedle. In one or more embodiments, the ratio quantification is based on the relative weight percentage of only these three elements despite the existence of other elements.

As shown in FIG. 16B to FIG. 16C, both the tip and the waist areas show similar trends in element relative ratios. In one or more embodiments, an increase in oxygen, carbon, and a decrease in gold (all by weight %) are observed. In one or more embodiments, the waist area (which stands for the main body of an microneedle) shows a more remarkable change in element constituents, indicating a more complete corrosion of triggered crevice for the gold layer. In one or more embodiments, the tip part of microneedle is still partially capped by small amount of gold at the releasing stage. In one or more embodiments, this part of gold layer is isolated during the crevice corrosion process and accounts for the rather high remaining gold showed in FIG. 16B. In one or more embodiments, the Au left covering the tip-area is too small to hinder the overall drug release from the entire microneedle. In one or more embodiments, silicon and copper are also observed in EDXS mapping, which comes from PDMS residuals and conductive wires used during the experiment.

FIG. 16G depicts the thermal characterization of SOP during the electrochemical corrosion to validate the thermal safety. An example experiment uses an microneedle array (for example, with a length of 1.2 mm and having a gold coating with a thickness of 150 nm as an electrically triggerable membrane) connected to a 2.5-V DC power to undergo crevice corrosion in 1×DPBS. In one or more embodiments, the temperature of microneedle array with and without current is recorded by a FLIR thermometer in 25° C. environment. In one or more embodiments, the results show no obvious change in temperature during the electrochemical corrosion, which indicates a low possibility of tissue damage from heat effects.

FIGS. 17A-17G depict wireless control of a SOP via near-field communication. FIG. 17A depicts optical images of microneedles with Au and without Au coating. In one or more embodiments, the Au layer remains stable in an artificial tissue (for example, having 0.5% agar in 1× Dulbecco's phosphate-buffered saline) for more than 10 days with no significant degradation. FIG. 17B depicts the encapsulation profile of a 100-nm Au layer on a 1.2-mm microneedle in 40° C. in 1× Dulbecco's phosphate-buffered saline. In one or more embodiments, the same parameters apply for FIGS. 17C-17G.

In one or more embodiments, the release of Rhodamine B (for example, 0.3% loaded) from an Au coated and uncoated microneedles is monitored and compared. FIG. 17C depicts an example optical image of the wireless SOP. FIG. 17D depicts an example power stability measurement of the wireless SOP. In the example shown in FIG. 17D, line 1710 depicts the input AC signal (for example, having a peak-to-peak value of 4 V with 39 MHz), line 1730 depicts rectified received signal (for example, 3.4 V with a stand deviation (s.d.) of approximately 0.2 V), and line 1720 depicts regulated output signal (for example, 2.6 V with a s.d. of approximately 0.1 V).

FIG. 17E provides an example component illustration of the wirelessly powered SOP depicted in FIG. 17C, featuring a receiving coil (shown as “Rec. coil” in FIG. 17E), a full-bridge rectifier (shown as “rectifier” in FIG. 17E), a 2.5-V regulator (shown as “regulator” in FIG. 17E), a counter electrode (shown as “CE” in FIG. 17E), and a gold-coated microneedle array (for example, each microneedle has a length of 1.2 mm and the gold coating with a thickness of 150 nm as an electrically triggerable membrane). In one or more embodiments, the counter electrode uses another Au-coated microneedle array of the same parameters.

FIG. 17F depicts example frequency matching characterization of the wireless power transfer. The dots 1750 depict the output voltage after rectification without regulation and the dots 1740 depict the output voltage after rectification and with 2.5-V regulation. In one or more embodiments, a 10-V peak-to-peak input signal is used and the working range is from 32-40 MHz is 36 MHz. In one or more embodiments, the potential rectification achieves stable bias around 2.5 V as the electrical trigger. FIG. 17G depicts an example corresponding circuit diagram of the SOP, with labels matching those in FIG. 17E. However, the inductive coil (Ind. coil) is not presented in FIG. 17E.

FIG. 17A depicts example encapsulation performance of gold coating on microneedles. In one or more embodiments, Rhodamine B (which is a fluorescent dye) is loaded into the microneedle patch (for example, having 0.3% in weight and approximately 90 ng per microneedle) to simulate small-molecule drugs, which can be subsequently quantified by UV-Vis spectroscopy.

FIGS. 17A-17B depict an example release profile of Rhodamine B from a bare microneedle patch (for example, with 1.2 mm in length) and an microneedle patch (for example, with 1.2 mm in length) with a 100-nm gold coating. The example release study is carried out in 40° C. 1×DPBS. Significant fading on the microneedle patch without gold coating as shown FIG. 17A and the obvious increase in absorbance without gold coating shown in line 1740 of FIG. 17B indicate a successful release of dye into the biofluids. In one or more embodiments, the average release rate of Rhodamine B in an hour is calculated as approximately 343 ng/min. In contrast, the example controlled experiment shows no significant change in absorbance (depicted in line 1750 in FIG. 17B), suggesting excellent protection of encapsulated drugs from releasing.

In one or more embodiments, the stability of gold coating is analyzed in a 0.5% agar model to better simulate the mechanical properties of animal tissue. In one or more embodiments, the microneedle array (for example, having a length of 1.2 mm and with a gold coating of 150 nm in thickness as an electrically triggerable membrane) remains stable during a 2-week soaking test in the agar model without significant changes in shape or surface morphology, as depicted in FIG. 17A.

FIG. 17C demonstrates an example wireless design of a SOP. In one or more embodiments, an external power source such as a signal generator is connected to an inductive coil to provide a high-frequency (at approximate MHz level) alternating current (AC). In one or more embodiments, the inductive coil is paired with the receiving coil on the device to achieve wireless power transfer via magnetic resonance coupling. In one or more embodiments, the AC current is then converted to direct current using a full-bridge rectifier. In one or more embodiments, the DC is then regulated by a 2.5-V regulator to provide a stable potential that facilitates the electrochemical crevice corrosion of gold layer on microneedles.

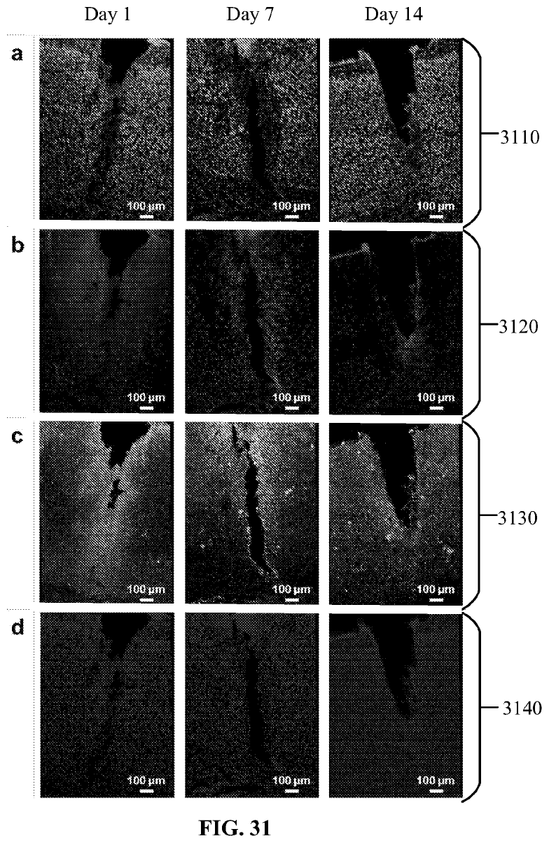

An example configuration of the wireless SOP is illustrated in FIG. 17E. In one or more embodiments, the wireless patch comprises a receiver coil for energy harvesting, a full-bridge rectifier for AC-DC transformation, a regulator for the stable output voltage (for approximately 2.5 V), and the microneedle array (for example, with a length of 1.2 mm and having a gold coating with a 150 nm thickness as an electrically triggerable membrane) with another microneedle array of the same parameters as the counter electrode (CE). The microneedle array is coupled with a 0.1-μF capacitor to constitute a low-pass filter, which improves the output stability. An equivalent circuit diagram is provided in FIG. 17G, including the external power source connected with an inductive coil. FIG. 31 shows an example performance and impedance analyses of this wireless SOP.

In one or more embodiments, the optimal signal input is determined as around 15 V peak-to-peak at 15 MHz. In one or more embodiments, the measurements shown in FIG. 17D validate the output stability of wireless SOP with the regulator. In one or more embodiments, the final output power signal is stabilized at around 2.5 V with a standard deviation of approximately 0.1 V, which ensures precise control of crevice corrosion for initiating drug release. As depicted in FIG. 17F, in one or more embodiments, the optimal frequency of the input signal is determined to be around 36 MHz.

In the example shown in FIG. 17G, the microneedles (“MN Patch”) and the counter electrode are connected to the output ends of the regulator. In such an example, the controller may control the

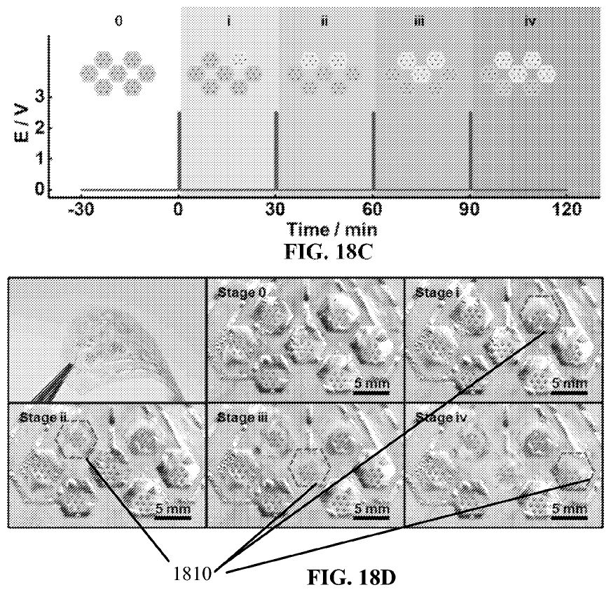

FIG. 18A to FIG. 18D illustrate characterization of spatiotemporal control of drug release. FIG. 18A depicts an example schematic illustration of a multi-domain SOP, with a zoom-in view of a microneedle domain at the releasing stage. FIG. 18B depicts an example stepwise release profile of a multistage drug release simulated by Rhodamine B. For example, a four-step release of Rhodamine B is electrically triggered at 0 minute, 30 minutes, 60 minutes, and 90 minutes (corresponding to labels i, ii, iii, iv, respectively in FIG. 18B). In this example embodiment, the SOP has microneedles with a height of 1.2 mm, coated with a 150-nm gold layer, and protected by a 10-μm PDMS layer.

FIG. 18C depicts an example schematic illustration showing the sequential electrical-triggering schedule on the multi-array SOP. In one or more embodiments, the electrical triggering uses direct current voltage 2.5 V for approximately 30 seconds. FIG. 18D depicts example optical images of the multi-domain SOP undergoing a sequential electrical trigger. In one or more embodiments, the domain triggered at each stage is labeled by hexagonal dashed frames. In one or more embodiments, the images from Stage 0 to Stage iv correspond to FIG. 18B and FIG. 18C.

In some embodiments, to further investigate the temporal and spatial controllability of electrical triggering on the microneedle patch depicted in FIG. 18A, an example multi-domain SOP is provided to realize the stepwise on-demand release. In one or more embodiments, the multi-domain SOP consists of 7 domains of microneedle arrays (each has a length of 1.2 mm and with a gold coating of 150 nm in thickness as an electrically triggerable membrane). In one or more embodiments, the Au layer on PLGA patch is patterned by laser ablation to enable separate triggering of individual microneedle domains. In one or more embodiments, a thin layer (for example, approximately 10 μm) of PDMS is then applied onto the patch except for the hexagonal microneedle regions to protect Au interconnects from dissolving during electrical triggering.

FIG. 18C depicts an example electrical triggering schedule for four of the seven microneedle domains for the SOP loaded with Rhodamine B (for example, 0.3% by weight) during its immersion in 65° C. 1×DPBS as an accelerated study.

In one or more embodiments, the electrical triggers use 2.5-V DC bias for 30 seconds at every 30 minutes interval of immersion. In one or more embodiments, following each interval, the environmental fluids are immediately sampled to allow quantitative estimation of drug-release dosage using UV-Vis spectroscopy, as shown in FIG. 18B. In one or more embodiments, the measurements show a multi-step increases in spectral absorbance, indicating stepwise increase of drug dosage and confirming the on-demand release of drug at desired time (0 minutes, 30 minutes, 60 minutes, and 90 minutes in FIG. 18B). FIG. 18D demonstrates an example staged release of microneedle domains by selectively dissolving the Au encapsulation layer with an electrical trigger. The dashed line 1810 circles the specific microneedles domain triggered from each stage. The results confirm that the example SOP realizes both temporal and spatial control of drug release using digital electrical triggers.

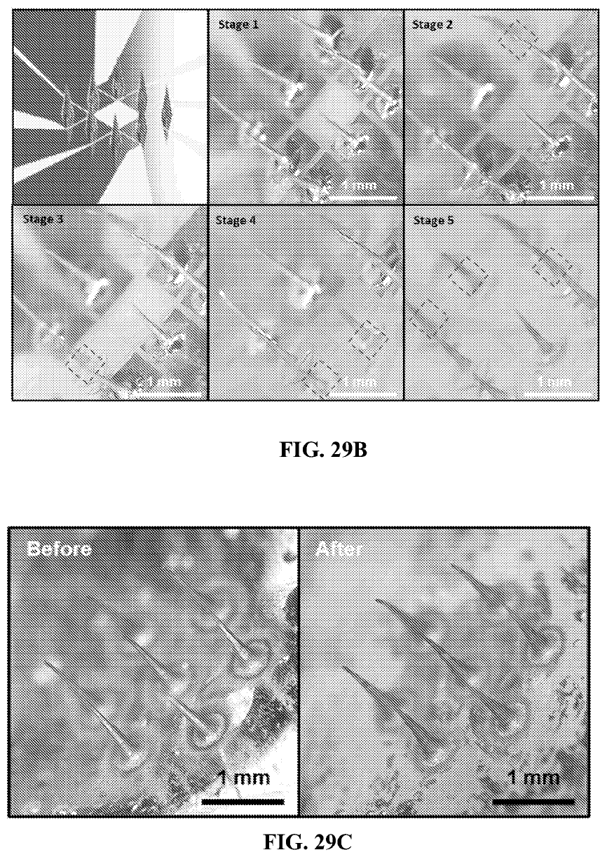

An example experiment depicted in FIGS. 29A-29C shows the possibility of ultrafine spatial control of drug release. In this experiment, a miniaturized SOP with a single domain is designed, consisting of 8 microneedles (for example, each with a length of 1.1 mm and having a gold coating of 150 nm in thickness as an electrically triggerable membrane) with between 1 mm and 3 mm in spatial separation. As illustrated in FIG. 29A, with specific design of Au circuits, each individual microneedle in the domain can be triggered separately. FIG. 29B shows that each microneedle can be electrically triggered (for example, by a 2.2-V DC) within 15 seconds without any interference to adjacent microneedles. In one or more embodiments, the spatial resolution of release control for SOP are primarily dictated by patterning techniques on the Au layer.

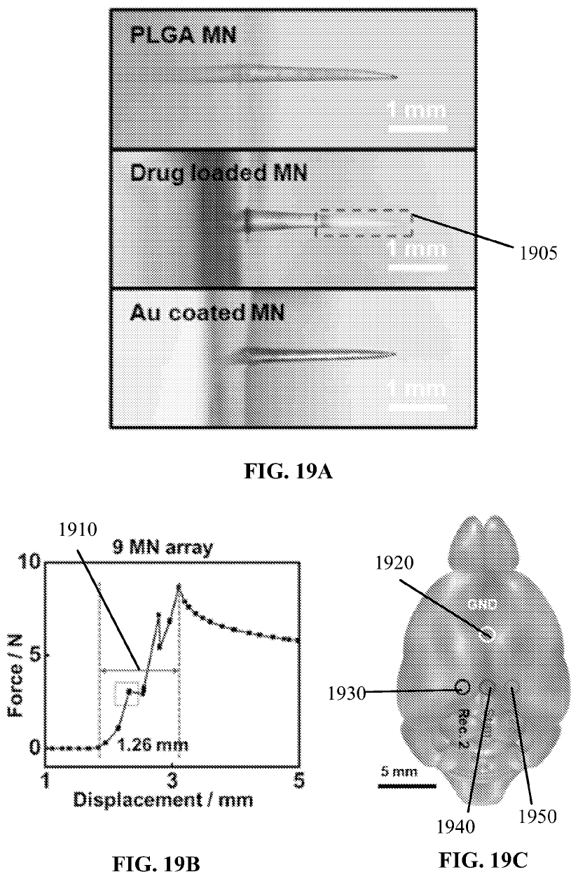

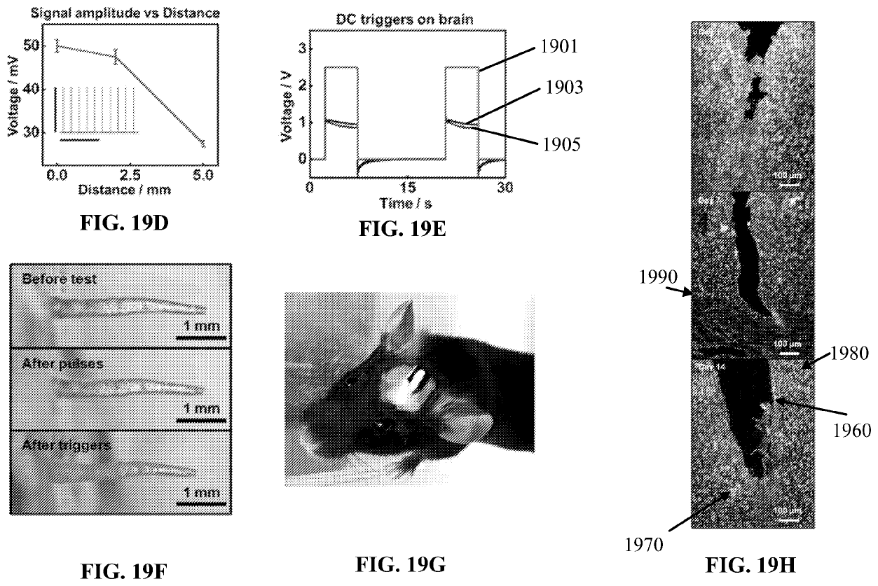

FIG. 19A to FIG. 19H depict an example in vivo demonstration of SOP. FIG. 19A illustrates example optical images of an intracranial microneedle (with a height of 3 mm) at various fabrication stages, including 1) PLGA needle base; 2) microneedle loaded with melatonin (20%); 3) microneedle coated with 150-nm gold. In one or more embodiments, the drug concentrated region is circled with the frame.

FIG. 19B illustrates the measured force-displacement curve of a microneedle array (for example, comprising 9 needles, each with a height of 1.2 mm) during a fracture test. In one or more embodiments, the first fracture point is circled by frame 1910 and the displacement in contact is labeled and measured as 1.26 mm.

FIG. 19C depicts an example schematic illustration of a brain model indicating deployment location of SOP in the in vivo study. Circles 1940 and 1920 label positions of the SOP microneedle and counter electrode (for example, Pt wire), respectively. Circles 1950 and 1930 label the positions of two separate recording electrodes (for example, Pt wire).

FIG. 19D depicts measured pulsatile triggers generated from the SOP microneedle at various distances to the microneedle. In one or more embodiments, a pulse signal (for example, a 50 mV signal with 1 Hz and 10 ms in width) is applied by a microneedle electrode and recorded by a Pt wire at approximately 0.1 mm, 2.0 mm, and 5.0 mm. In one or more embodiments, the signal for stimulation is shown in vertical lines with a scale bar of 5 seconds and 50 mV.

FIG. 19E depicts example measured square wave triggers 1901 generated from the SOP microneedle at various distances to the microneedle. In one or more embodiments, the triggers 1901 are delivered by the gold coated microneedle (for example, having a length of 3 mm and with a gold coating of 150 nm in thickness as an electrically triggerable membrane) as a 2.5-V 5-s square wave periodically. In one or more embodiments, the two recording electrodes are around 5-mm away from the microneedle, and the signals from the recording electrodes are shown as line 1903 and line 1905. FIG. 19F depicts example optical images of the microneedle (for example, with a length of 3 mm) before the test, after pulses triggers, and after square wave triggers. FIG. 19G depicts an example mouse after SOP deployment. FIG. 19H depicts example immunohistochemical staining images of the recovery process after microneedle implantation. The example combined images include Nissl bodies (neurotrace 1990), astrocytes (glial fibrillary acidic protein (GFAP)) 1960, activated microglia (Ibal) 1970 and DNA (4′,6-diamidino-2-phenylindole (DAPI) 1980) shown in FIG. 19H.

Beyond the clinical applicability for transdermal drug delivery, the SOP also show impactful utility in facilitating animal behavior study. In an example experiment, intracranial delivery of melatonin using SOP for animal sleep study is demonstrated. Melatonin, a hormone that is naturally produced in the brain by the pineal gland, plays a crucial role in regulating the sleep-wake cycle, while also participating in some other regulations such as, but not limited to, blood pressure and body temperature. The high spatiotemporal controllability of melatonin release offered by the SOP may open up new opportunity to understand regional brain responses to melatonin and study pathology of narcolepsy. Loading Melatonin into microneedles of the SOP follows the solution fabrication method described in FIG. 20.

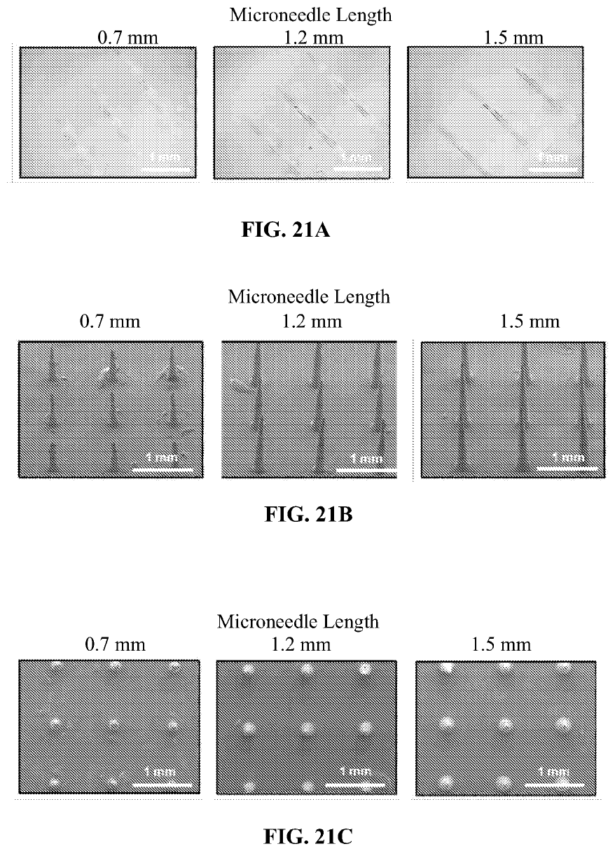

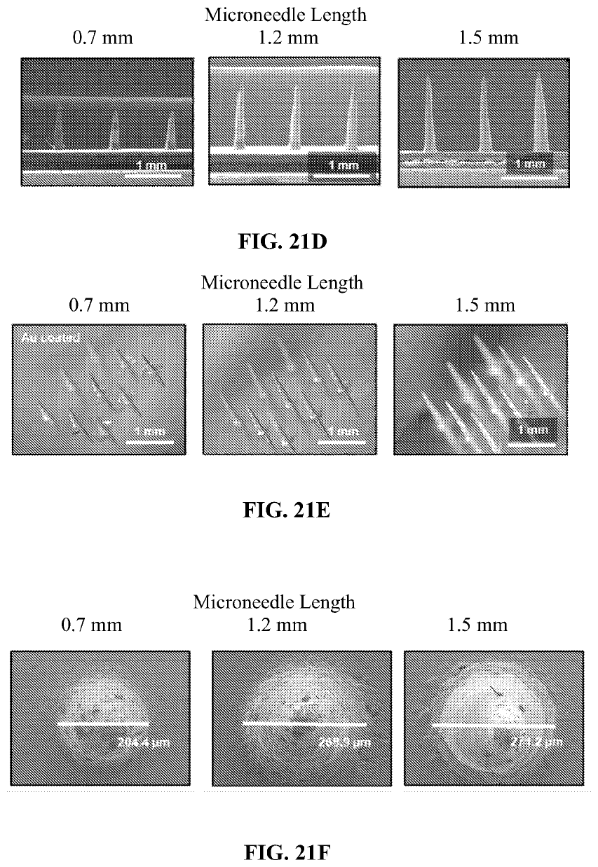

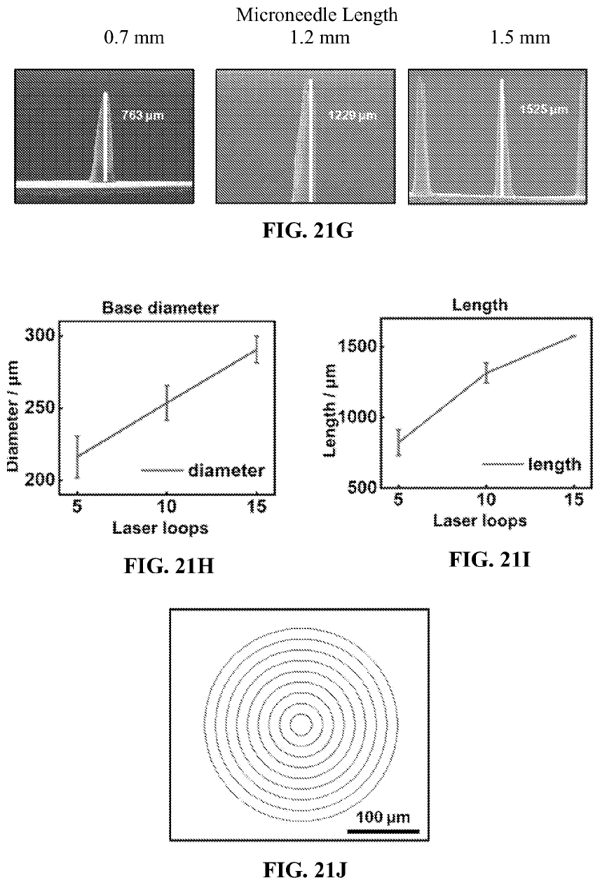

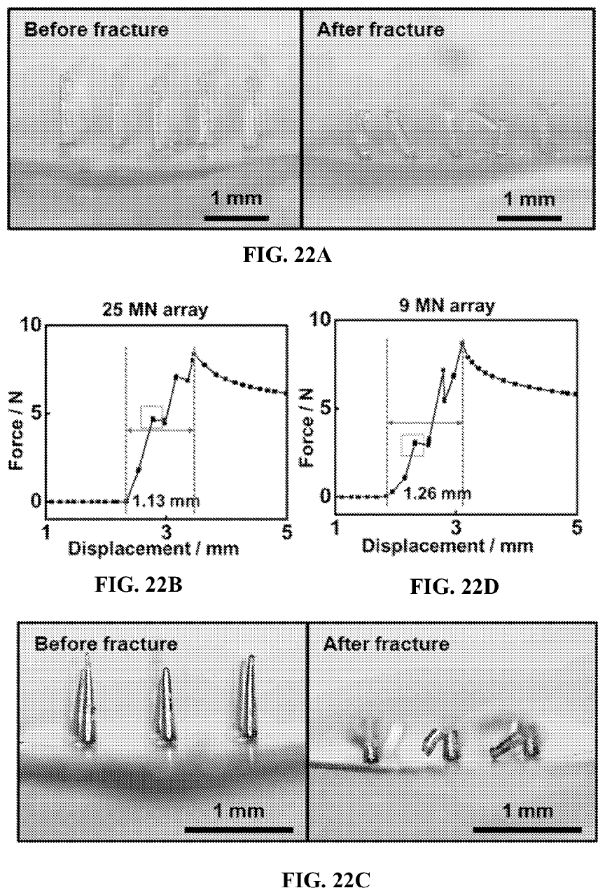

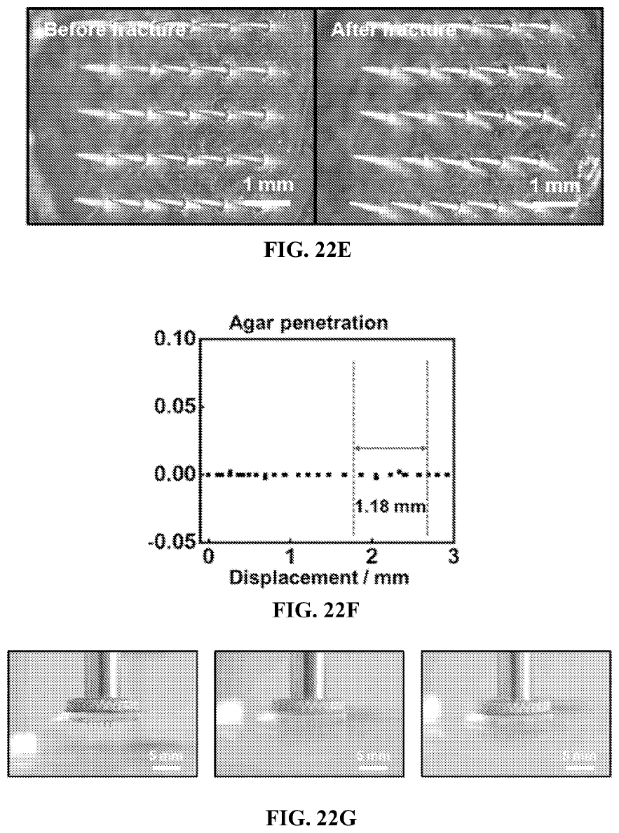

In one or more embodiments, melatonin that dissolves in acetone can be mixed with the precursor PLGA solution, which ensures the loading dosage for each microneedles. In one or more embodiments, as depicted in FIG. 19A, the 3-mm microneedles are fabricated with the PLGA-melatonin ratio from 10:1 to 2:1. In addition, in one or more embodiments, the drug can be concentrated on certain part of the microneedle, as shown by the dashed line 1905 in FIG. 19A. Based on the mixing ratio, in one or more embodiments, the payload of melatonin per microneedle is estimated to be between 22.2 to 81.7 μg, which is comparable to recommended dosages for mice (which is 4 to 20 mg/kg). In one or more embodiments, the drug payload is modulated by loading different PLGA solution during the mold casting procedure. In one or more embodiments, the mechanical property of the 3 mm-tall microneedles using for intracranial delivery is characterized by a fracture test depicted in FIG. 22A to FIG. 22G. In one or more embodiments, the ultimate strength of PLGA microneedle (which is 1.2-mm in length) is determined by the first fracture point in the force-displacement graph, as labeled by the frame 1910 of FIG. 19B. In one or more embodiments, the fracture point (recognized by a sudden drop in measured force) corresponds to the initial fracture of the microneedles, which is followed by multiple subsequent fractures appearing on different locations of the microneedles shown in FIG. 22B and FIG. 22D. In one or more embodiments, the maximum mechanical strength is derived to be 118 MPa, which is rigid enough for human skin penetration. In one or more embodiments, the calculation considers the pressure measured at the first fracture point with the contact area of the microneedle being approximated based on the tip diameter (around 30 μm in FIG. 21F).

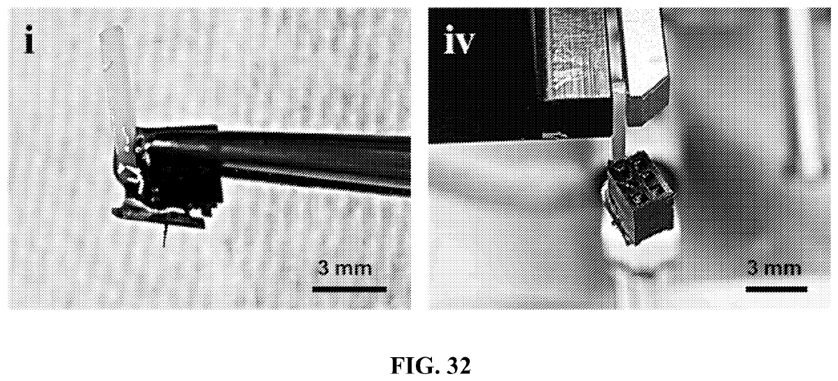

In one or more embodiments, FIG. 19G deploys the melatonin-loaded SOP in live animal models and shows possibilities for active control of melatonin releasing to the deep-brain regions in the parietal lobe as the animals move in a cage environment. In one or more embodiments, to ensure device stability, the SOP is coupled with a custom head stage that can be firmly mounted onto mice heads, as shown in FIG. 32. FIG. 19H shows an example immunohistochemistry analysis of brain tissues from the mice at various recovery stages post to SOP implantation. In one or more embodiments, on day 1 post-implantation, the brain tissues in close proximity to the SOP microneedle show structural damage resulted from mechanical forces during intracranial surgery, which is typical for general brain implantation. In one or more embodiments, as the mice recover from the implantation, the levels of GFAP 1960 and IBA 1970 show a significant decrease in concentration and their staining range, indicating excellent biocompatibility of SOP. In one or more embodiments, the tissue in contact with the microneedle becomes smoother, with fewer rough edges. In one or more embodiments, an obvious increase in neuron regeneration (neurotrace 1990) can be observed indicating good recovery from implantation surgery.

In one or more embodiments, the in vivo animal model is used to characterize the SOP functional performance. In one or more embodiments, an additional two recording electrodes are inserted adjacent to the location of SOP implantation, as shown in FIG. 19C. In one or more embodiments, a set of DC electrical triggers (for example, having 5 second in duration and 2.5 V) is first delivered and recorded (for example, at 5 mm from the stimulation electrode) as shown in FIG. 19E. In one or more embodiments, the crevice corrosion of Au layer (having a thickness of 150 nm) on the microneedle can be completed by applying 5 to 7 times the 5-s triggers to allow melatonin release, as shown in FIG. 19H.

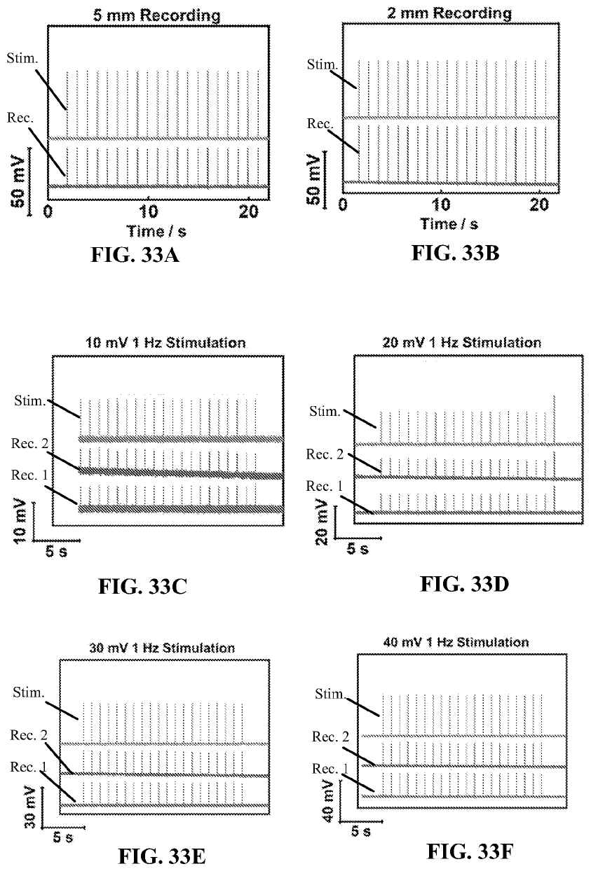

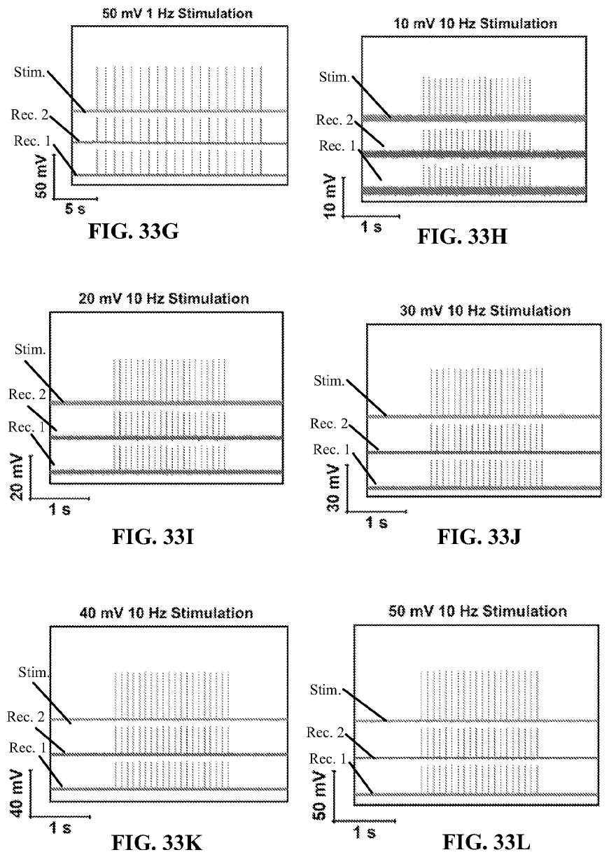

In one or more embodiments, a series of short pulse stimulation experiments are examined, as shown in FIG. 33A to FIG. 33L. In one or more embodiments, the pulse signals vary from 10 mV to 50 mV in amplitude and 1 Hz (with a duration of 10 ms) or 10 Hz (with a duration of 1 ms) in frequency. In one or more embodiments, the relationship between signal amplitude and recording distance is also studied based on 10-ms pulses of 50 mV as depicted in FIG. 19D, and proved to mainly affect a 5-mm area. In one or more embodiments, no significant changes are observed on the Au layer of microneedle after short pulses, indicating electrical signals generated from neuron cells induce negligible damage to the Au protection layer of the SOP. In one or more embodiments, the results demonstrate the Au-coated microneedles can also be used as stimulation electrodes for delivery of low-amplitude (for example, 10 to 50 mV) pulsatile signals as depicted in FIG. 19F, which may serve as a strategy for neuronal regeneration.

### 8. Example Fabrication of SOP

Various examples of the present disclosure provide example methods, devices, and systems for fabricating various types of microneedle patches, including, but not limited to, normal microneedle patches, melatonin-loaded microneedle patches, and Rhodamine B loaded microneedle patches.

For normal microneedle patches, an example general method of an example microneedle fabrication is demonstrated in FIG. 20. In one or more embodiments, an example fabrication method includes first fully curing 50 grams of PDMS (for example, Sylgard 184 from Dow Corning) in a glass petri dish at 60° C. for 2 hours with 5 grams of its corresponding curing agent. In one or more embodiments, a negative microneedle mold is then patterned on the cured PDMS by a UV laser ablation system. In one or more embodiments, the microneedle molds are fabricated with different depth ranging from 0.5 to 3.5 mm, a base diameter of around 0.25 mm, and an inter-needle spacing of 1 mm. In one or more embodiments, the depth of the microneedle mold can be controlled by tunning the loops and power of UV laser ablation. In one or more embodiments, the UV ablation is followed by acetone sonication for at least 5 minutes to clean up the surface of the PDMS negative mold. In one or more embodiments, a PLGA solution (with 10 wt. % in acetone) is then drop casted on the PDMS negative mold in the petri dish. In one or more embodiments, the PLGA-covered mold is heated at 45° C. and with a pressure of 60 to 160 mmHg for around 2 minutes to let the PLGA solution fill in the mold and evaporate. In one or more embodiments, the entire PDMS mold is capped by another petri dish to slow down the evaporation of acetone. In one or more embodiments, the evaporation process is followed by a refill of PLGA solution. In one or more embodiments, the evaporation-refilling cycle was conducted 10 to 20 times to provide enough PLGA for the microneedle patch, with the thickness from 0.6 to 1.2 mm. In one or more embodiments, the PLGA-covered mold is then kept in the oven at 45° C. and 1 atm for at least 8 hours to dry up the surface. In one or more embodiments, the mold is then frozen at −20° C. for at least 30 minutes to harden the PLGA patch, which is subsequently extracted from the mold. In one or more embodiments, the free-standing PLGA patch is allowed to further dry up both sides at 45° C. and 1 atm for another 24 hours, then trimmed by UV laser ablation. In one or more embodiments, the hardened and dry PLGA patch is eventually deposited with a layer of gold (usually 15 nm in thickness) by sputter coating (for example, PVD 75 sputterer by Kurt J. Lesker). The Gold traces are patterned by an IR laser ablation system.

Various examples of the present disclosure also provide example methods, systems, and apparatuses for fabricating melatonin-loaded microneedles. In one or more embodiments, the single microneedle loaded with melatonin is fabricated by the same mold casting method described before. In one or more embodiments, the melatonin is mixed with PLGA at a ratio by weight from 1:10 to 1:2. In one or more embodiments, the mixture is then dissolved in acetone at a 1:10 ratio by weight. In one or more embodiments, the subsequent fabrication is conducted at 30° C. instead of 45° C.

Various embodiments of the present disclosure also provide example methods, systems, and apparatuses for fabricating Rhodamine B-loaded microneedles. In one or more embodiments, the Rhodamine B loaded microneedles are fabricated in the same way as a normal microneedle patch. In one or more embodiments, Rhodamine B is dissolved in the acetone solution of PLGA (usually at 1/300 ratio by weight). In one or more embodiments, the subsequent fabrication procedures are the same as those described above.

### 9. Example Corrosion of Microneedles

In accordance with various embodiments of the present disclosure, example Kinetic characterization of electrochemical corrosion is demonstrated.

In one or more embodiments, a microneedle patch coated with gold is connected to a piece of graphene tape. In one or more embodiments, the peripheral area of microneedle patch (except for needles) and the graphene tape are protected by PDMS from corrosion, with an opening area of 0.5 by 0.5 cm². In one or more embodiments, an amperemeter (for example, NI-USB 4065 from National Instruments) is applied to monitor the current density as compared to time. In one or more embodiments, the experiment is carried out in a two-electrode system, where the counter electrode is graphene tape. In one or more embodiments, a power source (for example, SPD3303X-E by Siglent) is used to provide constant voltage between cathode and anode, where the anode is connected to the gold-coated microneedle patch to provide oxidative potential. In one or more embodiments, the microneedle patch with graphene tape is coated with PDMS (for example, approximately 10 μm in thickness) to protect the exposed surface except for the microneedle regions. In one or more embodiments, the electrochemical corrosion is carried out in standard environment (for example, in 1×DPBS from Cornings) to mimic the body fluid. In one or more embodiments, I-t curves are obtained at 2.2 V, 2.4 V, 2.6 V, 2.8 V, and 3.0 V. In one or more embodiments, kinetic characterization is also carried out for Mo-coated microneedle arrays in the same way.

In accordance with various examples of the present disclosure, example dye release resulting from electrochemical corrosion are also illustrated. For example, examples of the present disclosure illustrates free release without encapsulation, encapsulated release, and on-demand stepwise release.

In an example free release without encapsulation, Rhodamine B (for example, from thermo scientific with a mass ratio versus PLGA as 1:300) is mixed with PLGA and dissolved together in the acetone solution. In one or more embodiments, the fabricated microneedle patch is immersed in a petri dish containing 20 mL of deionized water (for example, from HAVENLAB) at 40° C. constant temperature. In one or more embodiments, samples are taken for UV-Vis spectrometry since the microneedle patch is immersed, every 1 minute from 0 to 10 minutes, every 5 minutes from 10 to 30 minutes, and then at 60 minute. In one or more embodiments, the UV-Vis absorbance is characterized by a UV-Vis spectrophotometer (for example, VWR-10037, VWR) from 800 to 300 nm, with an interval of 1 nm. In one or more embodiments, the samples are returned back to the petri dish immediately after characterization to maintain a constant volume.

In an example encapsulated release, a Rhodamine B loaded microneedle patch is deposited with a 100-nm gold layer on the side with needles to encapsulate the PLGA and dye. In one or more embodiments, the back side of microneedle patch is fixed and encapsulated into PDMS to prevent exposure to water. In one or more embodiments, the patch is immersed in a petri dish with 20 mL of 1×DPBS at 45° C. In one or more embodiments, UV-Vis spectra are characterized in the same way as mentioned above.

In an example on-demand stepwise release, a Rhodamine B loaded microneedle patch is sputter deposited with a 150-nm gold layer on the side with needles, then patterned by IR laser to generate gold traces. In one or more embodiments, the gold electrodes are connected by silver paste (for example, 8331D from MG Chemicals) to the constant voltage power source. In one or more embodiments, the entire PLGA patch is then encapsulated with PDMS except for the needle region. In one or more embodiments, the patch is immersed in a petri dish with 20 mL of DI water at 60° C. In one or more embodiments, the electrochemical corrosion of gold is triggered by a 2.5 V constant voltage within 20 seconds. In one or more embodiments, the dye release of 4 microneedle arrays is subsequently triggered every 30 minutes. In one or more embodiments, UV-Vis spectra are characterized in the same way as mentioned above, every 5 minutes from 0 to 120 minutes. In one or more embodiments, samples are taken at certain intervals for UV-Vis absorbance characterization and returned back to keep the volume constant.

Various examples of the present disclosure demonstrate example surface profilometry of corrosion.

For example, a silicon wafer (for example, 100 facet by UniversityWafer) is deposited with a 150-nm gold layer by sputtering and divided into 4 cm² squares. In one or more embodiments, the gold-coated side of the wafer is connected to a DC power source by graphene tape. In one or more embodiments, the peripheral area of the wafer square is encapsulated by PDMS with a 1 cm² window exposed in the center, as illustrated in FIG. 26C. In one or more embodiments, the wafer square is immersed in 10 mL 1×DPBS and the electrochemical corrosion is triggered by a 2.5 V constant voltage. In one or more embodiments, optical images of the wafer square are captured by a microscope (for example, S9i from Leica) starting from the beginning. In one or more embodiments, the time interval of images is 0.5 minutes from 0 to 5 minutes and 1 minute from 5 to 9 minutes. In one or more embodiments, the obtained images are cropped to leave only the exposed window in the center and further analyzed by FIJI ImageJ. In one or more embodiments, arithmetic mean roughness (Ra) and root mean square roughness (Rq) are calculated for each image by the roughness analysis module.

Various examples of the present disclosure demonstrate thermal effects associated with example implementations.

In one or more embodiments, a 150-nm gold-coated microneedle array is placed in a petri-dish and immersed in 10 mL 1×DPBS at room temperature (for example, 25° C.). In one or more embodiments, the microneedle array is soldered with a copper wire and connected to the DC power source. In one or more embodiments, the infrared radiation image is recorded by a thermal camera (for example, by ETS320 from FLIR). In one or more embodiments, for the first 1 minute, the temperature of the microneedle array is recorded without any voltages applied. In one or more embodiments, for the following 1 minute, the microneedle array is triggered by a 2.5 V constant voltage. In one or more embodiments, temperature data points were taken every 5 seconds from the videos.

Various examples of the present disclosure demonstrate example elemental analyses associated with corrosions.

In one or more embodiments, the electrochemical corrosion of microneedle arrays (for example, with a length of 1.2 mm and having a gold coating with a thickness of 150 nm as an electrically triggerable membrane) is conducted at 2.4 V in 1×DPBS. In one or more embodiments, the triggering time is 0.5 minutes and 2 minutes for two microneedle arrays, which stands for the “middle” and “after” stages of the corrosion procedure. In one or more embodiments, optical and SEM images are captured as shown in FIG. 23A and FIG. 23B.

In one or more embodiments, the EDXS element mapping is conducted by the Scanning Electron Microscope (for example, SEM from Hitachi S-4700). In one or more embodiments, 20 kV is applied under analysis mode at a selected 100 by 120 μm² area, and signal is collected for 400 seconds. In one or more embodiments, for microneedle samples at the releasing stage (which have little gold coating left) a 5-nm Pd layer was sputter coated before characterization to increase the surface conductivity. In one or more embodiments, the data analysis is automatically done by the INCA software (for example, from Oxford Instruments).

Various examples of the present disclosure demonstrates example mechanical strength measurements associated with example implementations of various embodiments.

In one or more embodiments, a force measurement system (by Mark 10, ESM 303) is implemented to study the mechanical strength of microneedles. In one or more embodiments, a microneedle array (e.g. 9 or 25-needle, 1.2-mm, with or without 150-nm gold coated) is attached to the top sample holder by glue and double-sided tapes, as shown in FIG. 22G. In one or more embodiments, the bottom sample holder is placed with a piece of glass to serve as a hard object. In one or more embodiments, both the glass and the microneedle array are placed as horizontally as possible. In one or more embodiments, the sampling rate of the force gauge is set as “as high as possible,” and the moving speed of sample holder is set as 13 mm/min, which is the lowest value. In one or more embodiments, the sample holder will gradually descend to a point where the microneedles are in contact with the glass, and stopped manually when the microneedle array is fully crushed (for example, as shown in FIG. 22A and FUG, 22C). Another soft contact experiment is also conducted under the same parameters except for the glass, which is replaced by 0.5% agar to mimic the brain (for example, as shown in FIG. 22E and FIG. 22F).

Various examples of the present disclosure demonstrates example finite element analysis of crevice corrosion associated with example implementations of various embodiments.

In one or more embodiments, the finite element analysis (FEA) of gold layer crevice corrosion on microneedles is simulated by COMSOL 6.1. In one or more embodiments, a model of microneedle pair (for example, with 1.2-mm and 3.5-mm in distance) is set up to simulate the exposed surface of cathode and anode in the fluid. In one or more embodiments, the tip of the microneedle is rounded into a hemisphere (for example, 100-μm in diameter) to facilitate convergence, while the bottom is set as a circle (for example, 270-μm in diameter). In one or more embodiments, the surface of one microneedle is set as the cathode boundary and the other is set as the anode boundary. In one or more embodiments, the entire simulation domain is set as a cubic (for example, 5-mm in length) space including the two microneedles. In one or more embodiments, to simulate the physical and chemical properties of 1×DPBS, water is chosen from the built-in database and used as the material of the cubic domain (not including microneedles). In one or more embodiments, the electroconductivity is set as 1.6 S/m, which is a typical conductivity from the manufacturer. In one or more embodiments, other surfaces of the model (except for the microneedle surfaces) are set as insulation in the boundary conditions. In one or more embodiments, stationary and transient simulations are conducted based on secondary current distribution, which considers overpotentials while assuming homogeneous electrolyte. In one or more embodiments, the anode equilibrium potential, according to literatures, is set as 1.83 V (Au⁺|Au), to describe the electrochemical force needed to trigger gold oxidation. In one or more embodiments, the cathode equilibrium potential is set as 0 V, and the electromotive force (which is the voltage of the external power source) is set as 2.5 V unless specified. In one or more embodiments, the model is automatically meshed at the ultrafine level under physics-controlled mode. In one or more embodiments, the current density distribution shown in FIG. 34A and the iso-potential surface shown in FIG. 34B to FIG. 34D are simulated at the starting point of the crevice corrosion, which is stationary. In one or more embodiments, the anodic polarization curve applying different electromotive force (between 2.0 V and 3.2 V) is simulated as shown in FIG. 34E. In one or more embodiments, transient simulation is conducted for the corrosion depth versus time, assuming the gold layer on microneedle is thick enough. In one or more embodiments, the corrosion depths from the starting time 0 second to 60 seconds is monitored every 10 seconds (as shown in FIG. 34F) and visualized in FIG. 34G.

Various examples of the present disclosure demonstrates example immunohistochemical analysis associated with example implementations of various embodiments.

In an example experiment, mice are given a lethal dose of pentobarbital sodium, followed by intracardial perfusion with 4% paraformaldehyde in PBS. In the example experiment, the brains are dissected, post-fixed for 24 hours at 4° C., and cryoprotected with a solution of 30% sucrose in 0.1 M phosphate buffer (pH 7.4) at 4° C. for at least 24 hours, and are fully submerged. In one or more embodiments, this is followed by cutting into 40-μm sections, washing three times in PBS, three 5-min incubations in 1 mg/ml⁻¹ sodium borohydride in PBS, then 1-hour incubations in 1% Triton-X-100 in PBS. In one or more embodiments, a blocking step is then performed using 5% donkey serum in 0.3% PBST for 1 hour. In one or more embodiments, brain sections are then incubated for approximately 16 hours at 4° C. in blocking buffer containing goat anti-GFAP from Santa Cruz Biotechnology (for example, 1:1000) and rabbit anti-Ibal of Fujifilm Wako (for example, 1:500). In one or more embodiments, sections are then transferred to a secondary antibody solution containing Alexa Fluor 647 donkey anti-rabbit IgG (1:1,000), Alexa Fluor 568 donkey anti-goat IgG (for example, 1:1,000) and Neurotrace 435/455 Blue Fluorescent Nissl stain (for example, 1:100) in 0.1% PBST for 1 hour at 24° C., with intermittent brief periods of shaking.

In one or more embodiments, sections are washed three times for 30 minutes each in 0.1% PBT, with 1 μM DAPI solution included on the third wash step. In one or more embodiments, after rinsing, slices are dried on a slide glass and cover slipped. In one or more embodiments, all brain slices are imaged with an Olympus FV3000 microscope. In one or more embodiments, all images were processed with the same settings using the Fiji software ImageJ.

FIG. 20 depicts an example schematic illustration of a fabrication process including stages: i. PDMS mold curing; ii. UV laser ablation of microneedle negative molds; iii. PLGA mold casting of PLGA solution; iv. PLGA microneedle patch extraction; v. gold deposition by sputtering; and vi. IR Laser patterning of gold.

FIG. 21A to FIG. 21J depict an optical and electron microscopy of microneedles of different dimensions. FIG. 21A depicts example optical images of PLGA microneedles. FIG. 21B depicts SEM of PLGA microneedles from the 45-degree perspective. FIG. 21C SEM of PLGA microneedles from the top perspective. FIG. 21D depicts SEM of PLGA microneedles from the horizontal perspective. FIG. 21E depicts example optical images of gold coated microneedles. FIG. 21F depicts an example measurement of the base diameter of microneedles. FIG. 21G depicts an example measurement of the length of microneedles. FIG. 21H depicts an example statistical analysis of the base diameter of different microneedles. FIG. 21I depicts an example statistical analysis of the length of different microneedles. FIG. 21J depicts an example laser pattern for UV ablation of a microneedle with the base diameter of 270 μm.

FIG. 22A to FIG. 22G depict an example mechanical characterization of microneedles. FIG. 22A depicts optical images of a 5 by 5 1.5-mm microneedle array before and after fracture test. FIG. 22B depicts an example mechanical testing curve for the 25-microneedle array, with the first mechanical failure circled in a red frame. In one or more embodiments, the contact distance was labeled as 1.13 mm. FIG. 22C depicts example optical images of a 3 by 3 1.2-mm microneedle array before and after fracture test. FIG. 22D depicts an example mechanical testing curve for the 9-microneedle array, with the first mechanical failure circled in a frame. In one or more embodiments, the contact distance is labeled as 1.26 mm. FIG. 22E depicts example optical images of a 5 by 5 1.5-mm microneedle array before and after agarose penetration test. FIG. 22F depicts an example mechanical testing curve for the 25-microneedle array on 0.5% agarose. In one or more embodiments, the contact distance is labeled as 1.18 mm. FIG. 22G depicts example optical images of the stages of agarose penetration test including before contact, partially penetrated, and fully penetrated.

FIG. 23A to FIG. 23D depict example optical and electron microscopy of microneedle at different stages of electrochemical crevice corrosion (including standby stage at 0 minutes, transitioning stage at 0.5 minutes, and releasing stage at 2 minutes). FIG. 23A depicts example optical images of microneedles from the standby stage to the releasing stage. FIG. 23B depicts example SEM images of microneedles from the standby stage to the releasing stage from the horizontal perspective. FIG. 23C depicts example SEM of microneedles from the standby stage to the releasing stage from the 45-degree perspective. FIG. 23D depicts example SEM images of microneedles from the standby stage to the releasing stage from the top perspective.

FIG. 24 depicts an example EDXS spectra of microneedles from different stages of electrochemical corrosion on two areas (including the top area and the waist area). In one or more embodiments, the corresponding elements of EDXS peaks are labeled. In one or more embodiments, stages of corrosion are labeled as standby, transitioning, and releasing, corresponding to the stages in FIG. 21A to FIG. 22G.

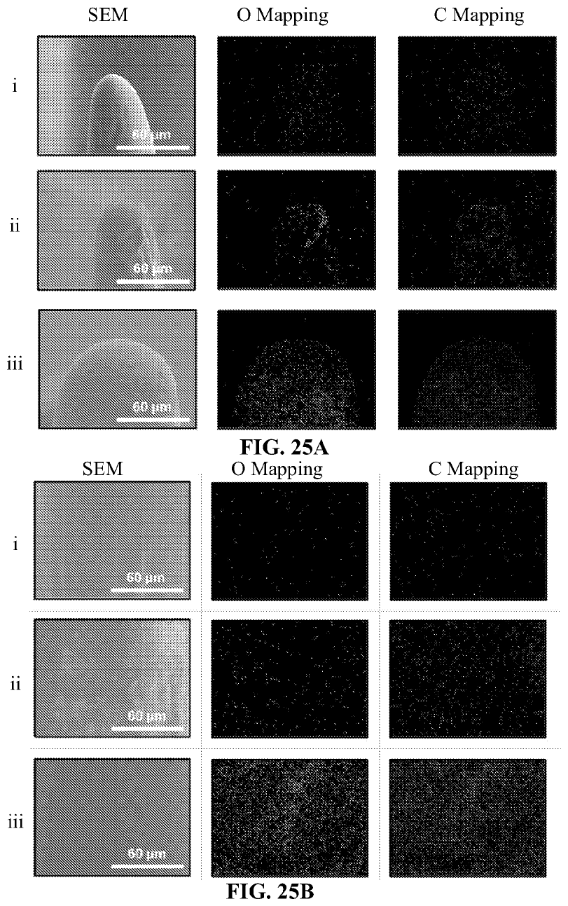

FIG. 25A to FIG. 25B depict example EDXS element mapping of oxygen and carbon from different stages of electrochemical crevice corrosion on two parts of the microneedle (including the tip and the waist). FIG. 25A depicts an example high magnification SEM image, oxygen, and carbon mapping of the tip area of microneedles from three stages that includes i. standby stage; ii. transitioning stage; and iii. releasing stage. FIG. 25B depicts the high magnification SEM image, oxygen, and carbon mapping of the waist area of microneedles from three stages: i. standby stage; ii. transitioning stage; and iii. releasing stage.

FIG. 26A to FIG. 26D depict surface profilometry of gold-on-wafer during electrochemical crevice corrosion. FIG. 26A depicts the roughness analysis of gold surface during corrosion with RMS roughness 2620 (R_{q}) and arithmetic roughness 2610 (Rₐ). FIG. 26B depicts an example schematic illustration of the experiment setup. In one or more embodiments, a two-electrode system is used in the 1×DPBS environment with a DC power source. In one or more embodiments, both the cathode and anode comprises a 150-nm gold layer. In one or more embodiments, the crevice corrosion is triggered by a 2.5-V constant voltage. FIG. 26C illustrates an example zoom-in view of the experimental device. In one or more embodiments, gold traces are connected to a power source by graphene tape and protected by PDMS (approximately 10 μm). In one or more embodiments, a 1 by 1 cm² windows on both cathode and anode are left open to allow for exposure. FIG. 26D depicts example optical images of the exposed gold region on anode during crevice corrosion, where the white areas are recognized as exfoliated gold.

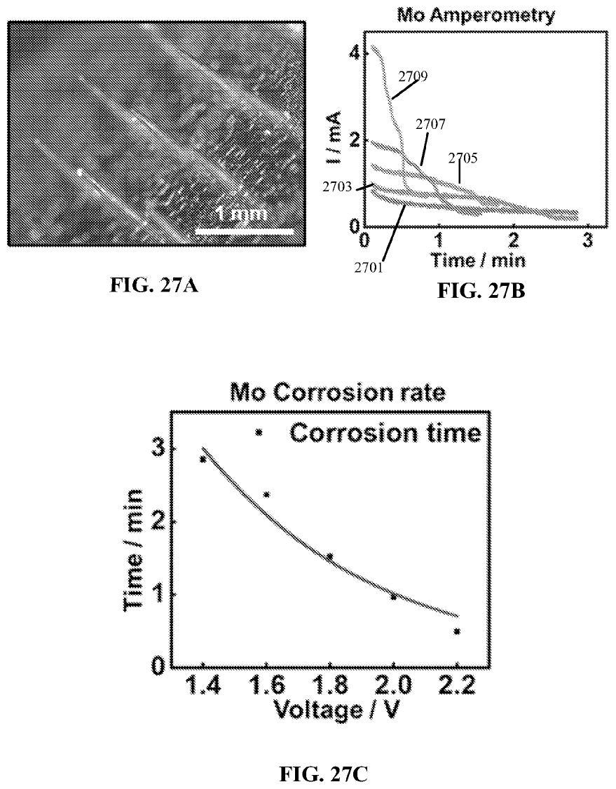

FIG. 27A to FIG. 27C depict an example characterization of electrochemical crevice corrosion of Mo-coated microneedles. FIG. 27A depicts an example optical image of a 3 by 3 100-nm Mo coated microneedle array (with a length of 1.2 mm). FIG. 27B depicts an example amperometry characterization of the electrochemical crevice corrosion of Mo layer on microneedles under different potentials in 1×DPBS. In particular, line 2701 corresponds to 1.4 V, line 2703 corresponds to 1.6 V, line 2705 corresponds to 1.8 V, line 2707 corresponds to 2.0 V, and line 2709 corresponds to 2.2 V. FIG. 27C depicts an example relationship between corrosion time and potential.

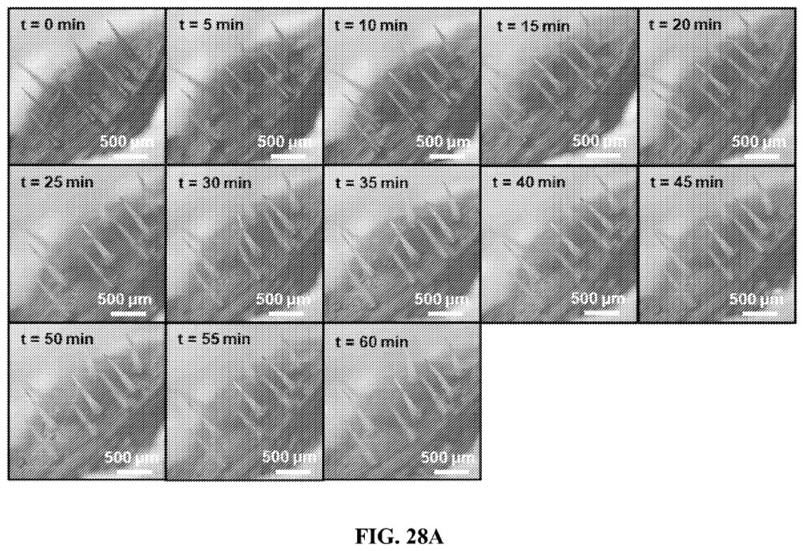

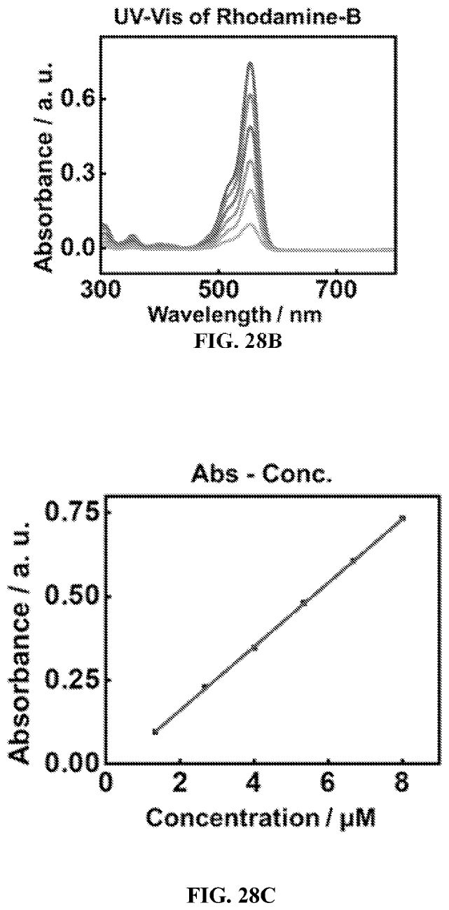

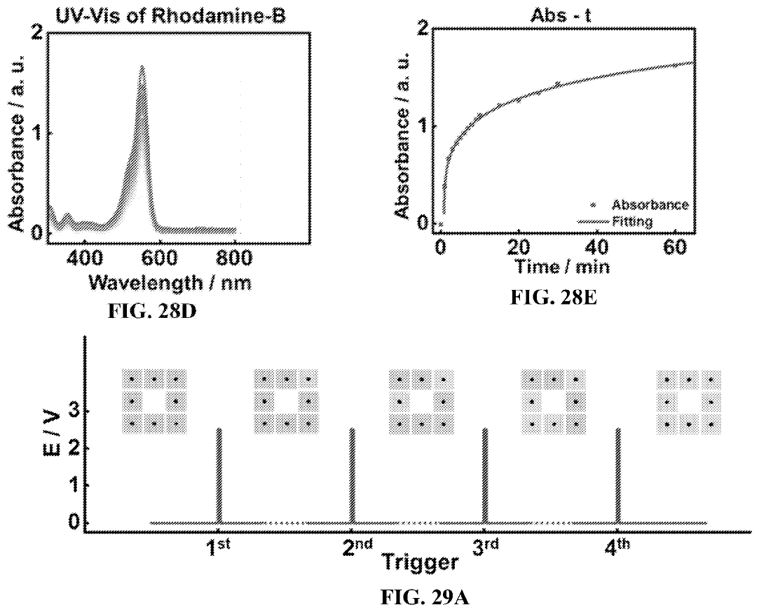

FIG. 28A to FIG. 28E depict an example characterization of dye release from microneedles (for example, 0.3% Rhodamine B loaded with 1.2 mm in length). FIG. 28A depicts example optical images of a microneedle array undergoing dye release from 0 to 60 minutes in 45° C. 1×DPBS. FIG. 28B depicts an example UV-Vis spectroscopy (for example, between 300 nm to 800 nm) of Rhodamine B standard solutions (including six samples). FIG. 28C depicts an example calibration curve of Rhodamine B from standard solutions in FIG. 28B. The parameters of the example calibration curve is summarized in the following Table 3:

TABLE 3
y = (−0.0299813) + (0.0951797) * x

| | Abs-conc. @, 556 nm |
|---|---|
| Pearson's r | 0.99989 |
| Adj. R-Square | 0.99972 |
| Intercept | −0.02998 plus/minus 0.00373 |
| Slope | 0.09518 plus/minus 7.16908E− |

FIG. 28D depicts an example UV-Vis spectroscopy of the environment solution corresponding to FIG. 28A (from 1 minute to 60 minutes as described above, where a darker line indicates a later time). FIG. 28E depicts an example absorbance versus time of environment solution from the dye release experiment.

FIG. 29A to FIG. 29C depict an example characterization of stepwise release control on single microneedles. FIG. 29A depicts an example schematic illustration of the electrical triggers at the single-needle level. In one or more embodiments, the microneedles (for example, with a length of 1.2 mm and having a gold coating with a thickness of 150 nm as an electrically triggerable membrane) on the array are separated from each other and triggered respectively. FIG. 29B depicts an example 8-needle device demonstration and optical images of the multistage triggering on single microneedles of the patch. In one or more embodiments, the microneedles triggered at the previous stage are labeled by red frames. FIG. 29C depicts example optical images of a 7-needle array before and after electrical triggering.

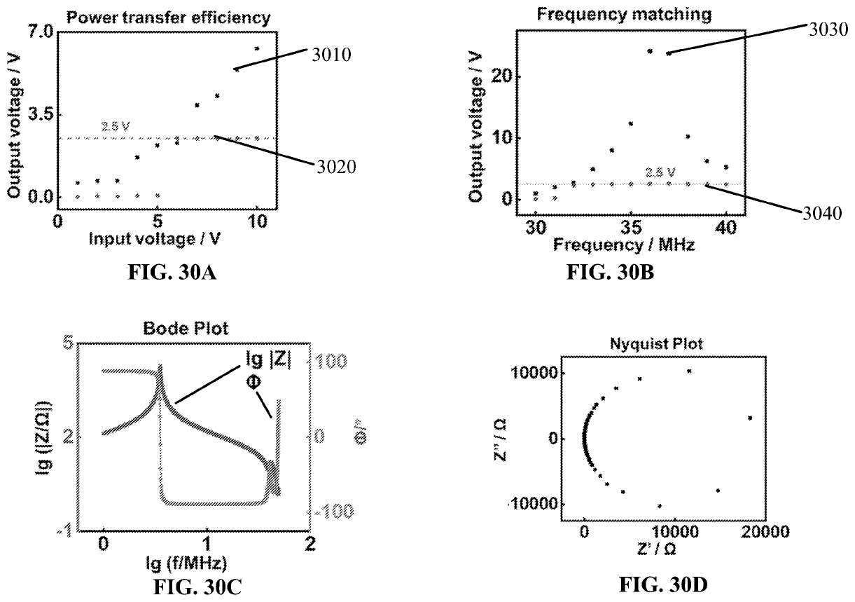

FIG. 30A to FIG. 30D depict an example characterization of the wireless power transfer module for SOP. FIG. 30A depicts example power transfer efficiency at 40 MHz. In one or more embodiments, the input signal refers to the signal applied on the inductive coil while the output signal refers to the signal after full-bridge rectification (shown by the dots 3010) and 2.5-V regulation (shown by the dots 3020). In one or more embodiments, a minimum input peak-to-peak voltage of 7 V is required to generate stable 2.5 V DC potential. FIG. 30B depicts example output voltage versus frequency with a 10-V (peak-to-peak) input signal, indicating an optimal transmitting frequency at around 36 MHz. In FIG. 30B, rectified output signal 3030 and 2.5-V regulated output signal 3040 are depicted. FIG. 30C to FIG. 30D depicts an example bode plot and Nyquist plot of the SOP system.

FIG. 31 depicts example representative confocal images of 40-μm horizontal cortical slices at various stages after implantation of the bioresorbable electrode probes. In one or more embodiments, probes were collected on days 1, 7 and 14, covering the typical lifetime of a bioresorbable device. In one or more embodiments, the images show cross sectional views of the implantation site with immunohistochemical staining for: a. Nissl bodies (neurotrace 3110); b. astrocytes (glial fibrillary acidic protein (GFAP) 3120); c. activated microglia (Ibal 3130) and d. DNA (4′,6-diamidino-2-phenylindole 3140 (also referred to as “DAPI”), and overall lesions from bioresorbable optical probes (n=3 independent experiments).

FIG. 32 depicts an example SOP coupled with a custom head stage that can be firmly mounted onto mice heads.

FIG. 33A to FIG. 33L depict example in vitro stimulation of brain with microneedles (for example, having a length of 3 mm and having a gold coating of 150 nm in thickness as an electrically triggerable membrane). For example, FIG. 33A and FIG. 33B depict an example signal (for example, 50 mV, 1 Hz, 10 ms in duration) recorded (“Rec.”) 5 mm (as shown in FIG. 33A) and 2 mm (as shown in FIG. 33B) away from the stimulation microneedle (“Stim.”) FIG. 33C to FIG. 33G depict an example 1 Hz stimulation (10 ms in duration) from 10 mV −50 mV recorded by two electrodes (for example, “Rec. 1” and “Rec. 2,” which are approximately 5 mm away from the microneedle). FIG. 33H to FIG. 33L depict the 10 Hz stimulation (for example, 1 ms in duration) from 10-50 mV recorded by two electrodes (for example, “Rec. 1” and “Rec. 2,” which are approximately 5 mm away from the microneedle).

FIG. 34A to FIG. 34G depict an example finite element analysis of a two-microneedle model. For example, FIG. 34A depicts the two-microneedle model (1.2-mm, 3.5-mm in distance, in a 5-mm cubic space) and current distribution (a.u.) at 2.5 V. In one or more embodiments, both current distributions in the electrolyte and on the surface are visualized. FIG. 34B to FIG. 34D depict an example iso-potential surface from different perspectives (for example, as shown in FIG. 34B and FIG. 34C), as well as potential distribution on the diagonal plane (for example, as shown in FIG. 34D) at 2.5 V. FIG. 34E depicts the anodic polarization curve with standardized current of the crevice corrosion from 2.0 V to 3.5 V. FIG. 34F to FIG. 34G depict an example corrosion rate of gold at 2.5 V and the corresponding visualization.

While the drawings and description above provide various example implementations in accordance various embodiments of the present disclosure, it is noted that various example implementations may be combined with one another. For example, various embodiments of the present disclosure provide a sensing apparatus for deep tissue sensing and transdermal delivery that comprises not only a base layer configured to interface with a skin surface of a subject and a sensing layer positioned above the base layer, but also a microneedle attached to a skin-interfacing portion of the base layer and an electrically triggerable membrane encapsulating the microneedle. In such examples, the sensing layer may comprise one or more waveform detectors and one or more waveform generators configured to emit wave signals, similar to various example implementations described above. The microneedle may be configured to waveguide the wave signals into a deep tissue of the subject, similar to the various example implementations described above. The electrically triggerable membrane may encapsulate the microneedle and define at least one reservoir between the microneedle and the electrically triggerable membrane, similar to various example implementations described above. In some embodiments, the sensing apparatus further comprises a controller coupled to the microneedle and configured to transmit an electrical trigger to the microneedle to cause a disintegration of the electrically triggerable membrane and a release of content from the at least one reservoir, similar to various example implementations described above. In some embodiments, the controller is configured to: receive a release control signal, and in response to the release control signal, transmit the electrical trigger to the microneedle, similar to various example implementations described above.

In some embodiments, the release control signal comprises a microneedle indication associated with the microneedle, similar to various examples described above. In some embodiments, the sensing apparatus further comprises a microneedle array comprising a plurality of microneedles that includes the microneedle, wherein the electrically triggerable membrane encapsulates each of the plurality of microneedles, similar to various example implementations described above. In some embodiments, the one or more waveform generators comprise one or more light-emitting diodes configured to emit light signals, similar to various example implementations described above. In some embodiments, the microneedle is configured as an optical waveguide for the light signals, similar to various example implementations described above. In some embodiments, the light signals include visible red light signals and near-infrared signals, similar to various example implementations described above. In some embodiments, the one or more waveform generators comprise one or more ultrasonic generators configured to emit ultrasonic signals, and wherein the microneedle is configured to act as ultrasonic waveguides for the ultrasonic signals, similar to various example implementations described above. In some embodiments, the sensing apparatus further comprises a controller in electronic communication with the one or more waveform generators and the one or more waveform detectors, and the controller is configured to: operate the one or more waveform generators to define a sensing field within the deep tissue of the subject via the wave signals and the microneedle as a waveguide; and generate sensing data based at least in part on reflected wave signals detected at the one or more waveform detectors, similar to various example implementations described above. In some embodiments, the controller is further configured to transmit, via wireless communication, the sensing data to a workstation, similar to various example implementations described above.

Many modifications and other embodiments of the present disclosure set forth herein will come to mind to one skilled in the art to which the present disclosures pertain having the benefit of the teachings presented in the foregoing descriptions and the associated drawings. Therefore, it is to be understood that the present disclosure is not to be limited to the specific embodiments disclosed and that modifications and other embodiments are intended to be included within the scope of the appended claim concepts. Although specific terms are employed herein, they are used in a generic and descriptive sense only and not for purposes of limitation.

1.-40. (canceled)

41. A sensing apparatus for deep tissue sensing comprising:
a base layer configured to interface with a skin surface of a subject;
a sensing layer positioned above the base layer and comprising one or more waveform detectors and one or more waveform generators configured to emit wave signals; and
a plurality of microneedles attached to a skin-interfacing portion of the base layer and oriented to extend into at least a dermal depth or a subcutaneous depth of the subject, wherein the plurality of microneedles is configured to waveguide the wave signals into a deep tissue of the subject.

42. The sensing apparatus of claim 41, wherein the one or more waveform generators comprise one or more light-emitting diodes configured to emit light signals, wherein the plurality of microneedles is configured as optical waveguides for the light signals, wherein the light signals include visible red light signals and near-infrared signals.

43. The sensing apparatus of claim 41, wherein the one or more waveform generators comprise one or more ultrasonic generators configured to emit ultrasonic signals, wherein the plurality of microneedles is configured to act as ultrasonic waveguides for the ultrasonic signals.

44. The sensing apparatus of claim 41, wherein the base layer and the plurality of microneedles are configured to minimize a transfer of ambient heat originating from the one or more waveform generators to the skin surface of the subject, wherein at least the base layer and the sensing layer form a flexible substrate configured to conform to contours of the skin surface of the subject, wherein the plurality of microneedles is comprised of biocompatible material with waveguiding properties.

45. The sensing apparatus of claim 41, further comprising:
a control module in electronic communication with the one or more waveform generators and the one or more waveform detectors, wherein the control module is positioned above the sensing layer and configured to:
operate the one or more waveform generators to define a sensing field within the deep tissue of the subject via the wave signals and the plurality of microneedles as waveguides;
generate sensing data based at least in part on reflected wave signals detected at the one or more waveform detectors;
process the sensing data to determine physiological measurements associated with the deep tissue of the subject; and
transmit, via wireless communication, the sensing data and/or the physiological measurements to a workstation.

46. The sensing apparatus of claim 45, wherein the physiological measurements are selected from the group consisting of at least one of tissue oximetry measurements, pulse oximetry measures, heart pulsation measurements, respiratory measurements, volume measurements, or plethysmographic measurements.

47. The sensing apparatus of claim 46, wherein the physiological measurements are determined from the sensing data using one or more machine learning models trained at least to reduce noise in the sensing data.

48. The sensing apparatus of claim 41, further comprising:
a control unit configured to generate and transmit, via wireless communication, sensing data based at least in part on reflected wave signals detected at the one or more waveform detectors;
a workstation configured to:
receive, via wireless communication, the sensing data from the sensing apparatus, and
determine a plurality of physiological measurements associated with the deep tissue of the subject from the sensing data.

49. An apparatus for transdermal delivery comprising:
a microneedle; and
an electrically triggerable membrane encapsulating the microneedle and defining at least one reservoir between the microneedle and the electrically triggerable membrane.

50. The apparatus of claim 49, wherein the electrically triggerable membrane comprises electrically triggerable gold, wherein a membrane width associated with the electrically triggerable membrane is between 145 nanometers and 155 nanometers.

51. The apparatus of claim 49, further comprising a controller coupled to the microneedle and configured to transmit an electrical trigger to the microneedle to cause a disintegration of the electrically triggerable membrane and a release of content from the at least one reservoir, wherein the electrical trigger comprises a direct current signal between 2 volts and 3 volts.

52. The apparatus of claim 51, wherein the controller is configured to:
receive a release control signal, wherein the release control signal comprises a microneedle indication associated with the microneedle, and
in response to the release control signal, transmit the electrical trigger to the microneedle, wherein the controller comprises at least one of a near-field communication module or a Bluetooth module.

53. The apparatus of claim 51 further comprising, a microneedle array comprising a plurality of microneedles that includes the microneedle, wherein the electrically triggerable membrane encapsulates each of the plurality of microneedles.

54. The apparatus of claim 53, wherein the controller is configured to:
receive a plurality of release control signals;
determine one or more microneedles from the plurality of microneedles that are associated with the plurality of release control signals; and
transmit one or more electrical triggers to the one or more microneedles.

55. A sensing apparatus for deep tissue sensing and transdermal delivery comprising:
a base layer configured to interface with a skin surface of a subject;
a sensing layer positioned above the base layer and comprising one or more waveform detectors and one or more waveform generators configured to emit wave signals;
a microneedle attached to a skin-interfacing portion of the base layer and configured to waveguide the wave signals into a deep tissue of the subject; and
an electrically triggerable membrane encapsulating the microneedle and defining at least one reservoir between the microneedle and the electrically triggerable membrane.

56. The sensing apparatus of claim 55, further comprising:
a controller coupled to the microneedle and configured to transmit an electrical trigger to the microneedle to cause a disintegration of the electrically triggerable membrane and a release of content from the at least one reservoir, wherein the controller is configured to:
receive a release control signal, wherein the release control signal comprises a microneedle indication associated with the microneedle, and
in response to the release control signal, transmit the electrical trigger to the microneedle.

57. The sensing apparatus of claim 55, further comprising a microneedle array comprising a plurality of microneedles that includes the microneedle, wherein the electrically triggerable membrane encapsulates each of the plurality of microneedles.

58. The sensing apparatus of claim 55, wherein the one or more waveform generators comprise one or more light-emitting diodes configured to emit light signals, wherein the microneedle is configured as an optical waveguide for the light signals, wherein the light signals include visible red light signals and near-infrared signals.

59. The sensing apparatus of claim 55, wherein the one or more waveform generators comprise one or more ultrasonic generators configured to emit ultrasonic signals, and wherein the microneedle is configured to act as ultrasonic waveguides for the ultrasonic signals.

60. The sensing apparatus of claim 55, further comprising:
a controller in electronic communication with the one or more waveform generators and the one or more waveform detectors, the controller is configured to:
operate the one or more waveform generators to define a sensing field within the deep tissue of the subject via the wave signals and the microneedle as a waveguide;
generate sensing data based at least in part on reflected wave signals detected at the one or more waveform detectors; and
transmit, via wireless communication, the sensing data to a workstation.
